(19) United States
(12) Patent Application Publication
(10) Pub. No.: US 2021/0285949 A1
(43) Pub. Date: Sep. 16, 2021

(54) PRO-ADRENOMEDULLIN OR FRAGMENT THEREOF IN PATIENTS INFECTED WITH CORONA VIRUS AND TREATMENTS WITH BINDER AGAINST ADRENOMEDULLIN

(71) Applicants: SphingoTec GmbH, Hennigsdorf (DE); AdrenoMed AG, Hennigsdorf (DE)

(72) Inventor: Andreas BERGMANN, Berlin (DE)

(73) Assignees: SphingoTec GmbH, Hennigsdorf (DE); AdrenoMed AG, Hennigsdorf (DE)

(21) Appl. No.: 17/201,518

(22) Filed: Mar. 15, 2021

Related U.S. Application Data
(60) Provisional application No. 63/142,370, filed on Jan. 27, 2021.
(60) Provisional application No. 63/015,102, filed on Apr. 24, 2020.
(60) Provisional application No. 62/990,171, filed on Mar. 16, 2020.

(30) Foreign Application Priority Data
Mar. 16, 2020 (EP) 20163406.0
Jun. 12, 2020 (EP) 20179738.8
Jan. 27, 2021 (EP) 21153847.5

(51) Int. Cl. G01N 33/569; G01N 33/68; C07K 16/22; A61P 31/14

(52) U.S. Cl. CPC G01N 33/56983; G01N 33/6893; C07K 16/22; A61P 31/14; A61K 2039/505; G01N 2800/50; G01N 2800/56; G01N 2800/52; C07K 2317/565; G01N 2333/165

(57) ABSTRACT

Subject matter of the present invention is a method for (a) diagnosing or predicting the risk of life-threatening deterioration or an adverse event or (b) prognosing the severity or (c) predicting or monitoring the success of a therapy or intervention in a patient infected with a Corona virus, the method comprising:
determining the level of pro-Adrenomedullin (SEQ ID No. 31) or fragment thereof in a sample of bodily fluid of said patient,
comparing said level of pro-Adrenomedullin or fragment thereof to a pre-determined threshold or a previous level of pro-Adrenomedullin or fragment thereof, and
correlating said level of pro-Adrenomedullin or fragment thereof with the risk of life-threatening deterioration or an adverse event, or
correlating said level of pro-Adrenomedullin or fragment thereof with the severity, or
correlating said level of pro-Adrenomedullin or fragment thereof with the success of a therapy or intervention,
   wherein said pro-Adrenomedullin or fragment thereof is selected from the group consisting of PAMP (SEQ ID No. 32), MR-proADM (SEQ ID No. 33), ADM-NH2 (SEQ ID No. 20), ADM-Gly (SEQ ID No. 21) and CT-proADM (SEQ ID No. 34).

Subject matter of the present invention is an Anti-adrenomedullin (ADM) antibody or anti-ADM antibody fragment or anti-ADM non-Ig scaffold for use in therapy or intervention in a patient in a patient infected with a Corona virus.

### FIELD OF THE INVENTION

Subject matter of the present invention is a method for (a) diagnosing or predicting the risk of life-threatening deterioration or an adverse event or (b) diagnosing or prognosing the severity or (c) predicting or monitoring the success of a therapy or intervention or (d) therapy guidance or therapy stratification or (e) patient management in a patient infected with a Corona virus, the method comprising:
determining the level of pro-Adrenomedullin (SEQ ID No. 31) or fragment thereof in a sample of bodily fluid of said patient,
comparing said level of pro-Adrenomedullin or fragment thereof to a pre-determined threshold or to a previously measured level of pro-Adrenomedullin or fragment thereof, and
correlating said level of pro-Adrenomedullin or fragment thereof with the risk of life-threatening deterioration or an adverse event, or
correlating said level of pro-Adrenomedullin or fragment thereof with the severity, or
correlating said level of pro-Adrenomedullin or fragment thereof with the success of a therapy or intervention, or
correlating said level of pro-Adrenomedullin or fragment thereof with a certain therapy or intervention, or
correlating said level of pro-Adrenomedullin or fragment thereof with the management of said patient,

wherein said pro-Adrenomedullin or fragment thereof is selected from the group consisting of PAMP (SEQ ID No. 32), MR-proADM (SEQ ID No. 33), ADM-NH₂ (SEQ ID No. 20), ADM-Gly (SEQ ID No. 21) and CT-proADM (SEQ ID No. 34).

Subject matter of the present invention is an Anti-adrenomedullin (ADM) antibody or anti-ADM antibody fragment or anti-ADM non-Ig scaffold for use in therapy or intervention in a patient in a patient infected with a Corona virus.

### BACKGROUND

The peptide adrenomedullin (ADM) was described for the first time in 1993 (Kitamura et al., 1993. Biochem Biophys Res Comm 192 (2): 553-560) as a novel hypotensive peptide comprising 52 amino acids, which had been isolated from a human pheochromocytoma cell line (SEQ ID No. 20). In the same year, cDNA coding for a precursor peptide comprising 185 amino acids and the complete amino acid sequence of this precursor peptide were also described. The precursor peptide, which comprises, inter alia, a signal sequence of 21 amino acids at the N-terminus, is referred to as “pre-proadrenomedullin” (pre-proADM). In the present description, all amino acid positions specified usually relate to the pre-proADM, which comprises the 185 amino acids. Pre-proADM is subsequently converted into the 164 amino acid pro-ADM (SEQ ID No. 31) by cleavage of the N-terminal signal-peptide. Pro-ADM is further processed into pro-ADM N-terminal 20 peptide (PAMP; SEQ ID No. 32), midregional pro-ADM (MR-proADM; SEQ ID No. 33), adrenotensin pro-ADM 153-185 (CT-pro ADM; SEQ ID No. 34) and immature ADM, a C-terminally glycine-extended version of ADM (ADM-Gly; SEQ ID No. 21). This is converted into the mature bioactive form of ADM (bio-ADM; ADM-NH₂; SEQ ID No. 20) by enzymatic amidation of its C-terminus. More than half of the known neural and endocrine peptides require the formation of a C-terminal alpha-amide group to gain full biological activity (Guembe et al. 1999. J Histochem Cytochem 47(5): 623-36; Vishwanatha et al. 2013. Handbook of Biologically Active Peptides Peptidylglycine Amidating Monoxygenase (PAM). Second Edi. Elsevier Inc.). This final step of peptide hormone biosynthesis involves the action of a bifunctional enzyme, the peptidylglycine alpha-amidating monooxygenase (PAM), that specifically recognizes C-terminal glycine (CT-Gly) residues in its substrates. PAM cleaves glyoxylate from the peptides CT-Gly residue in a two-step enzymatic reaction leading to the formation of c-terminally alpha-amidated peptide hormones, wherein the resulting alpha-amide group originates from the cleaved CT-Gly (Prigge et al. 2000. Cellular and Molecular Life Sciences 57(8): 1236-59). This amidation reaction takes place in the lumen of secretory granules prior to exocytosis of the amidated product (Martinez et al. 1996. Am J Pathol 149(2):707-16).

The discovery and characterization of ADM in 1993 triggered intensive research activity, the results of which have been summarized in various review articles, in the context of the present description, reference being made in particular to the articles to be found in an issue of “Peptides” devoted to ADM in particular (Takahashi 2001. Peptides 22: 1691; Eto 2001. Peptides 22: 1693-1711). A further review is Hinson et al. 2000 (Hinson et al. 2000. Endocrine Reviews 21(2):138-167). In the scientific investigations to date, it has been found, inter alia, that ADM may be regarded as a polyfunctional regulatory peptide. As mentioned above, it is released into the circulation in an inactive form extended by glycine (Kitamura et al. 1998. Biochem Biophys Res Commun 244(2): 551-555). There is also a binding protein (Pio et al. 2001. The Journal of Biological Chemistry 276(15): 12292-12300), which is specific for ADM and probably likewise modulates the effect of ADM. Those physiological effects of ADM as well as of PAMP, which are of primary importance in the investigations to date, were the effects influencing blood pressure.

Hence, ADM is an effective vasodilator, and thus it is possible to associate the hypotensive effect with the particular peptide segments in the C-terminal part of ADM. It has furthermore been found that the above-mentioned physiologically active peptide PAMP formed from pre-proADM likewise exhibits a hypotensive effect, even if it appears to have an action mechanism differing from that of ADM (in addition to the mentioned review articles above, Eto et al. 2001 and Hinson et al. 2000 see also Kuwasaki et al. 1997. FEBS Lett 414(1): 105-110; Kuwasaki et al. 1999. Ann. Clin. Biochem. 36: 622-628; Tsuruda et al. 2001 Life Sci. 69(2): 239-245 and EP A2 0 622 458). It has furthermore been found, that the concentrations of ADM, which can be measured in the circulation and other biological liquids, are in a number of pathological states, significantly above the concentrations found in healthy control subjects. Thus, the ADM level in patients with congestive heart failure, myocardial infarction, kidney diseases, hypertensive disorders, diabetes mellitus, in the acute phase of shock and in sepsis and septic shock are significantly increased, although to different extents. The PAMP concentrations are also increased in some of said pathological states, but the plasma levels are lower relative to ADM (Eto 2001. Peptides 22: 1693-1711). It was reported that high concentrations of ADM are observed in sepsis, and the highest concentrations in septic shock (Eto 2001. Peptides 22: 1693-1711; Hirata et al. Journal of Clinical Endocrinology and Metabolism 81(4): 1449-1453; Ehlenz et al. 1997. Exp Clin Endocrinol Diabetes 105: 156-162; Tomoda et al. 2001. Peptides 22: 1783-1794; Ueda et al. 1999. Am. J. Respir. Crit. Care Med. 160: 132-136 and Wang et al. 2001. Peptides 22: 1835-1840). Moreover, plasma concentrations of ADM are elevated in patients with heart failure and correlate with disease severity (Hirayama et al. 1999. J Endocrinol 160: 297-303; Yu et al. 2001. Heart 86: 155-160). High plasma ADM is an independent negative prognostic indicator in these subjects (Poyner et al. 2002. Pharmacol Rev 54: 233-246).

Kitamura and colleagues showed that the concentration of mature ADM and ADM-Gly was significantly elevated in plasma of hypertensive patients compared to healthy volunteers (Kitamura et al. 1998. Biochem Biophys Res Comm 244(2): 551-5). In both groups mature ADM was much lower than ADM-Gly. However, the ratio of mature ADM to ADM-Gly was not significantly different between hypertensive and non-hypertensive subjects.

It is reported for the early phase of sepsis, that ADM improves heart function and the blood supply in liver, spleen, kidney and small intestine. Anti-ADM-neutralizing antibodies neutralize the before mentioned effects during the early phase of sepsis (Wang et al. 2001. Peptides 22: 1835-1840). For other diseases, blocking of ADM may be beneficial to a certain extent. However, it might also be detrimental if ADM is totally neutralized, as a certain amount of ADM may be required for several physiological functions. In many reports it was emphasized, that the administration of ADM may be beneficial in certain diseases. In contrast thereto, in other reports ADM was reported as being life threatening when administered in certain conditions.

WO2013/072510 describes a non-neutralizing N-terminal anti-ADM antibody for use in therapy of a severe chronical or acute disease or acute condition of a patient for the reduction of the mortality risk for said patient.

WO2013/072511 describes a non-neutralizing N-terminal anti-ADM antibody for use in therapy of a chronical or acute disease or acute condition of a patient for prevention or reduction of organ dysfunction or organ failure.

WO2013/072513 describes a N-terminal anti-ADM antibody for use in therapy of an acute disease or condition of a patient for stabilizing the circulation.

WO2013/072514 describes a N-terminal anti-ADM antibody for regulating the fluid balance in a patient having a chronic or acute disease or acute condition.

WO2019/154900 describes a non-neutralizing N-terminal anti-ADM antibody for use in therapy and prevention of dementia. Moreover, WO2019/154900 describes a method for diagnosing and monitoring a (preventive) therapy of dementia by determining a ratio of the level of mature ADM to the level of pro-Adrenomedullin or a fragment thereof.

WO2013/072512 describes a non-neutralizing N-terminal anti-ADM antibody that is an ADM stabilizing antibody enhancing the half-life (t_{1/2} half retention time) of adrenomedullin in serum, blood, plasma.

The efficacy of non-neutralizing antibody targeted against the N-terminus of ADM was investigated in a survival study in CLP-induced sepsis in mice. Pre-treatment with the non-neutralizing antibody resulted in decreased catecholamine infusion rates, kidney dysfunction, and ultimately improved survival (Struck et al. 2013. Intensive Care Med Exp 1(1):22; Wagner et al. 2013. Intensive Care Med Exp 1(1):21). In addition, antibodies against the mid-regional part of ADM (MR-ADM antibodies) also significantly improved the survival in mice with CLP-induced sepsis, but to a lower extent when compared to N-terminal anti-ADM antibodies (Struck et al. 2013. Intensive Care Med Exp 1(1):22).

Due to these positive results, a humanized version of an N-terminal anti-ADM antibody, named Adrecizumab, has been developed for further clinical development. Beneficial effects of Adrecizumab on vascular barrier function and survival were recently demonstrated in preclinical models of systemic inflammation and sepsis (Geven et al. 2018. Shock 50(6):648-654). In this study, pre-treatment with Adrecizumab attenuated renal vascular leakage in endotoxemic rats as well as in mice with CLP-induced sepsis, which coincided with increased renal expression of the protective peptide Ang-1 and reduced expression of the detrimental peptide vascular endothelial growth factor. Also, pre-treatment with Adrecizumab improved 7-day survival in CLP-induced sepsis in mice from 10 to 50% for single and from 0 to 40% for repeated dose administration. Moreover, in a phase I study, excellent safety and tolerability was demonstrated: no serious adverse events were observed, no signal of adverse events occurring more frequently in Adrecizumab-treated subjects was detected and no relevant changes in other safety parameters were found (Geven et al. 2017. Intensive Care Med Exp 5 (Suppl 2): 0427). Of particular interest is the proposed mechanism of action of Adrecizumab. Both animal and human data reveal a potent, dose-dependent increase of circulating ADM following administration of this antibody. Based on pharmacokinetic data and the lack of an increase in MR-proADM (an inactive peptide fragment derived from the same prohormone as ADM), the higher circulating ADM levels cannot be explained by an increased production.

A mechanistic explanation for this increase could be that the excess of antibody in the circulation may drain ADM from the interstitium to the circulation, since ADM is small enough to cross the endothelial barrier, whereas the antibody is not (Geven et al. 2018. Shock. 50(2): 132-140). In addition, binding of the antibody to ADM leads to a prolongation of ADM's half-life. Even though NT-ADM antibodies partially inhibit ADM-mediated signalling, a large increase of circulating ADM results in an overall “net” increase of ADM activity in the blood compartment, where it exerts beneficial effects on ECs (predominantly barrier stabilization), whereas ADMs detrimental effects on VSMCs (vasodilation) in the interstitium are reduced.

In other words, by increasing functional plasma ADM levels, NT-ADM antibodies are hypothesized to target the sepsis- and inflammation-based vascular and capillary leakage. The latter leads to deterioration of severe COVID-19 to septic shock and ARDS (Veerdonk et al. 2020. Preprints, 2020040023 (doi: 10.20944/preprints202004.0023.v1)). Very recently, stabilization of the endothelium has been explicitly identified as a therapeutic goal in COVID-19 (Varga et al. 2020.395(10234): 1417-1418).

An N-terminal ADM antibody, named Adrecizumab (HAM 8101) was administered to eight extreme-critically ill COVID-19 patients with acute respiratory distress syndrome (ARDS) (Karakas et al. 2020. Biomolecules 10: 1171). The patients received a single dose of Adrecizumab, which was administered between 1 and 3 days after the initiation of mechanical ventilation. The SOFA (median 12.5) and SAPS-II (median 39) scores clearly documented the population at highest risk. Follow-up ranged between 13 and 27 days. Following the Adrecizumab administration, one patient in the low-dose group died at day 4 due to fulminant pulmonary embolism, while four were in stable condition, and three were discharged from the intensive care unit (ICU). Within 12 days, the SOFAscore, as well as the disease severity score (range 0-16, mirroring critical resources in the ICU, with higher scores indicating more severe illness), decreased in five out of the seven surviving patients (in all high-dose patients). The PaO2/FiO2 increased within 12 days, while the inflammatory parameters C-reactive protein, procalcitonin, and interleukin-6 decreased. Importantly, the mortality was lower than expected and calculated by the SOFA score. In conclusion, in this preliminary uncontrolled case series of eight shock patients with life-threatening COVID-19 and ARDS, the administration of Adrecizumab was followed by a favorable outcome.

Corona viruses are widespread in humans and several other vertebrates and cause respiratory, enteric, hepatic, and neuro logic diseases. Notably, the severe acute respiratory syndrome coronavirus (SARS-CoV) in 2003 and Middle East respiratory syndrome coronavirus (MERS-CoV) in 2012 have caused human epidemics. Comparison with the SARS-CoV shows several significant differences and similarities. Both MERS CoV and SARS-CoV have much higher case fatality rates (40% and 10%, respectively) (de Wit et al. 2016. SARS and MERS: recent insights into emerging coronaviruses. Nat Rev Microbiol 14(8):523-34; Zhou et al. 2020. A pneumonia outbreak associated with a new coronavirus of probable bat origin. Nature 579(7798):270-273). Though the current SARS CoV-2 shares 79% of its genome with SARS-CoV, it appears to be much more transmissible. Both SARS-CoVs enter the cell via the angiotensin converting enzyme 2 (ACE2) receptor (Wan et al. 2020. Receptor recognition by novel coronavirus from Wuhan: An analysis based on decade-long structural studies of SARS. J Virol 94(7):e00127-20). The disease caused by SARS-CoV-2 is called corona-virus-disease 2019 (COVID-19).

The SARS-CoV-2 first predominantly infects lower airways and binds to ACE2 on alveolar epithelial cells. Both viruses are potent inducers of inflammatory cytokines. The “cytokine storm” or “cytokine cascade” is the postulated mechanism for organ damage. The virus activates immune cells and induces the secretion of inflammatory cytokines and chemokines into pulmonary vascular endothelial cells.

The clinical spectrum of SARS-CoV-2 infection appears to be wide, encompassing asymptomatic infection, mild upper respiratory tract illness, and severe viral pneumonia with respiratory failure and even death, with many patients being hospitalised with pneumonia (Huang et al. 2020 Clinical features of patients infected with 2019 novel coronavirus in Wuhan, China. Lancet 395: 497-506; Wang et al. 2020 Clinical characteristics of 138 hospitalized patients with 2019 novel coronavirus-infected pneumonia in Wuhan, China. JAMA 323(11):1061-1069; Chen et al. 2020. Epidemiological and clinical characteristics of 99 cases of 2019 novel coronavirus pneumonia in Wuhan, China: a descriptive study. Lancet 395: 507-13).

Very recently, older age, elevated d-dimer levels, and high SOFA score were proposed to help clinicians to identify at an early stage those patients with COVID-19 who have poor prognosis (Zhou et al. 2020. Clinical course and risk factors for mortality of adult inpatients with COVID-19 in Wuhan, China: a retrospective cohort study. The Lancet, 395(10229): 1054-1062).

Subject matter of the present invention is a method for (a) diagnosing or predicting the risk of life-threatening deterioration or an adverse event or (b) diagnosing or prognosing the severity or (c) predicting or monitoring the success of a therapy or intervention or (d) therapy guidance or therapy stratification or (e) patient management in a patient infected with a Corona virus, the method comprising:
determining the level of pro-Adrenomedullin (SEQ ID No. 31) or fragment thereof in a sample of bodily fluid of said patient,
comparing said level of pro-Adrenomedullin or fragment thereof to a pre-determined threshold or to a previously measured level of pro-Adrenomedullin or fragment thereof, and
correlating said level of pro-Adrenomedullin or fragment thereof with the risk of life-threatening deterioration or an adverse event, or
correlating said level of pro-Adrenomedullin or fragment thereof with the severity, or
correlating said level of pro-Adrenomedullin or fragment thereof with the success of a therapy or intervention, or
correlating said level of pro-Adrenomedullin or fragment thereof with a certain therapy or intervention, or
correlating said level of pro-Adrenomedullin or fragment thereof with the management of said patient,

wherein said pro-Adrenomedullin or fragment thereof is selected from the group consisting of PAMP (SEQ ID No. 32), MR-proADM (SEQ ID No. 33), ADM-NH₂ (SEQ ID No. 20), ADM-Gly (SEQ ID No. 21) and CT-proADM (SEQ ID No. 34).

Subject matter of the present invention is a method for (a) diagnosing or predicting the risk of life-threatening deterioration or an adverse event or (b) diagnosing or prognosing the severity or (c) predicting or monitoring the success of a therapy or intervention or (d) therapy guidance or therapy stratification or (e) patient management in a patient infected with a Corona virus, wherein said Corona Virus is selected from the group comprising Sars-CoV-1, Sars-CoV-2, MERS-CoV, in particular Sars-CoV-2.

Subject matter of the present is a method for (a) diagnosing or predicting the risk of life-threatening deterioration or an adverse event or (b) diagnosing or prognosing the severity or (c) predicting or monitoring the success of a therapy or intervention or (d) therapy guidance or therapy stratification or (e) patient management in a patient infected with a Corona virus according to the present invention, wherein said adverse event is selected from the group comprising death, organ dysfunction, shock, ARDS and ALI (Acute Lung Injury).

Subject matter of the present invention is a method for (a) diagnosing or predicting the risk of He-m threatening deterioration or an adverse event or (b) diagnosing or prognosing the severity or (c) predicting or monitoring the success of a therapy or intervention or (d) therapy guidance or therapy stratification or (e) patient management in a patient infected with a Corona virus according to the present invention, wherein said level of pro-Adrenomedullin or fragment thereof is above a pre-determined threshold.

In a specific embodiment of the present invention said level of pro-Adrenomedullin or fragment thereof is determined at least twice.

In another specific embodiment of the present invention said at least second determination of the level of pro-Adrenomedullin or fragment thereof is determined within 2 hours, preferably within 4 hours, more preferred within 6 hours, even more preferred within 12 hours, even more preferred within 24 hours, most preferred within 48 hours.

This means that according to the term “a previously measured level of pro-Adrenomedullin or fragment thereof” it is understood throughout all subject matters of the invention that said previously measured level is a level that has been measured within 2 hours, preferably within 4 hours, more preferred within 6 hours, even more preferred within 12 hours, even more preferred within 24 hours, most preferred within 48 hours. The difference between a measurement and a previously measurement is a relative difference between said level of pro-Adrenomedullin or fragment thereof in different samples taken from said patient at different time-points.

Bio-ADM≥70 pg/mL or ≥25% increase until the end of the next day (with a minimum of 50 pg/mL at all).

In another specific embodiment of the present invention said level of pro-Adrenomedullin or fragment thereof is determined in different samples taken from said patient at different time-points.

In another specific embodiment of the present invention the difference between said level of pro-Adrenomedullin or fragment thereof in different samples taken from said patient at different time-points is determined. The difference may be determined as absolute or relative difference.

In another specific embodiment of the present invention a therapy is initiated when said relative difference between said level of pro-Adrenomedullin or fragment thereof in different samples taken from said patient at different time-points is 100% or above, more preferred 75% or above, even more preferred 50% or above, most preferred 25% or above.

In another specific embodiment of the present invention a therapy is initiated when said relative level of pro-Adrenomedullin or fragments thereof is at least 25% and the absolute level of pro-Adrenomedullin or fragments thereof is at least 50 pg/ml in said second or further determination and said fragment of pro-Adrenomedullin is mature ADM (ADM-NH₂).

Subject matter of the present invention is a method for (a) diagnosing or predicting the risk of life-threatening deterioration or an adverse event or (b) diagnosing or prognosing the severity or (c) predicting or monitoring the success of a therapy or intervention or (d) therapy guidance or therapy stratification or (e) patient management in a patient infected with a Corona virus according to the present invention, wherein said fragment is MR-proADM (SEQ ID No. 33), and the predetermined threshold of MR-proADM in a sample of bodily fluid of said subject is between 0.5 and 2 nmol/L, preferably between 0.7 and 1.5 nmol/L, preferably between 0.8 and 1.2 nmol/L, most preferred a threshold of 1 nmol/L is applied.

Subject matter of the present invention is a method for (a) diagnosing or predicting the risk of life-threatening deterioration or an adverse event or (b) diagnosing or prognosing the severity or (c) predicting or monitoring the success of a therapy or intervention or (d) therapy guidance or therapy stratification or (e) patient management in a patient infected with a Corona virus according to the present invention, wherein said fragment is ADM-NH₂ (SEQ ID No. 20), and the predetermined threshold of ADM-NH₂ (SEQ ID No. 20) in a sample of bodily fluid of said subject is between 40 and 100 pg/mL, more preferred between 50 and 90 pg/mL, even more preferred between 60 and 80 pg/mL, most preferred said threshold is 70 pg/mL.

Subject matter of the present invention is a method for (a) diagnosing or predicting the risk of He-m threatening deterioration or an adverse event or (b) diagnosing or prognosing the severity or (c) predicting or monitoring the success of a therapy or intervention or (d) therapy guidance or therapy stratification or (e) patient management in a patient infected with a Corona virus according to the present invention, wherein said patient has a SOFA score equal or greater than 3, preferably equal or greater than 7 or a quick SOFA score equal or greater than 1, preferably equal or greater than 2.

Subject matter of the present invention is a method for (a) diagnosing or predicting the risk of life-threatening deterioration or an adverse event or (b) diagnosing or prognosing the severity or (c) predicting or monitoring the success of a therapy or intervention or (d) therapy guidance or therapy stratification or (e) patient management in a patient infected with a Corona virus according to the present invention, wherein said patient has a level of D-dimer equal or greater than 0.5 μg/ml, preferably equal or greater than 1 μg/ml.

Subject matter of the present invention is a method for (a) diagnosing or predicting the risk of life-threatening deterioration or an adverse event or (b) diagnosing or prognosing the severity or (c) predicting or monitoring the success of a therapy or intervention or (d) therapy guidance or therapy stratification or (e) patient management in a patient infected with a Corona virus according to the present invention, wherein the level of pro-Adrenomedullin or fragment thereof is determined by contacting said sample of bodily fluid with a capture binder that binds specifically to pro-Adrenomedullin or fragment thereof.

Subject matter of the present invention is a method for (a) diagnosing or predicting the risk of He-m threatening deterioration or an adverse event or (b) diagnosing or prognosing the severity or (c) predicting or monitoring the success of a therapy or intervention or (d) therapy guidance or therapy stratification or (e) patient management in a patient infected with a Corona virus according to the present invention, wherein said determination comprises the use of a capture-binder that binds specifically to pro-Adrenomedullin or fragment thereof wherein said capture-binder may be selected from the group of antibody, antibody fragment or non-IgG scaffold.

Subject matter of the present invention is a method for (a) diagnosing or predicting the risk of life-threatening deterioration or an adverse event or (b) diagnosing or prognosing the severity or (c) predicting or monitoring the success of a therapy or intervention or (d) therapy guidance or therapy stratification or (e) patient management in a patient infected with a Corona virus according to the present invention, wherein the level of pro-Adrenomedullin or fragment thereof is determined in a bodily fluid sample of said subject and wherein said determination comprises the use of a capture-binder that binds specifically to pro-Adrenomedullin or fragment thereof wherein said capture-binder is an antibody.

Subject matter of the present invention is a method for (a) diagnosing or predicting the risk of life-threatening deterioration or an adverse event or (b) diagnosing or prognosing the severity or (c) predicting or monitoring the success of a therapy or intervention or (d) therapy guidance or therapy stratification or (e) patient management in a patient infected with a Corona virus according to the present invention, wherein the level of pro-Adrenomedullin or fragment thereof is determined in a bodily fluid sample of said subject and wherein said determination comprises the use of a capture-binder that binds specifically to level of pro-Adrenomedullin or fragment thereof, wherein said capture-binder is immobilized on a surface.

Subject matter of the present invention is a method for (a) diagnosing or predicting the risk of fife-threatening deterioration or an adverse event or (b) diagnosing or prognosing the severity or (c) predicting or monitoring the success of a therapy or intervention or (d) therapy guidance or therapy stratification or (e) patient management in a patient infected with a Corona virus according to the present invention, wherein said patient is treated with an Anti-adrenomedullin (ADM) antibody or anti-ADM antibody fragment or anti-ADM non-Ig scaffold wherein said anti-ADM antibody or anti-ADM fragment or anti-ADM non-Ig scaffold binds to the N-terminal and/or mid-regional part (aa 1-42) of ADM-Gly and/or ADM-NH₂:

(SEQ ID No. 23)
YRQSMNNFQGLRSFGCRFGTCTVQKLAHQIYQFTDKDKDNVA,

Subject matter of the present invention is an Anti-adrenomedullin (ADM) antibody or anti-ADM antibody fragment or anti-ADM non-Ig scaffold for use in therapy or intervention in a patient infected with a Corona virus.

Subject matter of the present invention is an Anti-adrenomedullin (ADM) antibody or anti-ADM antibody fragment or anti-ADM non-Ig scaffold for use in therapy or intervention in a patient infected with a corona virus according to the present inventions, wherein said corona virus is selected from the group comprising Sars-CoV-1, Sars-CoV-2, MERS-CoV, in particular Sars-CoV-2.

Subject matter of the present invention is an Anti-adrenomedullin (ADM) antibody or anti-ADM antibody fragment or anti-ADM non-Ig scaffold for use in therapy or intervention in a patient infected with a Corona virus according to the present invention, wherein said patient has a level of pro-Adrenomedullin or fragment thereof in a sample of bodily fluid of said subject that is above a predetermined threshold or that is higher to a previously measured level of pro-Adrenomedullin or fragment thereof when determined by a method according to method as described above.

Subject matter of the present invention is an Anti-adrenomedullin (ADM) antibody or anti-ADM antibody fragment or anti-ADM non-Ig scaffold for use in therapy or intervention in a patient infected with a Corona virus according to the present invention, wherein said patient has a SOFA score equal or greater than 3, preferably equal or greater than 7 or a quick SOFA score equal or greater than 1, preferably equal or greater than 2.

Subject matter of the present invention is an Anti-adrenomedullin (ADM) antibody or anti-ADM antibody fragment or anti-ADM non-Ig scaffold for use in therapy or intervention in a patient infected with a Corona virus according to the present invention, wherein said patient has a level of D-dimer equal or greater than 0.5 μg/ml, preferably equal or greater than 1 μg/ml.

Subject matter of the present invention is an Anti-adrenomedullin (ADM) antibody or anti-ADM antibody fragment or anti-ADM non-Ig scaffold for use in therapy or intervention in a patient infected with a Corona virus according to the present invention, wherein said anti-ADM antibody or anti-ADM fragment or anti-ADM non-Ig scaffold binds to the N-terminal part (amino acid 1-21) of ADM-Gly and/or ADM-NH₂: YRQSMNNFQGLRSFGCRFGTC (SEQ ID No. 14).

Subject matter of the present invention is an Anti-adrenomedullin (ADM) antibody or anti-ADM antibody fragment or anti-ADM non-Ig scaffold for use in therapy or intervention in a patient infected with a Corona virus according to the present invention, wherein said Anti-adrenomedullin (ADM) antibody or anti-ADM antibody fragment or anti-ADM non-Ig scaffold exhibits a minimum binding affinity to pro-Adrenomedullin or a fragment thereof of equal or less than 10⁻⁷ M.

Subject matter of the present invention is an Anti-adrenomedullin (ADM) antibody or anti-ADM antibody fragment or anti-ADM non-Ig scaffold for use in therapy or intervention in a patient infected with a Corona virus according to the present invention, wherein said Anti-adrenomedullin (ADM) antibody or anti-ADM antibody fragment or anti-ADM non-Ig scaffold wherein said antibody or fragment or scaffold blocks the bioactivity of ADM not more than 80%, preferably not more than 50%.

Subject matter of the present invention is an Anti-adrenomedullin (ADM) antibody or anti-ADM antibody fragment or anti-ADM non-Ig scaffold for use in therapy or intervention in a patient infected with a Corona virus according to the present invention, wherein said antibody is a monoclonal antibody or monoclonal antibody fragment.

Subject matter of the present invention is an Anti-adrenomedullin (ADM) antibody or anti-ADM antibody fragment or anti-ADM non-Ig scaffold for use in therapy or intervention in a patient infected with a Corona virus according to the present invention, wherein the complementarity determining regions (CDR's) in the heavy chain comprises the sequences:

CDR1:
SEQ ID NO: 1
GYTFSRYW
CDR2:
SEQ ID NO: 2
ILPGSGST
CDR3:
SEQ ID NO: 3
TEGYEYDGFDY

and the complementarity determining regions (CDR's) in the light chain comprises the sequences:

CDR1:
SEQ ID NO: 4
QSIVYSNGNTY
CDR2:
RVS
CDR3:
SEQ ID NO: 5
FQGSHIPYT

Subject matter of the present invention is an Anti-adrenomedullin (ADM) antibody or anti-ADM antibody fragment or anti-ADM non-Ig scaffold for use in therapy or intervention in a patient infected with a Corona virus according to the present invention, wherein said antibody or fragment comprises a sequence selected from the group comprising as a VH region:

(AM-VH-C)
SEQ ID NO: 6
QVQLQQSGAELMKPGASVKISCKATGYTFSRYWIEWVKQRPGHGLEWIG
EILPGSGSTNYNEKFKGKATITADTSSNTAYMQLSSLTSEDSAVYYCTE
GYEYDGFDYWGQGTTLTVSSASTKGPSVFPLAPSSKSTSGGTAALGCLV
KDYFPEPVTVSWNSGALTSGVHTFPAVLQSSGLYSLSSVVTVPSSSLGT
QTYICNVNEIKPSNTKVDKRVEPK
(AM-VH1)
SEQ ID NO: 7
QVQLVQSGAEVKKPGSSVKVSCKASGYTFSRYWISWVRQAPGQGLEWMG
RILPGSGSTNYAQKFQGRVTITADESTSTAYMELSSLRSEDTAVYYCTE
GYEYDGFDYWGQGTTVTVSSASTKGPSVFPLAPSSKSTSGGTAALGCLV
KDYFPEPVTVSWNSGALTSGVHTFPAVLQSSGLYSLSSVVTVPSSSLGT
QTYICNVNEEKPSNTKVDKRVEPK
(AM-VH2-E40)
SEQ ID NO: 8
QVQLVQSGAEVKKPGSSVKVSCKASGYTFSRYWIEWVRQAPGQGLEWMG
RILPGSGSTNYAQKFQGRVTITADESTSTAYMELSSLRSEDTAVYYCTE
GYEYDGFDYWGQGTTVTVSSASTKGPSVFPLAPSSKSTSGGTAALGCLV
KDYFPEPVTVSWNSGALTSGVHTFPAVLQSSGLYSLSSVVTVPSSSLGT
QTYICNVNEEKPSNTKVDKRVEPK
(AM-VH3-T26-E55)
SEQ ID NO: 9
QVQLVQSGAEVKKPGSSVKVSCKATGYTFSRYWISWVRQAPGQGLEWMG
EILPGSGSTNYAQKFQGRVTITADESTSTAYMELSSLRSEDTAVYYCTE
GYEYDGFDYWGQGTTVTVSSASTKGPSVFPLAPSSKSTSGGTAALGCLV
KDYFPEPVTVSWNSGALTSGVHTFPAVLQSSGLYSLSSVVTVPSSSLGT
QTYICNVNEEKPSNTKVDKRVEPK
(AM-VH4-T26-E40-E55)
SEQ ID NO: 10
QVQLVQSGAEVKKPGSSVKVSCKATGYTFSRYWIEWVRQAPGQGLEWM
GEILPGSGSTNYAQKFQGRVTITADESTSTAYMELSSLRSEDTAVYYC
TEGYEYDGFDYWGQGTTVTVSSASTKGPSVFPLAPSSKSTSGGTAALG
CLVKDYFPEPVTVSWNSGALTSGVHTFPAVLQSSGLYSLSSVVTVPSS
SLGTQTYICNVNIIKPSNTKVDKRVEPK

or a sequence that is >80% identical to each of the above depicted sequences respectively, and
comprises a sequence selected from the group comprising the following sequence as a VL region:

(AM-VL-C)
SEQ ID NO: 11
DVLLSQTPLSLPVSLGDQATISCRSSQSIVYSNGNTYLEWYLQKPGQSP
KLLIYRVSNRFSGVPDRFSGSGSGTDFTLKISRVEAEDLGVYYCFQGSH
IPYTFGGGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC
(AM-VL1)
SEQ ID NO: 12
DVVMTQSPLSLPVTLGQPASISCRSSQSIVYSNGNTYLNWFQQRPGQSP
RRLIYRVSNRDSGVPDRFSGSGSGTDFTLKISRVEAEDVGVYYCFQGSH
IPYTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC
(AM-VL2-E40)
SEQ ID NO: 13
DVVMTQSPLSLPVTLGQPASISCRSSQSIVYSNGNTYLEWFQQRPGQSP
RRLIYRVSNRDSGVPDRFSGSGSGTDFTLKISRVEAEDVGVYYCFQGSH
IPYTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC

or a sequence that is >80% identical to each of the above depicted sequences.

Subject matter of the present invention is an Adrenomedullin (ADM) antibody or anti-ADM antibody fragment or anti-ADM non-Ig scaffold for use in therapy or intervention in a patient infected with a Corona virus according to the present invention, wherein said antibody or fragment comprises the following sequence as a heavy chain:

SEQ ID NO: 35
QVQLVQSGAEVKKPGSSVKVSCKASGYTFSRYWIEWVRQAPGQGLEWIG
EILPGSGSTNYNQKFQGRVTITADTSTSTAYMELSSLRSEDTAVYYCTE
GYEYDGFDYWGQGTTVTVSSASTKGPSVFPLAPSSKSTSGGTAALGCLV
KDYFPEPVTVSWNSGALTSGVHTFPAVLQSSGLYSLSSVVTVPSSSLGT
QTYICNVNRKPSNTKVDKKVEPKSCDKTHTCPPCPAPELLGGPSVFLFP
PKPKDTLMISRTPEVTCVVVDVSREDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFFLYSKLTVDKSRWQQGNVFSCSVMREALHNHYTQKS
LSLSPGK

or a sequence that is >95% identical to it,
and comprises the following sequence as a light chain:

SEQ ID NO: 36
DVVLTQSPLSLPVTLGQPASISCRSSQSIVYSNGNTYLEWYLQRPGQSP
RLLIYRVSNRFSGVPDRFSGSGSGTDFTLKISRVEAEDVGVYYCFQGSH
IPYTFGGGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC

or a sequence that is >95% identical to it.

Subject matter of the present invention is an Anti-adrenomedullin (ADM) antibody or anti-ADM antibody fragment or anti-ADM non-Ig scaffold for use in therapy or intervention in a patient infected with a Corona virus according to the present invention, wherein said monoclonal antibody or antibody fragment is a humanized monoclonal antibody or humanized monoclonal antibody fragment.

Subject matter of the present invention is an Anti-adrenomedullin (ADM) antibody or anti-ADM antibody fragment or anti-ADM non-Ig scaffold for use in therapy or intervention in a patient infected with a Corona virus according to the present invention, wherein said Anti-adrenomedullin (ADM) antibody or anti-ADM antibody fragment or anti-ADM non-Ig scaffold is an monoclonal antibody and is Adrecizumab and comprises the following sequence as a heavy chain:

SEQ ID NO: 35
QVQLVQSGAEVKKPGSSVKVSCKASGYTFSRYWIEWVRQAPGQGLEWIGE
ILPGSGSTNYNQKFQGRVTITADTSTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNRKPSNTKVDKKVEPKSCDKTHTCPPCPAPELLGGPSVFLFPPKPKD
TLMISRTPEVTCVVVDVSREDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMREALHNHYTQKSLSLSPGK

and comprises the following sequence as a light chain:

SEQ ID NO: 36
DVVLTQSPLSLPVTLGQPASISCRSSQSIVYSNGNTYLEWYLQRPGQSP
RLLIYRVSNRFSGSGSGTDFTLKISRVEAEDVGVYYCFQGSHIPYTFGG
GTKLEIKRTVAAPSVFIFPPSDEQLKSGTASVVCLLNNFYPREAKVQWK
VDNALQSGNSQESVTEQDSKDSTYSLSSTLTLSKADYEKHKVYACEVTH
QGLSSPVTKSFNRGEC

or a biosimilar thereof.

A bodily fluid according to the present invention is in one particular embodiment a blood sample. A blood sample may be selected from the group comprising whole blood, serum and plasma. In a specific embodiment of the diagnostic method said sample is selected from the group comprising human citrate plasma, heparin plasma and EDTA plasma.

The biomarkers concentration like D-Dimer, like pro-Adrenomedullin or fragments thereof may be measured an immunoassay, wherein said immunoassay maybe a sandwich immunoassay, preferably a fully automated assay.

In one embodiment the assay sensitivity of said assay for ADM-Gly is able to quantify ADM-Gly of healthy subjects and is 20 pg/ml, preferably 15 pg/ml and more preferably 10 pg/ml.

In one embodiment the assay sensitivity of said assay for PAMP is able to quantify PAMP of healthy subjects and is <0.5 pmol/L, preferably <0.25 pmol/L and more preferably <0.1 pmol/L.

In one embodiment the assay sensitivity of said assay for the detection of CT-proADM is able to quantify CT-proADM of healthy subjects and is <100 pmol/L, preferably <75 pmol/L and more preferably <50 pmol/L.

In one embodiment the assay sensitivity of said assay for the detection of ADM-NH₂ is able to quantify ADM-NH₂ of healthy subjects and is <70 pg/ml, preferably <40 pg/ml and more preferably <10 pg/ml.

In one embodiment the assay sensitivity of said assay is able to quantify MR-proADM of healthy subjects and is <0.5 nmol/L, preferably <0.4 nmol/L and more preferably <0.2 nmol/L.

Further biomarkers may be measured in addition to pro-Adrenomedullin and/or fragments thereof. Said further biomarkers may be selected from the group comprising D-Dimer, procalcitonin (PCT), C-reactive protein (CRP), lactate, DPP3, penKid, NT-proBNP, white blood cell count, lymphocyte count, neutrophil count, hemoglobin, platelet count, albumin, alanine transaminase, creatinine, blood urea, lactate dehydrogenase, creatinin kinase, cardiac troponin I, prothrombin time, serum ferritin, interleukin-6 (IL-6), IL-10, IL-2, IL-7, tumor necrosis factor-α (TNF-α), granulocyte colony-stimulating factor (GCSF), IP-10, MCP-1, MW-1α.

Another embodiment of the present application relates to an anti-ADM antibody or an anti-ADM antibody fragment or anti-ADM non-Ig scaffold for use in therapy of a patient, wherein said anti-ADM antibody or anti-ADM fragment or anti-ADM non-Ig scaffold binds to the N-terminal and/or mid-regional part (amino acid 1-42) of ADM-Gly and/or ADM-NH₂:

(SEQ ID No. 23)
YRQSMNNFQGLRSFGCRFGTCTVQKLAHQIYQFTDKDKDNVA.

One embodiment of the present application relates to an Anti-ADM antibody or anti-ADM antibody fragment or anti-ADM non-Ig scaffold for use in therapy of a patient infected with corona virus, wherein said anti-ADM antibody or anti-ADM antibody fragment or anti-ADM non-Ig-protein scaffold is

a. for use in therapy of a patient for stabilizing the systemic circulation of said patient wherein said patient is in need of stabilizing the systemic circulation and exhibits a heart rate of >100 beats/min and/or <65 mm Hg mean arterial pressure and wherein stabilizing the systemic circulation means increasing the mean arterial pressure over 65 mmHg or

b. for use in the prevention of a heart rate increase to >100 beats/min and/or a mean arterial pressure decrease to <65 mm Hg in patients infected with coronavirus.

Another embodiment of the present application relates to an anti-ADM antibody or anti-ADM antibody fragment or anti-ADM non-Ig scaffold for use in therapy of patient infected with corona virus, wherein said anti-ADM antibody or anti-ADM antibody fragment or anti-ADM non-Ig-protein scaffold is for use in therapy of said patient for prevention or reduction of organ dysfunction or prevention of organ failure in said patient and wherein said organ is selected from the group comprising heart, kidney, liver, lungs, pancreas, small intestines and spleen.

In a specific embodiment of the invention said patient has been diagnosed with or is suspected of having a corona virus infection.

The term “corona virus infection” is defined as an infection with corona virus (Coronaviridae), a family of enveloped, positive-sense, single-stranded RNA viruses. The viral genome is 26-32 kilobases in length. The particles are typically decorated with large (˜20 nm), club- or petal-shaped surface projections (the “peplomers” or “spikes”), which in electron micrographs of spherical particles create an image reminiscent of the solar corona. Coronaviruses cause diseases in mammals and birds. In humans, the viruses cause respiratory infections, including the common cold, which are typically mild, though rarer forms such as SARS, MERS and COVID-19 can be lethal. The newest addition is the SARS-CoV-2.

In a specific embodiment said infection with Corona Virus is selected from the group comprising an infection with SARS-CoV-1, SARS-CoV-2, MERS-CoV, in particular SARS-CoV-2.

According to the WHO, severe acute respiratory infection (SARI) suspected of SARS-CoV-2 infection is currently defined as an acute respiratory infection (ARI) with history of fever or measured temperature ≥38° C. and cough, onset within the last ˜10 days, and requiring hospitalization. However, the absence of fever does NOT exclude viral infection.

SARS-CoV infection may present with mild, moderate, or severe illness; the latter includes severe pneumonia, ARDS, sepsis and septic shock. Early identification of those with severe manifestations (see Table 1) allows for immediate optimized supportive care treatments and safe, rapid admission (or referral) to intensive care unit according to institutional or national protocols. For those with mild illness, hospitalization may not be required unless there is concern for rapid deterioration. All patients discharged home should be instructed to return to hospital if they develop any worsening of illness.

TABLE 1
Clinical syndromes associated with 2019-nCoV infection
(according to WHO guidance) sorted by severity

| | |
|---|---|
| Uncomplicated illness | Patients with uncomplicated upper respiratory tract viral infection, may |
| | have non-specific symptoms such as fever, cough, sore throat, nasal |
| | congestion, malaise, headache, muscle pain or malaise. The elderly and |
| | immunosuppressed may present with atypical symptoms. These patients |
| | do not have any signs of dehydration, sepsis or shortness of breath. |
| Mild pneumonia | Patient with pneumonia and no signs of severe pneumonia. |
| | Child with non-severe pneumonia has cough or difficulty breathing + |
| | fast breathing: fast breathing (in breaths/min): <2 months, ≥60; 2-11 |
| | months, ≥50; 1-5 years, ≥40 and no signs of severe pneumonia. |
| Severe pneumonia | Adolescent or adult: fever or suspected respiratory infection, plus one of |
| | respiratory rate >30 breaths/min, severe respiratory distress, or |
| | SpO2 <90% on room air (adapted from [1]). |
| | Child with cough or difficulty in breathing, plus at least one of the |
| | following: central cyanosis or SpO2 <90%; severe respiratory distress |
| | (e.g. grunting, very severe chest indrawing); signs of pneumonia with a |
| | general danger sign: inability to breastfeed or drink, lethargy or |
| | unconsciousness, or convulsions. Other signs of pneumonia may be |
| | present: chest indrawing, fast breathing (in breaths/min): <2 months, ≥60; |
| | 2-11 months, ≥50; 1-5 years, ≥40.2 The diagnosis is clinical; chest |
| | imaging can exclude complications. |
| Acute Respiratory Distress | Onset: new or worsening respiratory symptoms within one week of |
| Syndrome | known clinical insult. |
| | Chest imaging (radiograph, CT scan, or lung ultrasound): bilateral |
| | opacities, not fully explained by effusions, lobar or lung collapse, or nodules. |
| | Origin of oedema: respiratory failure not fully explained by cardiac |
| | failure or fluid overload. Need objective assessment (e.g. |
| | echocardiography) to exclude hydrostatic cause of oedema if no risk |
| | factor present. |
| | Oxygenation (adults): |
| | Mild ARDS: 200 mmHg < PaO2/FiO2 ≤300 mmHg (with |
| | PEEP or CPAP ≥5 cmH2O, 7 or non-ventilated8) |
| | Moderate ARDS: 100 mmHg < PaO2/FiO2 ≤200 mmHg with |
| | PEEP ≥5 cmH2O, 7 or non-ventilated8) |
| | Severe ARDS: PaO2/FiO2 ≤100 mmHg with PEEP ≥5 |
| | cmH2O, 7 or non-ventilated8) |
| | When PaO2 is not available, SpO2/FiO2 ≤315 suggests ARDS |
| | (including in non-ventilated patients) |
| | Oxygenation (children; note OI = Oxygenation Index and OSI = |
| | Oxygenation Index using SpO2): |
| | Bilevel NIV or CPAP ≥5 cmH2O via full face mask: PaO2/FiO2 ≤300 |
| | mmHg or SpO2/FiO2 ≤264 |
| | Mild ARDS (invasively ventilated): 4 ≤ OI < 8 or 5 ≤ OSI < 7.5 |
| | Moderate ARDS (invasively ventilated): 8 ≤ OI < 16 or 7.5 ≤ OSI < 12.3 |
| | Severe ARDS (invasively ventilated): OI ≥16 or OSI ≥12.3 |
| Sepsis | Adults: life-threatening organ dysfunction caused by a dysregulated host |
| | response to suspected or proven infection, with organ dysfunction. Signs |
| | of organ dysfunction include: altered mental status, difficult or fast |
| | breathing, low oxygen saturation, reduced urine output, fast heart rate, |
| | weak pulse, cold extremities or low blood pressure, skin mottling, or |
| | laboratory evidence of coagulopathy, thrombocytopenia, acidosis, high |
| | lactate or hyperbilirubinemia. |
| | Children: suspected or proven infection and ≥2 SIRS criteria, of which |
| | one must be abnormal temperature or white blood cell count. |
| Septic shock | Adults: persisting hypotension despite volume resuscitation, requiring |
| | vasopressors to maintain MAP ≥65 mmHg and serum lactate level >2 mmol/L. |
| | Children (any hypotension (SBP <5th centile or >2 SD below normal |
| | for age) or 2-3 of the following: altered mental state; tachycardia or |
| | bradycardia (HR <90 bpm or >160 bpm in infants and HR <70 bpm or >150 |
| | bpm in children); prolonged capillary refill (>2 sec) or warm |
| | vasodilation with bounding pulses; tachypnea; mottled skin or petechial |
| | or purpuric rash; increased lactate; oliguria; hyperthermia or |
| | hypothermia. |

Oxygenation Index; OSI, Oxygenation Index using SpO₂; PaO₂, partial pressure of oxygen; PEEP, positive end-expiratory pressure; SBP, systolic blood pressure; SD, standard deviation; SIRS, systemic inflammatory response syndrome; SpO₂, oxygen saturation.
*If altitude is higher than 1000 m, then correction factor should be calculated as follows: PaO₂/FiO₂ × Barometric pressure/760.

Septic shock is a potentially fatal medical condition that occurs when sepsis, which is organ injury or damage in response to infection, leads to dangerously low blood pressure and abnormalities in cellular metabolism. The Third International Consensus Definitions for Sepsis and Septic Shock (Sepsis-3) defines septic shock as a subset of sepsis in which particularly profound circulatory, cellular, and metabolic abnormalities are associated with a greater risk of mortality than with sepsis alone. Patients with septic shock can be clinically identified by a vasopressor requirement to maintain a mean arterial pressure of 65 mm Hg or greater and serum lactate level greater than 2 mmol/L (>18 mg/dL) in the absence of hypovolemia. This combination is associated with hospital mortality rates greater than 40% (Singer et al. 2016. JAMA. 315 (8): 801-10). The primary infection is most commonly caused by bacteria, but also may be by fungi, viruses or parasites. It may be located in any part of the body, but most commonly in the lungs, brain, urinary tract, skin or abdominal organs. It can cause multiple organ dysfunction syndrome (formerly known as multiple organ failure) and death. Frequently, people with septic shock are cared for in intensive care units. It most commonly affects children, immunocompromised individuals, and the elderly, as their immune systems cannot deal with infection as effectively as those of healthy adults. The mortality rate from septic shock is approximately 25-50%.

The severity of a disease is defined as the extent of organ system derangement or physiologic decompensation for a patient. The severity may be classified into different stages using for example scoring systems.

As used herein, organ dysfunction denotes a condition or a state of health where an organ does not perform its expected function. “Organ failure” denotes an organ dysfunction to such a degree that normal homeostasis cannot be maintained without external clinical intervention. Said organ failure may pertain an organ selected from the group comprising kidney, liver, heart, lung, nervous system. By contrast, organ function represents the expected function of the respective organ within physiologic ranges. The person skilled in the art is aware of the respective function of an organ during medical examination.

Organ dysfunction may be defined by the sequential organ failure assessment score (SOFA-Score) or the components thereof. The SOFA score, previously known as the sepsis-related organ failure assessment score (Singer et al. 2016. JAMA 315(8):801-10) is used to track a person's status during the stay in an intensive care unit (ICU) to determine the extent of a person's organ function or rate of failure. The score is based on six different scores, one each for the respiratory, cardiovascular, hepatic, coagulation, renal and neurological systems each scored from 0 to 4 with an increasing score reflecting worsening organ dysfunction. The criteria for assessment of the SOFA score are described for example in Lamden et al. (for review see Lambden et al. 2019. Critical Care 23:374). SOFA score may traditionally be calculated on admission to ICU and at each 24-h period that follows. In particular, said organ dysfunction is selected from the group comprising renal decline, cardiac dysfunction, liver dysfunction or respiratory tract dysfunction.

The quick SOFA Score (quickSOFA or qSOFA) was introduced by the Sepsis-3 group in February 2016 as a simplified version of the SOFA Score as an initial way to identify patients at high risk for poor outcome with an infection (Angus et al. 2016. Critical Care Medicine. 44 (3): e113-e121). The qSOFA simplifies the SOFA score drastically by only including its 3 clinical criteria and by including “any altered mentation” instead of requiring a GCS <15. qSOFA can easily and quickly be repeated serially on patients. The score ranges from 0 to 3 points. One point is given for: low blood pressure (SBP≤100 mmHg), high respiratory rate ((≥22 breaths/min) and altered mentation (GCS≤15). The presence of 2 or more qSOFA points near the onset of infection was associated with a greater risk of death or prolonged intensive care unit stay. These are outcomes that are more common in infected patients who may be septic than those with uncomplicated infection. Based upon these findings, the Third International Consensus Definitions for Sepsis recommends qSOFA as a simple prompt to identify infected patients outside the ICU who are likely to be septic (Seymour et al. 2016. JAMA 315(8): 762-774).

A life-threatening deterioration is defined as a condition of a patient associated with a high risk of death that involves vital organ system failure including central nervous system failure, renal failure, hepatic failure, metabolic failure or respiratory failure.

An adverse event is defined as death, organ dysfunction or shock, ARDS and ALI (Acute Lung Injury).

In the present invention, the term “prognosis” or “prognosing” denotes a prediction of how a subject's (e.g., a patient's) medical condition will progress. This may include an estimation of the chance of recovery or the chance of an adverse event or outcome for said subject.

Said prognosis of an adverse event including death may be made for a defined period of time, e.g. up to 1 year, preferably up to 6 months, more preferred up to 3 months, more preferred up to 90 days, more preferred up to 60 days, more preferred up to 28 days, more preferred up to 14 days, more preferred up to 7 days, more preferred up to 3 days.

In a specific embodiment said prognosis of an adverse event including death is made for a period of time up to 28 days.

The term “therapy monitoring” in the context of the present invention refers to the monitoring and/or adjustment of a therapeutic treatment of said patient, for example by obtaining feedback on the efficacy of the therapy.

As used herein, the term “therapy guidance” refers to application of certain therapies or medical interventions based on the value of one or more biomarkers and/or clinical parameter and/or clinical scores.

Said clinical parameter or clinical scores are selected from the group comprising history of hypotension, vasopressor requirement, intubation, mechanical ventilation, Horovitz index, SOFA score, quick SOFA score.

The term “therapy stratification” in particular relates to grouping or classifying patients into different groups, such as therapy groups that receive or do not receive therapeutic measures depending on their classification.

Said therapy or intervention may be selected from the group comprising drug therapy, non-invasive ventilation, mechanical ventilation, extracorporeal membrane oxygenation (ECMO), dialysis or renal replacement therapy.

Non-invasive ventilation is the use of breathing support administered through a face mask, nasal mask, or a helmet. Air, usually with added oxygen, is given through the mask under positive pressure.

Mechanical ventilation or assisted ventilation, is the medical term for artificial ventilation where mechanical means are used to assist or replace spontaneous breathing. This may involve a machine called a ventilator, or the breathing may be assisted manually by a suitably qualified professional, such as an anesthesiologist, respiratory therapist (RT), Registered Nurse, or paramedic, by compressing a bag valve mask device. Mechanical ventilation is termed “invasive” if it involves any instrument inside the trachea through the mouth, such as an endotracheal tube or the skin, such as a tracheostomy tube. Face or nasal masks are used for non-invasive ventilation in appropriately selected conscious patients.

Extracorporeal membrane oxygenation (ECMO), also known as extracorporeal life support (ECLS), is an extracorporeal technique of providing prolonged cardiac and respiratory support to persons whose heart and lungs are unable to provide an adequate amount of gas exchange or perfusion to sustain life. The technology for ECMO is largely derived from cardiopulmonary bypass, which provides shorter-term support with arrested native circulation. ECMO works by removing blood from the person's body and artificially removing carbon dioxide from, and adding oxygen to, the patient's red blood cells. Generally, it is used either post-cardiopulmonary bypass or in late-stage treatment of a person with profound heart and/or lung failure, although it is now seeing use as a treatment for cardiac arrest in certain centers, allowing treatment of the underlying cause of arrest while circulation and oxygenation are supported. ECMO is also used to support patients with the acute viral pneumonia associated with COVID-19 in cases where artificial ventilation is not sufficient to sustain blood oxygenation levels.

Said drug therapy may be selected from the group comprising anti-ADM antibodies, anti-ADM antibody fragments, anti-ADM non-Ig scaffolds, antiviral drugs, immunoglobulin from cured patients with COVID-19 pneumonia, neutralizing monoclonal antibodies targeting coronaviruses, immunoenhancers, camostat mesylate, coronaviral protease inhibitors (e.g. chymotrypsin-like inhibitors, papain-like protease inhibitors), spike (S) protein-angiotensin-converting enzyme-2 (ACE2) blockers (e.g. chloroquine, hydroxychloroquine, emodin, promazine), angiotensin-receptor-agonist and/or a precursor thereof.

Said neutralizing monoclonal antibodies targeting SARS-CoV and MERS-CoV may be selected from the group as summarized in Shanmugaraj et al. (Shanmugaraj et al. 2020. Asian Pac J. allergy Immunol 38: 10-18).

Said antiviral drugs may be selected from the group comprising Lopinavir, Ritonavir, Remdesivir, Nafamostat, Ribavirin, Oseltamivir, Penciclovir, Acyclovir, Ganciclovir, Favipiravir, Nitazoxanide, Nelfinavir, arbidol.

Said immunoenhancers may be selected from the group comprising interferons, intravenous gammaglobulin, thymosin α-1, levamisole, non-immunosuppressive derivatives of cyclosporin-A.

In one embodiment said Angiotensin-Receptor-Agonist and/or a precursor thereof is selected from the group comprising Angiotensin I, Angiotensin II, angiotensin III, angiotensin W.

The Horowitz index (synonyms: oxygenation after Horowitz, Horowitz quotient, P/F ratio) is a ratio used to assess lung function in patients, particularly those on ventilators. It is useful for evaluating the extent of damage to the lungs. The Horowitz index is defined as the ratio of partial pressure of oxygen in blood (PaO2), in millimeters of mercury, and the fraction of oxygen in the inhaled air (FIO2)—the PaO2/FiO2 ratio. In healthy lungs the Horowitz index depends on age and usually falls between 350 and 450. A value below 300 is the threshold for mild lung injury, and 200 is indicative of a moderately severe lung injury. A value below 100 as a criterion for a severe injury. The Horowitz index plays a major role in the diagnosis of acute respiratory distress syndrome (ARDS). Three severities of ARDS are categorized based on the degree of hypoxemia using the Horowitz index, according to the Berlin definition (Matthay et al. 2012. J Clin Invest. 122(8): 2731-2740).

Acute respiratory distress syndrome (ARDS) is a type of respiratory failure characterized by rapid onset of widespread inflammation in the lungs. Symptoms include shortness of breath, rapid breathing, and bluish skin coloration. For those who survive, a decreased quality of life is common. Causes may include sepsis, pancreatitis, trauma, pneumonia, and aspiration. The underlying mechanism involves diffuse injury to cells which form the barrier of the microscopic air sacs of the lungs, surfactant dysfunction, activation of the immune system, and dysfunction of the body's regulation of blood clotting. In effect, ARDS impairs the lungs' ability to exchange oxygen and carbon dioxide. Diagnosis is based on a PaO₂/FiO₂ ratio (ratio of partial pressure arterial oxygen and fraction of inspired oxygen) of less than 300 mm Hg despite a positive end-expiratory pressure (PEEP) of more than 5 cm H₂O. The primary treatment involves mechanical ventilation together with treatments directed at the underlying cause. Ventilation strategies include using low volumes and low pressures. If oxygenation remains insufficient, lung recruitment maneuvers and neuromuscular blockers may be used. If this is insufficient, extracorporeal membrane oxygenation (ECMO) may be an option. The syndrome is associated with a death rate between 35 and 50%.

The term “patient” as used herein refers to a living human or non-human organism that is receiving medical care or that should receive medical care due to a disease. This includes persons with no defined illness who are being investigated for signs of pathology. Thus, the methods and assays described herein are applicable to both, human and veterinary disease.

The term “patient management” in the context of the present invention refers to:
the decision for admission to hospital or intensive care unit,
the decision for relocation of the patient to a specialized hospital or a specialized hospital unit,
the evaluation for an early discharge from the intensive care unit or hospital,
the allocation of resources (e.g., physician and/or nursing staff, diagnostics, therapeutics),
the decision on therapeutic treatment.

Threshold levels can be obtained for instance from a Kaplan-Meier analysis, where the occurrence of a disease is correlated with the quartiles of the biomarker in the population. According to this analysis, subjects with biomarker levels above the 75th percentile have a significantly increased risk for getting the diseases according to the invention. This result is further supported by Cox regression analysis with full adjustment for classical risk factors: The highest quartile versus all other subjects is highly significantly associated with increased risk for getting a disease according to the invention.

Other preferred cut-off values are for instance the 90th, 95th or 99th percentile of a normal population. By using a higher percentile than the 75th percentile, one reduces the number of false positive subjects identified, but one might miss to identify subjects, who are at moderate, albeit still increased risk. Thus, one might adopt the cut-off value depending on whether it is considered more appropriate to identify most of the subjects at risk at the expense of also identifying “false positives”, or whether it is considered more appropriate to identify mainly the subjects at high risk at the expense of missing several subjects at moderate risk.

The above-mentioned threshold values might be different in other assays, if these have been calibrated differently from the assay system used in the present invention. Therefore, the above-mentioned threshold shall apply for such differently calibrated assays accordingly, taking into account the differences in calibration. One possibility of quantifying the difference in calibration is a method comparison analysis (correlation) of the assay in question (e.g. bio-ADM assay) with the respective biomarker assay used in the present invention by measuring the respective biomarker (e.g. bio-ADM) in samples using both methods. Another possibility is to determine with the assay in question, given this test has sufficient analytical sensitivity, the median biomarker level of a representative normal population, compare results with the median biomarker levels as described in the literature (e.g. Weber et al. 2017. JALM 2(2): 222-233) and recalculate the calibration based on the difference obtained by this comparison. With the calibration used in the present invention, samples from normal (healthy) subjects have been measured: median plasma bio-ADM (mature ADM-NH₂) was 13.7 pg/ml (inter quartile range [IQR] 9.6-18.7 pg/mL) (Weber et al. 2017. JALM 2(2): 222-233).

Throughout the specification the “antibodies”, or “antibody fragments” or “non-Ig scaffolds” in accordance with the invention are capable to bind ADM, and thus are directed against ADM, and thus can be referred to as “anti-ADM antibodies”, “anti-ADM antibody fragments”, or “anti-ADM non-Ig scaffolds”.

Mature ADM, bio-ADM and ADM-NH₂ is used synonymously throughout this application and is a molecule according to SEQ ID No.: 20.

In a specific embodiment of the diagnostic method, said binder exhibits a binding affinity to pro-Adrenomedullin or a fragment thereof (which is not ADM-NH₂ according to SEQ ID No.: 20) and ADM-NH₂ of at least 10⁷ M⁻¹, preferred 10⁸ M⁻¹, preferred affinity is greater than 10⁹ M⁻¹, most preferred greater than 10¹⁰ M⁻¹. A person skilled in the art knows that it may be considered to compensate lower affinity by applying a higher dose of compounds and this measure would not lead out-of-the-scope of the invention.

To determine the affinity of the antibodies to Adrenomedullin, the kinetics of binding of Adrenomedullin to immobilized antibody was determined by means of label-free surface plasmon resonance using a Biacore 2000 system (GE Healthcare Europe GmbH, Freiburg, Germany). Reversible immobilization of the antibodies was performed using an anti-mouse Fc antibody covalently coupled in high density to a CM5 sensor surface according to the manufacturer's instructions (mouse antibody capture kit; GE Healthcare), (Lorenz et al. 2011. Antimicrob Agents Chemother. 55 (1): 165-173).

In a specific embodiment of the diagnostic method, an assay is used for determining the level of pro-Adrenomedullin or a fragment thereof and ADM-NH₂, wherein said level of pro-Adrenomedullin or a fragment thereof is selected from the group consisting of PAMP (SEQ ID No. 32), MR-proADM (SEQ ID No. 33), ADM-Gly (SEQ ID No. 21) and CT-proADM (SEQ ID No. 34) and wherein such assay is a sandwich assay, preferably a fully automated assay.

In one embodiment of the invention it may be a so-called POC-test (point-of-care) that is a test technology, which allows performing the test within less than 1 hour near the patient without the requirement of a fully automated assay system. One example for this technology is the immunochromatographic test technology.

In one embodiment of the diagnostic method such an assay is a sandwich immunoassay using any kind of detection technology including but not restricted to enzyme label, chemiluminescence label, electrochemiluminescence label, preferably a fully automated assay. In one embodiment of the diagnostic method such an assay is an enzyme labeled sandwich assay. Examples of automated or fully automated assay comprise assays that may be used for one of the following systems: Roche Elecsys®, Abbott Architect®, Siemens Centauer®, Brahms Kryptor®, BiomerieuxVidas®, Alere Triage®.

A variety of immunoassays are known and may be used for the assays and methods of the present invention, these include: radioimmunoassays (“RIA”), homogeneous enzyme-multiplied immunoassays (“EMIT”), enzyme linked immunoadsorbent assays (“ELISA”), apoenzyme reactivation immunoassay (“ARIS”), dipstick immunoassays and immuno-chromatography assays.

In a specific embodiment of the diagnostic method, at least one of said two binders is labeled in order to be detected.

Monospecific means that said antibody or antibody fragment or non-Ig scaffold binds to one specific region encompassing at least 4 amino acids within the target ADM. Monospecific antibodies or fragments or non-Ig scaffolds according to the invention are antibodies or fragments or non-Ig scaffolds that all have affinity for the same antigen. Monoclonal antibodies are monospecific, but monospecific antibodies may also be produced by other means than producing them from a common germ cell.

Said anti-ADM antibody or antibody fragment binding to ADM or non-Ig scaffold binding to ADM may be a non-neutralizing anti-ADM antibody or antibody fragment binding to ADM or non-Ig scaffold binding to ADM.

An antibody or fragment according to the present invention is a protein including one or more polypeptides substantially encoded by immunoglobulin genes that specifically binds an antigen. The recognized immunoglobulin genes include the kappa, lambda, alpha (IgA), gamma (IgG₁, IgG₂, IgG₃, IgG₄), delta (IgD), epsilon (IgE) and mu (IgM) constant region genes, as well as the myriad immunoglobulin variable region genes. Full-length immunoglobulin light chains are generally about 25 Kd or 214 amino acids in length.

Full-length immunoglobulin heavy chains are generally about 50 Kd or 446 amino acid in length. Light chains are encoded by a variable region gene at the NH₂-terminus (about 110 amino acids in length) and a kappa or lambda constant region gene at the COOH-terminus. Heavy chains are similarly encoded by a variable region gene (about 116 amino acids in length) and one of the other constant region genes.

The basic structural unit of an antibody is generally a tetramer that consists of two identical pairs of immunoglobulin chains, each pair having one light and one heavy chain. In each pair, the light and heavy chain variable regions bind to an antigen, and the constant regions mediate effector functions. Immunoglobulins also exist in a variety of other forms including, for example, Fv, Fab, and (Fab′)₂, as well as bifunctional hybrid antibodies and single chains (e.g., Lanzavecchia et al. 1987. Eur. J. Immunol. 17:105; Huston et al. 1988. Proc. Natl. Acad. Sci. U.S.A., 85:5879-5883; Bird et al. 1988. Science 242:423-426; Hood et al. 1984, Immunology, Benjamin, N.Y., 2nd ed; Hunkapiller and Hood 1986. Nature 323:15-16). An immunoglobulin light or heavy chain variable region includes a framework region interrupted by three hypervariable regions, also called complementarity determining regions (CDR's) (see, Sequences of Proteins of Immunological Interest, E. Kabat et al. 1983, U.S. Department of Health and Human Services). As noted above, the CDRs are primarily responsible for binding to an epitope of an antigen. An immune complex is an antibody, such as a monoclonal antibody, chimeric antibody, humanized antibody or human antibody, or functional antibody fragment, specifically bound to the antigen.

Chimeric antibodies are antibodies whose light and heavy chain genes have been constructed, typically by genetic engineering, from immunoglobulin variable and constant region genes belonging to different species. For example, the variable segments of the genes from a mouse monoclonal antibody can be joined to human constant segments, such as kappa and gamma 1 or gamma 3. In one example, a therapeutic chimeric antibody is thus a hybrid protein composed of the variable or antigen-binding domain from a mouse antibody and the constant or effector domain from a human antibody, although other mammalian species can be used, or the variable region can be produced by molecular techniques. Methods of making chimeric antibodies are well known in the art, e.g., see U.S. Pat. No. 5,807,715. A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions, which have substantially no effect on antigen binding or other immunoglobulin functions. Exemplary conservative substitutions are those such as gly, ala; val, ile, leu; asp, glu; asn, gln; ser, thr; lys, arg; and phe, tyr. Humanized immunoglobulins can be constructed by means of genetic engineering (e.g., see U.S. Pat. No. 5,585,089). A human antibody is an antibody wherein the light and heavy chain genes are of human origin. Human antibodies can be generated using methods known in the art. Human antibodies can be produced by immortalizing a human B cell secreting the antibody of interest. Immortalization can be accomplished, for example, by EBV infection or by fusing a human B cell with a myeloma or hybridoma cell to produce a trioma cell. Human antibodies can also be produced by phage display methods (see, e.g. WO91/17271; WO92/001047; WO92/20791), or selected from a human combinatorial monoclonal antibody library (see the Morphosys website). Human antibodies can also be prepared by using transgenic animals carrying a human immunoglobulin gene (for example, see WO93/12227; WO 91/10741).

Thus, the anti-ADM antibody may have the formats known in the art. Examples are human antibodies, monoclonal antibodies, humanized antibodies, chimeric antibodies, CDR-grafted antibodies. In a preferred embodiment antibodies according to the present invention are recombinantly produced antibodies as e.g. IgG, a typical full-length immunoglobulin, or antibody fragments containing at least the F-variable domain of heavy and/or light chain as e.g. chemically coupled antibodies (fragment antigen binding) including but not limited to Fab-fragments including Fab minibodies, single chain Fab antibody, monovalent Fab antibody with epitope tags, e.g. Fab-V5Sx2; bivalent Fab (mini-antibody) dimerized with the CH3 domain; bivalent Fab or multivalent Fab, e.g. formed via multimerization with the aid of a heterologous domain, e.g. via dimerization of dHLX domains, e.g. Fab-dHLX-FSx2; F(ab′)₂-fragments, scFv-fragments, multimerized multivalent or/and multispecific scFv-fragments, bivalent and/or bispecific diabodies, BITE® (bispecific T-cell engager), trifunctional antibodies, polyvalent antibodies, e.g. from a different class than G; single-domain antibodies, e.g. nanobodies derived from camelid or fish immunoglobulines and numerous others.

In addition to anti-ADM antibodies other biopolymer scaffolds are well known in the art to complex a target molecule and have been used for the generation of highly target specific biopolymers. Examples are aptamers, spiegelmers, anticalins and conotoxins. For illustration of antibody formats please see FIGS. 1A, 1B and 1C.

In a preferred embodiment the anti-ADM antibody format is selected from the group comprising Fv fragment, scFv fragment, Fab fragment, scFab fragment, F(ab)₂ fragment and scFv-Fc Fusion protein. In another preferred embodiment the antibody format is selected from the group comprising scFab fragment, Fab fragment, scFv fragment and bioavailability optimized conjugates thereof, such as PEGylated fragments. One of the most preferred formats is the scFab format.

Non-Ig scaffolds may be protein scaffolds and may be used as antibody mimics as they are capable to bind to ligands or antigens. Non-Ig scaffolds may be selected from the group comprising tetranectin-based non-Ig scaffolds (e.g. described in US 2010/0028995), fibronectin scaffolds (e.g. described in EP 1 266 025; lipocalin-based scaffolds (e.g. described in WO 2011/154420); ubiquitin scaffolds (e.g. described in WO 2011/073214), transferrin scaffolds (e.g. described in US 2004/0023334), protein A scaffolds (e.g. described in EP 2 231 860), ankyrin repeat based scaffolds (e.g. described in WO 2010/060748), microproteins preferably microproteins forming a cysteine knot) scaffolds (e.g. described in EP 2314308), Fyn SH3 domain based scaffolds (e.g. described in WO 2011/023685) EGFR-A-domain based scaffolds (e.g. described in WO 2005/040229) and Kunitz domain based scaffolds (e.g. described in EP 1 941 867).

In one embodiment of the invention anti-ADM antibodies according to the present invention may be produced as outlined in Example 1 by synthesizing fragments of ADM as antigens. Thereafter, binder to said fragments are identified using the below described methods or other methods as known in the art.

Humanization of murine antibodies may be conducted according to the following procedure: For humanization of an antibody of murine origin the antibody sequence is analyzed for structural interaction of framework regions (FR) with the complementary determining regions (CDR) and the antigen. Based on structural modelling an appropriate FR of human origin is selected and the murine CDR sequences are transplanted into the human FR. Variations in the amino acid sequence of the CDRs or FRs may be introduced to regain structural interactions, which were abolished by the species switch for the FR sequences. This recovery of structural interactions may be achieved by random approach using phage display libraries or via directed approach guided by molecular modelling (Almagro and Fransson 2008. Front Biosci. 13:1619-33).

In another embodiment, the anti-ADM antibody, anti-ADM antibody fragment, or anti-ADM non-Ig scaffold is a full-length antibody, antibody fragment, or non-Ig scaffold.

In a embodiment, the anti-ADM antibody or anti-ADM antibody fragment or anti-ADM non-Ig scaffold is directed to and can bind to an epitope of preferably at least 4 or at least 5 amino acids in length of the N-terminal and/or mid-regional part (amino acid 1-42) of ADM-Gly and/or ADM-NH₂:

(SEQ ID No. 23)
YRQSMNNFQGLRSFGCRFGTCTVQKLAHQIYQFTDKDKDNVA.

An epitope, also known as antigenic determinant, is the part of an antigen (e.g., peptide or protein) that is recognized by the immune system, specifically by antibodies. For example, the epitope is the specific piece of the antigen to which an antibody binds. The part of an antibody that binds to the epitope is called a paratope. The epitopes of protein antigens are divided into two categories: conformational epitopes and linear epitopes, based on their structure and interaction with the paratope.

A linear or a sequential epitope is an epitope that is recognized by antibodies by its linear sequence of amino acids, or primary structure and is formed by the 3-D conformation adopted by the interaction of contiguous amino acid residues. Conformational and linear epitopes interact with the paratope based on the 3-D conformation adopted by the epitope, which is determined by the surface features of the involved epitope residues and the shape or tertiary structure of other segments of the antigen. A conformational epitope is formed by the 3-D conformation adopted by the interaction of discontinuous amino acid residues.

In one specific embodiment of the invention the anti-ADM antibody or anti-ADM antibody fragment or anti-ADM non-Ig scaffold is directed to and can bind to preferably at least 4, or at least 5 amino acids within the N-terminal part (amino acid 1-21) of ADM-Gly and/or ADM-NH₂:

(SEQ ID No.: 14)
YRQSMNNFQGLRSFGCRFGTC.

In another preferred embodiment said anti-ADM-antibody or anti-ADM antibody fragment or anti-ADM non-Ig scaffold is directed to and can bind to preferably at least 4, or at least 5 amino acids within the N-terminal part (amino acid 1-14) of ADM-Gly and/or ADM-NH₂:

(SEQ ID No.: 25)
YRQSMNNFQGLRSF.

In another embodiment said anti-ADM-antibody or anti-ADM antibody fragment or anti-ADM non-Ig scaffold is directed to and can bind to preferably at least 4, or at least 5 amino acids within the N-terminal part (amino acid 1-10) of ADM-Gly and/or ADM-NH₂: YRQSMNNFQG (SEQ ID No.: 26).

In a very specific embodiment said anti-ADM-antibody or anti-ADM antibody fragment or anti-ADM non-Ig scaffold is directed to and can bind to preferably at least 4, or at least 5 amino acids within the N-terminal part (amino acid 1-6) of ADM-Gly and/or ADM-NH₂: YRQSMN (SEQ ID No.: 27) and needs the free N-terminus (amino acid 1) of ADM and/or ADM-Gly for binding.

In another very specific embodiment of the invention the anti-ADM antibody or anti-adrenomedullin antibody fragment or anti-ADM non-Ig scaffold recognizes and binds to the N-terminal end (amino acid 1) of ADM-Gly and/or ADM-NH₂. N-terminal end means that the amino acid 1, that is “Y” of SEQ ID No. 14, 20, 22, 23, 25, 26, 27 is mandatory for antibody binding. The antibody or fragment or scaffold would neither bind N-terminal extended nor N-terminal modified ADM nor N-terminal degraded ADM-Gly and/or ADM-NH₂. This means that said anti-ADM-antibody or anti-ADM antibody fragment or anti-ADM non-Ig scaffold binds only to a region within the sequence of ADM-Gly and/or ADM-NH₂ if the N-terminal end of ADM is free. The anti-ADM antibody or anti-ADM antibody fragment or non-Ig scaffold would not bind to a region within the sequence of ADM-Gly and/or ADM-NH₂ if said sequence is e.g. comprised within pro-ADM.

For the sake of clarity, the numbers in brackets for specific regions of ADM like “N-terminal part (amino acid 1-21)” is understood by a person skilled in the art that the N-terminal part of ADM consists of amino acids 1-21 of the ADM-Gly and/or ADM-NH₂ sequence.

In another specific embodiment pursuant to the invention the herein provided anti-ADM antibody or anti-ADM antibody fragment or anti-ADM non-Ig scaffold does not bind to the C-terminal portion of ADM, i.e. the aa 43-52 of ADM (SEQ ID No.: 24).

In one specific embodiment it is preferred to use an anti-ADM antibody or an anti-adrenomedullin antibody fragment or anti-ADM non-Ig scaffold according to the present invention, wherein said anti-adrenomedullin antibody or said anti-adrenomedullin antibody fragment or non-Ig scaffold leads to an increase of the ADM-NH₂ level or ADM-NH₂ immunoreactivity in serum, blood, plasma of at least 10%, preferably at least 50%, more preferably >50%, most preferably >100%.

An assay that may be used for the determination of the half-life (half retention time) of adrenomedullin in serum, blood, plasma is described in Example 3.

In a specific embodiment of the invention the antibody is a monoclonal antibody or a fragment thereof. In one embodiment of the invention the anti-ADM antibody or the anti-ADM antibody fragment is a human or humanized antibody or derived therefrom. In one specific embodiment one or more (murine) CDR's are grafted into a human antibody or antibody fragment (“humanization”).

Subject matter of the present invention in one aspect is a humanized CDR-grafted antibody or antibody fragment thereof, wherein said antibody recognizes or binds to the N-terminal part of ADM-Gly and/or ADM-NH₂ for therapy or intervention in a patient infected with a Corona virus, wherein the humanized CDR-grafted antibody or antibody fragment thereof comprises an antibody heavy chain (H chain) comprising:

SEQ ID No.: 1
GYTFSRYW
SEQ ID No.: 2
ILPGSGST
and/or
SEQ ID No.: 3
TEGYEYDGFDY

and/or further comprises an antibody light chain (L chain) comprising:

SEQ ID No.: 4
QSIVYSNGNTY

SEQUENCE “RVS” (not part of the Sequencing Listing): RVS

and/or

SEQ ID No.: 5
FQGSHIPYT.

One specific embodiment of the invention is a humanized and/or human monoclonal antibody or an antibody fragment thereof, wherein said antibody recognizes or binds to the N-terminal part (amino acid 1-21) of ADM-Gly and/or ADM-NH₂: YRQSMNNFQGLRSFGCRFGTC (SEQ ID No.: 14) for therapy or intervention in a patient infected with a Corona virus wherein the heavy chain comprises at least one CDR selected from the group comprising:

SEQ ID No.: 1
GYTFSRYW
SEQ ID No.: 2
ILPGSGST
SEQ ID No.: 3
TEGYEYDGFDY

and wherein the light chain comprises at least one CDR selected from the group comprising:

SEQ ID No.: 4
QSIVYSNGNTY

SEQUENCE “RVS” (not part of the Sequencing Listing): RVS

SEQ ID No.: 5
FQGSHIPYT.

In a more specific embodiment of the invention subject matter of the invention is a humanized and/or human monoclonal antibody or antibody fragment thereof, wherein said antibody recognizes or binds to the N-terminal part (amino acid 1-21) of ADM-Gly and/or ADM-NH₂: YRQSMNNFQGLRSFGCRFGTC (SEQ ID No.: 14) for therapy or intervention in a patient infected with a Corona virus wherein the heavy chain comprises the sequences:

SEQ ID No.: 1
GYTFSRYW
SEQ ID No.: 2
ILPGSGST
SEQ ID No.: 3
TEGYEYDGFDY

and wherein the light chain comprises the sequences:

SEQ ID No.: 4
QSIVYSNGNTY

SEQUENCE “RVS” (not part of the Sequencing Listing): RVS

SEQ ID No.: 5
FQGSHIPYT.

In a very specific embodiment, the anti-ADM antibody has a sequence selected from the group comprising: SEQ ID No. 6, 7, 8, 9, 10, 11, 12, 13, 35 and 36.

The anti-ADM antibody or anti-ADM antibody fragment or anti-ADM non-Ig scaffold according to the present invention exhibits an affinity towards human ADM-Gly and/or ADM-NH₂ in such that affinity constant is greater than 10⁻⁷ M, preferred 10⁻⁸ M, preferred affinity is greater than 10″ ⁹ M, most preferred higher than 10⁻¹⁰ M. A person skilled in the art knows that it may be considered to compensate lower affinity by applying a higher dose of compounds and this measure would not lead out-of-the-scope of the invention. The affinity constants may be determined according to the method as described in Example 1.

Subject matter of the present invention is a human or humanized monoclonal antibody or fragment that binds to ADM-Gly and/or ADM-NH₂, wherein said antibody or fragment binds to the N-terminal (amino acid 1-21) of ADM-Gly and/or ADM-NH₂: YRQSMNNFQGLRSFGCRFGTC (SEQ ID No.: 14) for therapy or intervention in a patient infected with a Corona virus, wherein said antibody or fragment comprises a sequence selected from the group comprising:

(AM-VH-C)
SEQ ID No.: 6
QVQLQQSGAELMKPGASVKISCKATGYTFSRYWIEWVKQRPGHGLEWIGE
ILPGSGSTNYNEKFKGKATITADTSSNTAYMQLSSLTSEDSAVYYCTEGY
EYDGFDYWGQGTTLTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPK
(AM-VH1)
SEQ ID No.: 7
QVQLVQSGAEVKKPGSSVKVSCKASGYTFSRYWISWVRQAPGQGLEWMGR
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPK
(AM-VH2-E40)
SEQ ID No.: 8
QVQLVQSGAEVKKPGSSVKVSCKASGYTFSRYWIEWVRQAPGQGLEWMGR
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNKPSNTKVDKRVEPK
(AM-VH3-T26-E55)
SEQ ID No.: 9
QVQLVQSGAEVKKPGSSVKVSCKATGYTFSRYWISWVRQAPGQGLEWMGE
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPK
(AM-VH4-T26-E40-E55)
SEQ ID No.: 10
QVQLVQSGAEVKKPGSSVKVSCKATGYTFSRYWIEWVRQAPGQGLEWMGE
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPK
(AM-VL-C)
SEQ ID No.: 11
DVLLSQTPLSLPVSLGDQATISCRSSQSIVYSNGNTYLEWYLQKPGQSPK
LLIYRVSNRFSGVPDRFSGSGSGTDFTLKISRVEAEDLGVYYCFQGSHIP
YTFGGGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
(AM-VL1)
SEQ ID No.: 12
DVVMTQSPLSLPVTLGQPASISCRSSQSIVYSNGNTYLNWFQQRPGQSPR
RLIYRVSNRDSGVPDRFSGSGSGTDFTLKISRVEAEDVGVYYCFQGSHIP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
(AM-VL2-E40)
SEQ ID No.: 13
DVVMTQSPLSLPVTLGQPASISCRSSQSIVYSNGNTYLEWFQQRPGQSPR
RLIYRVSNRDSGVPDRFSGSGSGTDFTLKISRVEAEDVGVYYCFQGSHIP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
(heavy chain HAM8101)
SEQ ID No.: 35
QVQLVQSGAEVKKPGSSVKVSCKASGYTFSRYWIEWVRQAPGQGLEWIGE
ILPGSGSTNYNQKFQGRVTITADTSTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSREDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFScSVMHEALHNHYTQKSLSLSPGK
(light chain HAM 8101)
SEQ ID No.: 36
DVVLTQSPLSLPVTLGQPASISCRSSQSIVYSNGNTYLEWYLQRPGQSPR
LLIYRVSNRFSGVPDRFSGSGSGTDFTLKISRVEAEDVGVYYCFQGSHIP
YTFGGGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

Subject matter of the present invention is further a human and/or humanized monoclonal antibody or fragment that binds to ADM-Gly and/or ADM-NH₂, wherein said antibody or fragment binds to the N-terminal part (amino acid 1-21) of ADM-Gly and/or ADM-NH₂: YRQSMNNFQGLRSFGCRFGTC (SEQ ID No.: 14) for therapy or intervention in a patient infected with a Corona virus, wherein said antibody or fragment comprises the following sequence as a heavy chain:

SEQ ID No.: 35
QVQLVQSGAEVKKPGSSVKVSCKASGYTFSRYWIEWVRQAPGQGLEWIGE
ILPGSGSTNYNQKFQGRVTITADTSTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFScSVMHEALHNHYTQKSLSLSPGK

and comprises the following sequence as a light chain:

SEQ ID No.: 36
DVVLTQSPLSLPVTLGQPASISCRSSQSIVYSNGNTYLEWYLQRPGQSPR
LLIYRVSNRFSGVPDRFSGSGSGTDFTLKISRVEAEDVGVYYCFQGSHIP
YTFGGGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

In a specific embodiment of the invention the antibody comprises the following sequence as a heavy chain:

SEQ ID No.: 35
QVQLVQSGAEVKKPGSSVKVSCKASGYTFSRYWIEWVRQAPGQGLEWIGE
ILPGSGSTNYNQKFQGRVTITADTSTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK

or a sequence that is >95% identical to it, preferably >98%, preferably >99% and comprises the following sequence as a light chain:

SEQ ID No.: 36
DVVLTQSPLSLPVTLGQPASISCRSSQSIVYSNGNTYLEWYLQRPGQSPR
LLIYRVSNRFSGVPDRFSGSGSGTDFTLKISRVEAEDVGVYYCFQGSHIP
YTFGGGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

or a sequence that is >95% identical to it, preferably >98%, preferably >99%.

To assess the identity between two amino acid sequences, a pairwise alignment is performed. Identity defines the percentage of amino acids with a direct match in the alignment.

The term “pharmaceutical formulation” means a pharmaceutical ingredient in combination with at least one pharmaceutically acceptable excipient, which is in such form as to permit the biological activity of a pharmaceutical ingredient contained therein to be effective, and which contains no additional components which are unacceptably toxic to a subject to which the formulation would be administered. The term “pharmaceutical ingredient” means a therapeutic composition which can be optionally combined with pharmaceutically acceptable excipients to provide a pharmaceutical formulation or dosage form.

Subject matter of the present invention is a pharmaceutical formulation for use in therapy or intervention in a patient infected with a Corona virus in a patient comprising an antibody or fragment or scaffold according to the present invention.

Subject matter of the present invention is a pharmaceutical formulation for use in therapy or intervention in a patient infected with a Corona virus according to the present invention wherein said pharmaceutical formulation is a solution, preferably a ready-to-use solution.

Subject matter of the present invention is a pharmaceutical formulation for use in therapy or intervention in a patient infected with a Corona virus according to the present invention wherein said pharmaceutical formulation is in a freeze-dried state.

Subject matter of the present invention is a pharmaceutical formulation for use in therapy or intervention in a patient infected with a Corona virus according to the present invention, wherein said pharmaceutical formulation is administered intra-muscular.

Subject matter of the present invention is a pharmaceutical formulation for use in therapy or intervention in a patient infected with a Corona virus according to the present invention, wherein said pharmaceutical formulation is administered intra-vascular.

Subject matter of the present invention is a pharmaceutical formulation for use in therapy or intervention in a patient infected with a Corona virus according to the present invention, wherein said pharmaceutical formulation is administered via infusion.

Subject matter of the present invention is a pharmaceutical formulation for use in therapy or intervention in a patient infected with a Corona virus according to the present invention, wherein said pharmaceutical formulation is to be administered systemically.

### EMBODIMENTS

1. A method for (a) diagnosing or predicting the risk of life-threatening deterioration or an adverse event or (b) diagnosing or prognosing the severity or (c) predicting or monitoring the success of a therapy or intervention or (d) therapy guidance or therapy stratification or (e) patient management in a patient infected with a Corona virus, the method comprising:
determining the level of pro-Adrenomedullin (SEQ ID No. 31) or fragment thereof in a sample of bodily fluid of said patient,
comparing said level of pro-Adrenomedullin or fragment thereof to a pre-determined threshold or to a previously measured level of pro-Adrenomedullin or fragment thereof, and
correlating said level of pro-Adrenomedullin or fragment thereof with the risk of life-threatening deterioration or an adverse event, or
correlating said level of pro-Adrenomedullin or fragment thereof with the severity, or
correlating said level of pro-Adrenomedullin or fragment thereof with the success of a therapy or intervention, or
correlating said level of pro-Adrenomedullin or fragment thereof with a certain therapy or intervention, or
correlating said level of pro-Adrenomedullin or fragment thereof with the management of said patient,

wherein said pro-Adrenomedullin or fragment thereof is selected from the group consisting of PAMP (SEQ ID No. 32), MR-proADM (SEQ ID No. 33), ADM-NH₂ (SEQ ID No. 20), ADM-Gly (SEQ ID No. 21) and CT-proADM (SEQ ID No. 34).

2. A method for (a) diagnosing or predicting the risk of life-threatening deterioration or an adverse event or (b) prognosing the severity or (c) predicting or monitoring the success of a therapy or intervention in a patient infected with a Corona virus according to embodiment 1, wherein said Corona Virus is selected from the group comprising Sars-CoV-1, Sars-CoV-2, MERS-CoV, in particular Sars-CoV-2.

3. A method for (a) diagnosing or predicting the risk of life-threatening deterioration or an adverse event or (b) prognosing the severity or (c) predicting or monitoring the success of a therapy or intervention in a patient infected with a Corona virus according to embodiment 1 or 2, wherein said adverse event is selected from the group comprising death, organ dysfunction, shock.

4. A method for (a) diagnosing or predicting the risk of life-threatening deterioration or an adverse event or (b) prognosing the severity or (c) predicting or monitoring the success of a therapy or intervention in a patient infected with a Corona virus according to embodiments 1 to 3, wherein said level of pro-Adrenomedullin or fragment thereof is above a pre-determined threshold.

5. A method for (a) diagnosing or predicting the risk of life-threatening deterioration or an adverse event or (b) prognosing the severity or (c) predicting or monitoring the success of a therapy or intervention in a patient infected with a Corona virus according to embodiments 1 to 4, wherein said fragment is MR-proADM (SEQ ID No. 33), and the predetermined threshold of MR-proADM in a sample of bodily fluid of said subject is between 0.5 and 2 nmol/L, preferably between 0.7 and 1.5 nmol/L, preferably between 0.8 and 1.2 nmol/L, most preferred a threshold of 1 nmol/L is applied.

6. A method for (a) diagnosing or predicting the risk of life-threatening deterioration or an adverse event or (b) prognosing the severity or (c) predicting or monitoring the success of a therapy or intervention in a patient infected with a Corona virus according to embodiments 1 to 4, wherein said fragment is ADM-NH₂ (SEQ ID No. 20), and the predetermined threshold of ADM-NH₂ (SEQ ID No. 20) in a sample of bodily fluid of said subject is between 40 and 100 pg/mL, more preferred between 50 and 90 pg/mL, even more preferred between 60 and 80 pg/mL, most preferred said threshold is 70 pg/mL.

7. A method for (a) diagnosing or predicting the risk of life-threatening deterioration or an adverse event or (b) prognosing the severity or (c) predicting or monitoring the success of a therapy or intervention in a patient infected with a Corona virus according to embodiments 1 to 6, wherein said patient has a SOFA score equal or greater than 3, preferably equal or greater than 7 or said patient has a quickSOFA score equal or greater than 1, preferably equal or greater than 2.

8. A method for (a) diagnosing or predicting the risk of life-threatening deterioration or an adverse event or (b) prognosing the severity or (c) predicting or monitoring the success of a therapy or intervention in a patient infected with a Corona virus according to embodiments 1 to 7, wherein said patient has a level of D-dimer equal or greater than 0.5 μg/ml, preferably equal or greater than 1.0 μg/ml.

9. A method for (a) diagnosing or predicting the risk of life-threatening deterioration or an adverse event or (b) prognosing the severity or (c) predicting or monitoring the success of a therapy or intervention in a patient infected with a Corona virus according to embodiments 1 to 8, wherein the level of pro-Adrenomedullin or fragment thereof is determined by contacting said sample of bodily fluid with a capture binder that binds specifically to pro-Adrenomedullin or fragment thereof.

10. A method for (a) diagnosing or predicting the risk of life-threatening deterioration or an adverse event or (b) prognosing the severity or (c) predicting or monitoring the success of a therapy or intervention in a patient infected with a Corona virus according to embodiments 1 to 9, wherein said determination comprises the use of a capture-binder that binds specifically to pro-Adrenomedullin or fragment thereof wherein said capture-binder may be selected from the group of antibody, antibody fragment or non-IgG scaffold.

11. A method for (a) diagnosing or predicting the risk of life-threatening deterioration or an adverse event or (b) prognosing the severity or (c) predicting or monitoring the success of a therapy or intervention in a patient infected with a Corona virus according to embodiments 1 to 10, wherein the level of pro-Adrenomedullin or fragment thereof is determined in a bodily fluid sample of said subject and wherein said determination comprises the use of a capture-binder that binds specifically to pro-Adrenomedullin or fragment thereof wherein said capture-binder is an antibody.

12. A method for (a) diagnosing or predicting the risk of life-threatening deterioration or an adverse event or (b) prognosing the severity or (c) predicting or monitoring the success of a therapy or intervention in a patient infected with a Corona virus according to embodiments 1 to 11, wherein the level of pro-Adrenomedullin or fragment thereof is determined in a bodily fluid sample of said subject and wherein said determination comprises the use of a capture-binder that binds specifically to level of pro-Adrenomedullin or fragment thereof, wherein said capture-binder is immobilized on a surface.

13. A method for (a) diagnosing or predicting the risk of life-threatening deterioration or an adverse event or (b) prognosing the severity or (c) predicting or monitoring the success of a therapy or intervention in a patient infected with a Corona virus according to embodiments 1 to 12, wherein said patient is treated with an Anti-adrenomedullin (ADM) antibody or anti-ADM antibody fragment or anti-ADM non-Ig scaffold wherein said anti-ADM antibody or anti-ADM fragment or anti-ADM non-Ig scaffold binds to the N-terminal and/or mid-regional part (aa 1-42) of ADM-Gly and/or ADM-NH₂:

(SEQ ID No. 23)
YRQSMNNFQGLRSFGCRFGTCTVQKLAHQIYQFTDKDKDNVA.

14. Anti-adrenomedullin (ADM) antibody or anti-ADM antibody fragment or anti-ADM non-Ig scaffold for use in therapy or intervention in a patient infected with a Corona virus.

15. Anti-adrenomedullin (ADM) antibody or anti-ADM antibody fragment or anti-ADM non-Ig scaffold for use in therapy or intervention in a patient infected with a Corona virus according to embodiment 14, wherein said Corona Virus is selected from the group comprising Sars-CoV-1, Sars-CoV-2, MERS-CoV, in particular Sars-CoV-2.

16. Anti-adrenomedullin (ADM) antibody or anti-ADM antibody fragment or anti-ADM non-Ig scaffold for use in therapy or intervention in a patient infected with a Corona virus according to embodiment 14 or 15, wherein said patient has a level of pro-Adrenomedullin or fragment thereof in a sample of bodily fluid of said subject that is above a predetermined threshold or higher than a previously measured level of pro-Adrenomedullin when determined by a method according to any of claims 1-12.

17. Anti-adrenomedullin (ADM) antibody or anti-ADM antibody fragment or anti-ADM non-Ig scaffold for use in therapy or intervention in a patient infected with a Corona virus according to embodiments 14 to 16, wherein said patient has a SOFA score equal or greater than 3, preferably equal or greater than 7 or said patient has a quickSOFA score equal or greater than 1, preferably equal or greater than 2.

18. Anti-adrenomedullin (ADM) antibody or anti-ADM antibody fragment or anti-ADM non-Ig scaffold for use in therapy or intervention in a patient infected with a Corona virus according to embodiments 14 to 17, wherein said patient has a level of D-dimer equal or greater than 0.5 μg/ml, preferably equal or greater than 1.0 μg/ml.

19. Anti-adrenomedullin (ADM) antibody or anti-ADM antibody fragment or anti-ADM non-Ig scaffold for use in therapy or intervention in a patient infected with a Corona virus according to embodiments 14 to 18, wherein said anti-ADM antibody or anti-ADM fragment or anti-ADM non-Ig scaffold binds to the N-terminal (amino acid 1-21) of ADM-Gly and/or ADM-NH₂:

(SEQ ID No. 14)
YRQSMNNFQGLRSFGCRFGTC.

20. Anti-adrenomedullin (ADM) antibody or anti-ADM antibody fragment or anti-ADM non-Ig scaffold for use in therapy or intervention in a patient infected with a Corona virus according to embodiments 14-19, wherein said Anti-adrenomedullin (ADM) antibody or anti-ADM antibody fragment or anti-ADM non-Ig scaffold exhibits a minimum binding affinity to pro-Adrenomedullin or a fragment thereof of equal or less than 10-7 M.

21. Anti-adrenomedullin (ADM) antibody or anti-ADM antibody fragment or anti-ADM non-Ig scaffold for use in therapy or intervention in a patient infected with a Corona virus according to embodiments 14-20, wherein said Anti-adrenomedullin (ADM) antibody or anti-ADM antibody fragment or anti-ADM non-Ig scaffold wherein said antibody or fragment or scaffold blocks the bioactivity of ADM not more than 80%, preferably not more than 50%.

22. Anti-adrenomedullin (ADM) antibody or anti-ADM antibody fragment or anti-ADM non-Ig scaffold for use in therapy or intervention in a patient infected with a Corona virus according to embodiments 14-21, wherein said antibody is a monoclonal antibody or monoclonal antibody fragment.

23. Anti-adrenomedullin (ADM) antibody or anti-ADM antibody fragment or anti-ADM non-Ig scaffold for use in therapy or intervention in a patient infected with a Corona virus according to embodiment 22, wherein the complementarity determining regions (CDR's) in the heavy chain comprises the sequences:

CDR1:
SEQ ID NO: 1
GYTFSRYW
CDR2:
SEQ ID NO: 2
ILPGSGST
CDR3:
SEQ ID NO: 3
TEGYEYDGFDY

and the complementarity determining regions (CDR's) in the light chain comprises the sequences:

CDR1:
SEQ ID NO: 4
QSIVYSNGNTY
CDR2:
RVS
CDR3:
SEQ ID NO: 5
FQGSHIPYT

24. Anti-adrenomedullin (ADM) antibody or anti-ADM antibody fragment or anti-ADM non-Ig scaffold for use in therapy or intervention in a patient infected with a Corona virus according to embodiment 23, wherein said antibody or fragment comprises a sequence selected from the group comprising as a VH region:

(AM-VH-C)
SEQ ID NO: 6
QVQLQQSGAELMKPGASVKISCKATGYTFSRYWIEWVKQRPGHGLEWIGE
ILPGSGSTNYNEKFKGKATITADTSSNTAYMQLSSLTSEDSAVYYCTEGY
EYDGFDYWGQGTTLTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPK
(AM-VH1)
SEQ ID NO: 7
QVQLVQSGAEVKKPGSSVKVSCKASGYTFSRYWISWVRQAPGQGLEWMGR
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPK
(AM-VH2-E40)
SEQ ID NO: 8
QVQLVQSGAEVKKPGSSVKVSCKASGYTFSRYWIEWVRQAPGQGLEWMGR
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPK
(AM-VH3-T26-E55)
SEQ ID NO: 9
QVQLVQSGAEVKKPGSSVKVSCKATGYTFSRYWISWVRQAPGQGLEWMGE
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPK
(AM-VH4-T26-E40-E55)
SEQ ID NO: 10
QVQLVQSGAEVKKPGSSVKVSCKATGYTFSRYWIEWVRQAPGQGLEWMGE
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNRKPSNTKVDKRVEPK

or a sequence that is >80% identical to each of the above depicted sequences respectively, and comprises a sequence selected from the group comprising the following sequence as a VL region:

(AM-VL-C)
SEQ ID NO: 11
DVLLSQTPLSLPVSLGDQATISCRSSQSIVYSNGNTYLEWYLQKPGQSPK
LLIYRVSNRFSGVPDRFSGSGSGTDFTLKISRVEAEDLGVYYCFQGSHIP
YTFGGGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
(AM-VL1)
SEQ ID NO: 12
DVVMTQSPLSLPVTLGQPASISCRSSQSIVYSNGNTYLNWFQQRPGQSPR
RLIYRVSNRDSGVPDRFSGSGSGTDFTLKISRVEAEDVGVYYCFQGSHIP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
(AM-VL2-E40)
SEQ ID NO: 13
DVVMTQSPLSLPVTLGQPASISCRSSQSIVYSNGNTYLEWFQQRPGQSPR
RLIYRVSNRDSGVPDRFSGSGSGTDFTLKISRVEAEDVGVYYCFQGSHIP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

or a sequence that is >80% identical to each of the above depicted sequences.

25. Adrenomedullin (ADM) antibody or anti-ADM antibody fragment or anti-ADM non-Ig scaffold for use in therapy or intervention in a patient infected with a Corona virus according to any of embodiments 23 to 24, wherein said antibody or fragment comprises the following sequence as a heavy chain:

SEQ ID NO: 35
QVQLVQSGAEVKKPGSSVKVSCKASGYTFSRYWIEWVRQAPGQGLEWIGE
ILPGSGSTNYNQKFQGRVTITADTSTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK

or a sequence that is >95% identical to it,

and comprises the following sequence as a light chain:

SEQ ID NO: 36
DVVLTQSPLSLPVTLGQPASISCRSSQSIVYSNGNTYLEWYLQRPGQSPR
LLIYRVSNRFSGVPDRFSGSGSGTDFTLKISRVEAEDVGVYYCFQGSHIP
YTFGGGTKLEIKRTVAAPSVFIFPPSDEQLKSGTASVVCLLNNFYPREAK
VQWKVDNALQSGNSQESVTEQDSKDSTYSLSSTLTLSKADYEKEIKVYAC
EVTHQGLSSPVTKSFNRGEC

or a sequence that is >95% identical to it.

26. Anti-adrenomedullin (ADM) antibody or anti-ADM antibody fragment or anti-ADM non-Ig scaffold for use in therapy or intervention in a patient infected with a Corona virus according to any of embodiments 23 to 25, wherein said monoclonal antibody or antibody fragment is a humanized monoclonal antibody or humanized monoclonal antibody fragment.

27. Anti-adrenomedullin (ADM) antibody or anti-ADM antibody fragment or anti-ADM non-Ig scaffold for use in therapy or intervention in a patient infected with a Corona virus according to embodiments 14-26, wherein said Anti-adrenomedullin (ADM) antibody or anti-ADM antibody fragment or anti-ADM non-Ig scaffold is an monoclonal antibody and is Adrecizumab and comprises the following sequence as a heavy chain:

SEQ ID NO: 35
QVQLVQSGAEVKKPGSSVKVSCKASGYTFSRYWIEWVRQAPQGLEWIGEI
LPGSGSTNYNQKFQGRVTITADTSTSTAYMELSSLRSEDTAVYYCTEGYE
YDGFDYWGQGTTVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK

and comprises the following sequence as a light chain:

SEQ ID NO: 36
DVVLTQSPLSLPVTLGQPASISCRSSQSIVYSNGNTYLEWYLQRPGQSPR
LLIYRVSNRFSGVPDRFSGSGSGTDFTLKISRVEAEDVGVYYCFQGSHIP
YTFGGGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

or a biosimilar thereof.

28. Anti-adrenomedullin (ADM) antibody or anti-ADM antibody fragment or anti-ADM non-Ig scaffold for use in therapy or intervention in a patient with compromised lung function and/or acute respiratory distress syndrome (ARDS).

29. Anti-adrenomedullin (ADM) antibody or anti-ADM antibody fragment or anti-ADM non-Ig scaffold for use in therapy or intervention in a patient with compromised lung function and/or acute respiratory distress syndrome (ARDS) according to embodiment 28, wherein said patient has a Horowitz index below 300, in particular below 200, in particular below 100 and/or said patient is in need of mechanical ventilation.

30. Anti-adrenomedullin (ADM) antibody or anti-ADM antibody fragment or anti-ADM non-Ig scaffold for use in therapy or intervention in a patient with compromised lung function and/or acute respiratory distress syndrome (ARDS) according to embodiment 28 or 29, wherein said patient has a level of pro-Adrenomedullin or fragment thereof in a sample of bodily fluid of said subject that is above a predetermined threshold or higher than a previously measured level of pro-Adrenomedullin when determined by a method according to any of claims 1-12.

31. Anti-adrenomedullin (ADM) antibody or anti-ADM antibody fragment or anti-ADM non-Ig scaffold for use in therapy or intervention in a patient with compromised lung function and/or acute respiratory distress syndrome (ARDS) according to embodiments 28 to 30, wherein said patient has a SOFA score equal or greater than 3, preferably equal or greater than 7 or said patient has a quickSOFA score equal or greater than 1, preferably equal or greater than 2.

32. Anti-adrenomedullin (ADM) antibody or anti-ADM antibody fragment or anti-ADM non-Ig scaffold for use in therapy or intervention in a patient with compromised lung function and/or acute respiratory distress syndrome (ARDS) according to embodiments 28 to 31, wherein said patient has a level of D-dimer equal or greater than 0.5 μg/ml, preferably equal or greater than 1.0 μg/ml.

33. Anti-adrenomedullin (ADM) antibody or anti-ADM antibody fragment or anti-ADM non-Ig scaffold for use in therapy or intervention i in a patient with compromised lung function and/or acute respiratory distress syndrome (ARDS) according to embodiments 28 to 32, wherein said anti-ADM antibody or anti-ADM fragment or anti-ADM non-Ig scaffold binds to the N-terminal (amino acid 1-21) of ADM-Gly and/or ADM-NH2: YRQSMNNFQGLRSFGCRFGTC (SEQ ID No. 14).

34. Anti-adrenomedullin (ADM) antibody or anti-ADM antibody fragment or anti-ADM non-Ig scaffold for use in therapy or intervention in a patient with compromised lung function and/or acute respiratory distress syndrome (ARDS) according to embodiments 28-33, wherein said Anti-adrenomedullin (ADM) antibody or anti-ADM antibody fragment or anti-ADM non-Ig scaffold exhibits a minimum binding affinity to pro-Adrenomedullin or a fragment thereof of equal or less than 10-7 M.

35. Anti-adrenomedullin (ADM) antibody or anti-ADM antibody fragment or anti-ADM non-Ig scaffold for use in therapy or intervention in a patient with compromised lung function and/or acute respiratory distress syndrome (ARDS) according to embodiments 28-34, wherein said Anti-adrenomedullin (ADM) antibody or anti-ADM antibody fragment or anti-ADM non-Ig scaffold wherein said antibody or fragment or scaffold blocks the bioactivity of ADM not more than 80%, preferably not more than 50%.

36. Anti-adrenomedullin (ADM) antibody or anti-ADM antibody fragment or anti-ADM non-Ig scaffold for use in therapy or intervention in a patient with compromised lung function and/or acute respiratory distress syndrome (ARDS) according to embodiments 28-35, wherein said antibody is a monoclonal antibody or monoclonal antibody fragment.

37. Anti-adrenomedullin (ADM) antibody or anti-ADM antibody fragment or anti-ADM non-Ig scaffold for use in therapy or intervention in a patient with compromised lung function and/or acute respiratory distress syndrome (ARDS) according to embodiment 36, wherein the complementarity determining regions (CDR's) in the heavy chain comprises the sequences:

CDR1:
SEQ ID NO: 1
GYTFSRYW
CDR2:
SEQ ID NO: 2
ILPGSGST
CDR3:
SEQ ID NO: 3
TEGYEYDGFDY

and the complementarity determining regions (CDR's) in the light chain comprises the sequences:

CDR1:
SEQ ID NO: 4
QSIVYSNGNTY
CDR2:
RVS
CDR3:
SEQ ID NO: 5
FQGSHIPYT

38. Anti-adrenomedullin (ADM) antibody or anti-ADM antibody fragment or anti-ADM non-Ig scaffold for use in therapy or intervention in a patient with compromised lung function and/or acute respiratory distress syndrome (ARDS) according to embodiment 37, wherein said antibody or fragment comprises a sequence selected from the group comprising as a VH region:

(AM-VH-C)
SEQ ID NO: 6
QVQLQQSGAELMKPGASVKISCKATGYTFSRYWIEWVKQRPGHGLEWIGE
ILPGSGSTNYNEKFKGKATITADTSSNTAYMQLSSLTSEDSAVYYCTEGY
EYDGFDYWGQGTTLTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPK
(AM-VH1)
SEQ ID NO: 7
QVQLVQSGAEVKKPGSSVKVSCKASGYTFSRYWISWVRQAPGQGLEWMGR
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPK
(AM-VH2-E40)
SEQ ID NO: 8
QVQLVQSGAEVKKPGSSVKVSCKASGYTFSRYWIEWVRQAPGQGLEWMGR
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPK
(AM-VH3-T26-E55)
SEQ ID NO: 9
QVQLVQSGAEVKKPGSSVKVSCKATGYTFSRYWISWVRQAPGQGLEWMGE
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPK
(AM-VH4-T26-E40-E55)
SEQ ID NO: 10
QVQLVQSGAEVKKPGSSVKVSCKATGYTFSRYWIEWVRQAPGQGLEWMGE
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPK

or a sequence that is >80% identical to each of the above depicted sequences respectively, and comprises a sequence selected from the group comprising the following sequence as a VL region:

(AM-VL-C)
SEQ ID NO: 11
DVLLSQTPLSLPVSLGDQATISCRSSQSIVYSNGNTYLEWYLQKPGQSPK
LLIYRVSNRFSGVPDRFSGSGSGTDFTLKISRVEAEDLGVYYCFQGSHIP
YTFGGGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
(AM-VL1)
SEQ ID NO: 12
DVVMTQSPLSLPVTLGQPASISCRSSQSIVYSNGNTYLNWFQQRPGQSPR
RLIYRVSNRDSGVPDRFSGSGSGTDFTLKISRVEAEDVGVYYCFQGSHIP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
(AM-VL2-E40)
SEQ ID NO: 13
DVVMTQSPLSLPVTLGQPASISCRSSQSIVYSNGNTYLEWFQQRPGQSPR
RLIYRVSNRDSGVPDRFSGSGSGTDFTLKISRVEAEDVGVYYCFQGSHIP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

or a sequence that is >80% identical to each of the above depicted sequences.

39. Adrenomedullin (ADM) antibody or anti-ADM antibody fragment or anti-ADM non-Ig scaffold for use in therapy or intervention in a patient with compromised lung function and/or acute respiratory distress syndrome (ARDS) according to any of embodiments 37 to 38, wherein said antibody or fragment comprises the following sequence as a heavy chain:

SEQ ID NO: 35
QVQLVQSGAEVKKPGSSVKVSCKASGYTFSRYWIEWVRQAPGQGLEWIGE
ILPGSGSTNYNQKFQGRVTITADTSTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK

or a sequence that is >95% identical to it,

and comprises the following sequence as a light chain:

SEQ ID NO: 36
DVVLTQSPLSLPVTLGQPASISCRSSQSIVYSNGNTYLEWYLQRPGQSPR
LLIYRVSNRFSGVPDRFSGSGSGTDFTLKISRVEAEDVGVYYCFQGSHIP
YTFGGGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

or a sequence that is >95% identical to it.

40. Anti-adrenomedullin (ADM) antibody or anti-ADM antibody fragment or anti-ADM non-Ig scaffold for use in therapy or intervention in a patient with compromised lung function and/or acute respiratory distress syndrome (ARDS) according to any of embodiments 37 to 39, wherein said monoclonal antibody or antibody fragment is a humanized monoclonal antibody or humanized monoclonal antibody fragment.

41. Anti-adrenomedullin (ADM) antibody or anti-ADM antibody fragment or anti-ADM non-Ig scaffold for use in therapy or intervention in a patient with compromised lung function and/or acute respiratory distress syndrome (ARDS) according to embodiments 28-40, wherein said Anti-adrenomedullin (ADM) antibody or anti-ADM antibody fragment or anti-ADM non-Ig scaffold is an monoclonal antibody and is Adrecizumab and comprises the following sequence as a heavy chain:

SEQ ID NO: 35
QVQLVQSGAEVKKPGSSVKVSCKASGYTFSRYWIEWVRQAPGQGLEWIGE
ILPGSGSTNYNQKFQGRVTITADTSTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK

and comprises the following sequence as a light chain:

SEQ ID NO: 36
DVVLTQSPLSLPVTLGQPASISCRSSQSIVYSNGNTYLEWYLQRPGQSPR
LLIYRVSNRFSGVPDRFSGSGSGTDFTLKISRVEAEDVGVYYCFQGSHIP
YTFGGGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

or a biosimilar thereof.

### EXAMPLES

It should be emphasized that the antibodies, antibody fragments and non-Ig scaffolds of the example portion in accordance with the invention are binding to ADM, and thus should be considered as anti-ADM antibodies/antibody fragments/non-Ig scaffolds.

### Example 1—Generation of Antibodies and Determination of their Affinity Constants

Several anti-human and anti-murine ADM antibodies were produced and their affinity constants were determined (see tables 2 and 3).

Peptides/Conjugates for Immunization:

Peptides for immunization were synthesized, see Table 2, (JPT Technologies, Berlin, Germany) with an additional N-terminal Cystein (if no Cystein is present within the selected ADM-sequence) residue for conjugation of the peptides to Bovine Serum Albumin (BSA). The peptides were covalently linked to BSA by using Sulfolink-coupling gel (Perbio-science, Bonn, Germany). The coupling procedure was performed according to the manual of Perbio.

Mouse Monoclonal Antibody Production:

A Balb/c mouse was immunized with 100 μg Peptide-BSA-Conjugate at day 0 and 14 (emulsified in 104 μl complete Freund's adjuvant) and 50 μs at day 21 and 28 (in 104 μl incomplete Freund's adjuvant). Three days before the fusion experiment was performed, the animal received 50 μg of the conjugate dissolved in 100 μl saline, given as one intraperitoneal and one intra-venous injection. Splenocytes from the immunized mouse and cells of the myeloma cell line SP2/0 were fused with 1 ml 50% polyethylene glycol for 30 s at 37° C. After washing, the cells were seeded in 96-well cell culture plates. Hybrid clones were selected by growing in HAT medium [RPMI 1640 culture medium supplemented with 20% fetal calf serum and HAT-Supplement]. After two weeks the HAT medium is replaced with HT Medium for three passages followed by returning to the normal cell culture medium.

The cell culture supernatants were primary screened for antigen specific IgG antibodies three weeks after fusion. The positive tested microcultures were transferred into 24-well plates for propagation. After retesting, the selected cultures were cloned and re-cloned using the limiting-dilution technique and the isotypes were determined (see also Lane, R. D. 1985. J. Immunol. Meth. 81: 223-228; Ziegler et al. 1996. Horm. Metab. Res. 28: 11-15).

Antibodies were produced via standard antibody production methods (Marx et al, 1997. Monoclonal Antibody Production, ATLA 25, 121) and purified via Protein A. The antibody purities were >95% based on SDS gel electrophoresis analysis.

Human Antibody Production by Means of Phage Display:

The human naive antibody gene libraries HALT/8 were used for the isolation of recombinant single chain F-Variable domains (scFv) against adrenomedullin peptide. The antibody gene libraries were screened with a panning strategy comprising the use of peptides containing a biotin tag linked via two different spacers to the adrenomedullin peptide sequence. A mix of panning rounds using non-specifically bound antigen and streptavidin bound antigen were used to minimize background of non-specific binders. The eluted phages from the third round of panning have been used for the generation of monoclonal scFv expressing E. coli strains. Supernatant from the cultivation of these clonal strains has been directly used for an antigen ELISA testing (see also Hust et al. 2011. Journal of Biotechnology 152, 159-170; Schutte et al. 2009. PLoS One 4, e6625).

Positive clones have been selected based on positive ELISA signal for antigen and negative for streptavidin coated micro titer plates. For further characterizations the scFv open reading frame has been cloned into the expression plasmid pOPE107 (Hust et al., J. Biotechn. 2011), captured from the culture supernatant via immobilized metal ion affinity chromatography and purified by a size exclusion chromatography.

Affinity Constants:

To determine the affinity of the antibodies to Adrenomedullin, the kinetics of binding of Adrenomedullin to immobilized antibody was determined by means of label-free surface plasmon resonance using a Biacore 2000 system (GE Healthcare Europe GmbH, Freiburg, Germany).

Reversible immobilization of the antibodies was performed using an anti-mouse Fc antibody covalently coupled in high density to a CM5 sensor surface according to the manufacturer's instructions (mouse antibody capture kit; GE Healthcare). (Lorenz et al. 2011. Antimicrob Agents Chemother. 55(1): 165-173).

The monoclonal antibodies were raised against the below depicted ADM regions of human and murine ADM, respectively. The following table represents a selection of obtained antibodies used in further experiments. Selection was based on target region:

TABLE 2

| | | | | Affinity |
|---|---|---|---|---|
| Sequence | Antigen/ | ADM | Desig- | constants |
| Number | Immunogen | Region | nation | Kd (M) |
| SEQ ID: 14 | YRQSMNNFQ | 1-21 | NT-H | 5.9 × 10⁻⁹ |
| | GLRSFGCRF | | | |
| | GTC | | | |
| SEQ ID: 15 | CTVQKLAHQ | 21-32 | MR-H | 2 × 10⁻⁹ |
| | IYQ | | | |
| SEQ ID: 16 | CAPRSKISP | C-42-52 | CT-H | 1.1 × 10⁻⁹ |
| | QGY-NH2 | | | |
| SEQ ID: 17 | YRQSMNQGSR | 1-19 | NT-M | 3.9 × 10⁻⁹ |
| | SNGCRFGTC | | | |
| SEQ ID: 18 | CTFQKLAHQ | 19-31 | MR-M | 4.5 × 10⁻¹⁰ |
| | IYQ | | | |
| SEQ ID: 19 | CAPRNKISPQ | C-40-50 | CT-M | 9 × 10⁻⁹ |
| | GY-NH2 | | | |

The following is a list of further obtained monoclonal antibodies:

TABLE 3

| | | | | Max. |
|---|---|---|---|---|
| | | | | inhibition |
| | | Clone | | bioassay (%) |
| Target | Source | number | Affinity (M) | (see example 2) |
| NT-M | Mouse | ADM/63 | 5.8 × 10⁻⁹ | 45 |
| NT-M | Mouse | ADM/364 | 2.2 × 10⁻⁸ | 48 |
| NT-M | Mouse | ADM/365 | 3.0 × 10⁻⁸ | |
| NT-M | Mouse | ADM/366 | 1.7 × 10⁻⁸ | |
| NT-M | Mouse | ADM/367 | 1.3 × 10⁻⁸ | |
| NT-M | Mouse | ADM/368 | 1.9 × 10⁻⁸ | |
| NT-M | Mouse | ADM/369 | 2.0 × 10⁻⁸ | |
| NT-M | Mouse | ADM/370 | 1.6 × 10⁻⁸ | |
| NT-M | Mouse | ADM/371 | 2.0 × 10⁻⁸ | |
| NT-M | Mouse | ADM/372 | 2.5 × 10⁻⁸ | |
| NT-M | Mouse | ADM/373 | 1.8 × 10⁻⁸ | |
| NT-M | Mouse | ADM/377 | 1.5 × 10⁻⁸ | |
| NT-M | Mouse | ADM/378 | 2.2 × 10⁻⁸ | |
| NT-M | Mouse | ADM/379 | 1.6 × 10⁻⁸ | |
| NT-M | Mouse | ADM/380 | 1.8 × 10⁻⁸ | |
| NT-M | Mouse | ADM/381 | 2.4 × 10⁻⁸ | |
| NT-M | Mouse | ADM/382 | 1.6 × 10⁻⁸ | |
| NT-M | Mouse | ADM/383 | 1.8 × 10⁻⁸ | |
| NT-M | Mouse | ADM/384 | 1.7 × 10⁻⁸ | |
| NT-M | Mouse | ADM/385 | 1.7 × 10⁻⁸ | |
| NT-M | Mouse | ADM/403 | 1.2 × 10⁻⁸ | |
| NT-M | Mouse | ADM/395 | 1.2 × 10⁻⁸ | |
| NT-M | Mouse | ADM/396 | 3.0 × 10⁻⁸ | |
| NT-M | Mouse | ADM/397 | 1.5 × 10⁻⁸ | |
| MR-M | Mouse | ADM/38 | 4.5 × 10⁻¹⁰ | 68 |
| MR-M | Mouse | ADM/39 | 5.9 × 10⁻⁹ | 72 |
| CT-M | Mouse | ADM/65 | 9.0 × 10⁻⁹ | 100 |
| CT-M | Mouse | ADM/66 | 1.6 × 10⁻⁸ | 100 |
| NT-H | Mouse | ADM/33 | 5.9 × 10⁻⁸ | 38 |
| NT-H | Mouse | ADM/34 | 1.6 × 10⁻⁸ | 22 |
| MR-H | Mouse | ADM/41 | 1.2 × 10⁻⁸ | 67 |
| MR-H | Mouse | ADM/42 | <1 × 10⁻⁸ | |
| MR-H | Mouse | ADM/43 | 2.0 × 10⁻⁹ | 73 |
| MR-H | Mouse | ADM/44 | <1 × 10⁻⁸ | |
| CT-H | Mouse | ADM/15 | <1 × 10⁻⁸ | |
| CT-H | Mouse | ADM/16 | 1.1 × 10⁻⁹ | 100 |
| CT-H | Mouse | ADM/17 | 3.7 × 10⁻⁹ | 100 |
| CT-H | Mouse | ADM/18 | <1 × 10⁻⁸ | |
| hADM | Phage display | ADM/A7 | <1 × 10⁻⁸ | |
| hADM | Phage display | ADM/B7 | <1 × 10⁻⁸ | |
| hADM | Phage display | ADM/C7 | <1 × 10⁻⁸ | |
| hADM | Phage display | ADM/G3 | <1 × 10⁻⁸ | |
| hADM | Phage display | ADM/B6 | <1 × 10⁻⁸ | |
| hADM | Phage display | ADM/B11 | <1 × 10⁻⁸ | |
| hADM | Phage display | ADM/D8 | <1 × 10⁻⁸ | |
| hADM | Phage display | ADM/D11 | <1 × 10⁻⁸ | |
| hADM | Phage display | ADM/G12 | <1 × 10⁻⁸ | |

Generation of Antibody Fragments by Enzymatic Digestion:

The generation of Fab and F(ab)₂ fragments was done by enzymatic digestion of the murine full-length antibody NT-M. Antibody NT-M was digested using a) the pepsin-based F(ab)₂ Preparation Kit (Pierce 44988) and b) the papain-based Fab Preparation Kit (Pierce 44985). The fragmentation procedures were performed according to the instructions provided by the supplier. Digestion was carried out in case of F(ab)₂-fragmentation for 8 h at 37° C. The Fab-fragmentation digestion was carried out for 16 h, respectively.

Procedure for Fab Generation and Purification:

The immobilized papain was equilibrated by washing the resin with 0.5 ml of digestion buffer and centrifuging the column at 5000×g for 1 minute. The buffer was discarded afterwards. The desalting column was prepared by removing the storage solution and washing it with digestion buffer, centrifuging it each time afterwards at 1000×g for 2 minutes. 0.5 ml of the prepared IgG sample where added to the spin column tube containing the equilibrated immobilized Papain. Incubation time of the digestion reaction was done for 16 h on a tabletop rocker at 37° C. The column was centrifuged at 5000×g for 1 minute to separate digest from the immobilized Papain. Afterwards the resin was washed with 0.5 ml PBS and centrifuged at 5000×g for 1 minute. The wash fraction was added to the digested antibody that the total sample volume was 1.0 ml. The NAb Protein A Column was equilibrated with PBS and IgG elution buffer at room temperature. The column was centrifuged for 1 minute to remove storage solution (contains 0.02% sodium azide) and equilibrated by adding 2 ml of PBS, centrifuge again for 1 minute and the flow-through discarded. The sample was applied to the column and resuspended by inversion. Incubation was done at room temperature with end-over-end mixing for 10 minutes. The column was centrifuged for 1 minute, saving the flow-through with the Fab fragments. (References: Coulter and Harris 1983.1 Immunol. Meth. 59, 199-203; Lindner et al. 2010. Cancer Res. 70, 277-87; Kaufmann et al. 2010. PNAS. 107, 18950-5; Chen et al. 2010. PNAS. 107, 14727-32; Uysal et al. 20091 Exp. Med 206, 449-62; Thomas et al. 2009. J. Exp. Med. 206, 1913-27; Kong et al. 2009 J. Cell Biol. 185, 1275-840).

Procedure for Generation and Purification of F(Ab′)₂ Fragments:

The immobilized Pepsin was equilibrated by washing the resin with 0.5 ml of digestion buffer and centrifuging the column at 5000×g for 1 minute. The buffer was discarded afterwards. The desalting column was prepared by removing the storage solution and washing it with digestion buffer, centrifuging it each time afterwards at 1000×g for 2 minutes. 0.5 ml of the prepared IgG sample where added to the spin column tube containing the equilibrated immobilized Pepsin. Incubation time of the digestion reaction was done for 16 h on a tabletop rocker at 37° C. The column was centrifuged at 5000×g for 1 minute to separate digest from the immobilized Papain. Afterwards the resin was washed with 0.5 mL PBS and centrifuged at 5000×g for 1 minute. The wash fraction was added to the digested antibody that the total sample volume was 1.0 ml. The NAb Protein A Column was equilibrated with PBS and IgG Elution Buffer at room temperature. The column was centrifuged for 1 minute to remove storage solution (contains 0.02% sodium azide) and equilibrated by adding 2 mL of PBS, centrifuge again for 1 minute and the flow-through discarded. The sample was applied to the column and resuspended by inversion. Incubation was done at room temperature with end-over-end mixing for 10 minutes. The column was centrifuged for 1 minute, saving the flow-through with the Fab fragments. (References: Mariani et al. 1991. Mol. Immunol. 28: 69-77; Beale 1987. Exp Comp Immunol 11:287-96; Ellerson et al. 1972. FEBS Letters 24(3):318-22; Kerbel and Elliot 1983. Meth Enzymol 93:113-147; Kulkarni et al. 1985. Cancer Immunol Immunotherapy 19:211-4; Lamoyi 1986. Meth Enzymol 121:652-663; Parham et al. 1982. J Immunol Meth 53:133-73; Raychaudhuri et al. 1985. Mol Immunol 22(9):1009-19; Rousseaux et al. 1980. Mol Immunol 17:469-82; Rousseaux et al. 1983. J Immunol Meth 64:141-6; Wilson et al. 1991. J Immunol Meth 138:111-9).

NT-H-Antibody Fragment Humanization:

The antibody fragment was humanized by the CDR-grafting method (Jones et al. 1986. Nature 321, 522-525). The following steps were done to achieve the humanized sequence:

Total RNA was extracted from NT-H hybridomas using the Qiagen kit. For first-round RT-PCR the QIAGEN® OneStep RT-PCR Kit (Cat No. 210210) was used. RT-PCR was performed with primer sets specific for the heavy and light chains. For each RNA sample, 12 individual heavy chain and 11 light chain RT-PCR reactions were set up using degenerate forward primer mixtures covering the leader sequences of variable regions. Reverse primers are located in the constant regions of heavy and light chains. No restriction sites were engineered into the primers.

The reaction set up was as follows: 5× QIAGEN OneStep RT-PCR Buffer 5.0 dNTP Mix (containing 10 mM of each dNTP) 0.8 μl, Primer set 0.5 μl, QIAGEN® OneStep RT-PCR Enzyme Mix 0.8 μl, Template RNA 2.0 μl, RNase-free water to 20.0 μl, Total volume 20.0 μl PCR condition: Reverse transcription: 50° C., 30 min; Initial PCR activation: 95° C., 15 min Cycling: 20 cycles of 94° C., 25 sec; 54° C., 30 sec; 72° C., 30 sec; Final extension: 72° C., 10 min Second-round semi-nested PCR: The RT-PCR products from the first-round reactions were further amplified in the second-round PCR. 12 individual heavy chain and 11 light chain RT-PCR reactions were set up using semi-nested primer sets specific for antibody variable regions.

The reaction setup was as follows: 2×PCR mix 10 μl; Primer set 2 μl; First-round PCR product 8 μl; Total volume 20 μl; Hybridoma Antibody Cloning Report PCR condition: Initial denaturing of 5 min at 95° C.; 25 cycles of 95° C. for 25 sec, 57° C. for 30 sec, 68° C. for 30 sec; Final extension is 10 min 68° C.

After PCR is finished, run PCR reaction samples onto agarose gel to visualize DNA fragments amplified. After sequencing more than 15 cloned DNA fragments amplified by nested RT-PCR, several mouse antibody heavy and light chains have been cloned and appear correct. Protein sequence alignment and CDR analysis identifies one heavy chain and one light chain. After alignment with homologous human framework sequences, the resulting humanized sequence for the variable heavy chain is the following: see FIG. 5. As the amino acids on positions 26, 40 and 55 in the variable heavy chain and amino acid on position 40 in the variable light are critical to the binding properties, they may be reverted to the murine original. The resulting candidates are depicted below. (Padlan 1991. Mol. Immunol. 28, 489-498; Harris and Bajorath. 1995. Protein Sci. 4, 306-310).

Annotation for the antibody fragment sequences (SEQ ID No.: 7-13, 35 and 36): bold and underline are the CDR 1, 2, 3 chronologically arranged.

(AM-VH-C)
SEQ ID No.: 6
QVQLQQSGAELMKPGASVKISCKATGYTFSRYWIEWVKQRPGHGLEWIGE
ILPGSGSTNYNEKFKGKATITADTSSNTAYMQLSSLTSEDSAVYYCTEGY
EYDGFDYWGQGTTLTVSSASTKGPSVFPLAPSSKSTSGGTAALGCLVKDY
FPEPVTVSWNSGALTSGVHTFPAVLQSSGLYSLSSVVTVPSSSLGTQTYI
CNVNEEKPSNTKVDKRVEPK
(AM-VH1)
SEQ ID No.: 7
QVQLVQSGAEVKKPGSSVKVSCKASGYTFSRYWISWVRQAPGQGLEWMGR
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNEEKPSNTKVDKRVEPK
(AM-VH2-E40)
SEQ ID No.: 8
QVQLVQSGAEVKKPGSSVKVSCKASGYTFSRYWIEWVRQAPGQGLEWMGR
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNEEKPSNTKVDKRVEPK
(AM-VH3-T26-E55)
SEQ ID No.: 9
QVQLVQSGAEVKKPGSSVKVSCKATGYTFSRYWISWVRQAPGQGLEWMGE
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNEEKPSNTKVDKRVEPK
(AM-VH4-T26-E40-E55)
SEQ ID No.: 10
QVQLVQSGAEVKKPGSSVKVSCKATGYTFSRYWIEWVRQAPGQGLEWMGE
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNEEKPSNTKVDKRVEPK
(AM-VL-C)
SEQ ID No.: 11
DVLLSQTPLSLPVSLGDQATISCRSSQSIVYSNGNTYLEWYLQKPGQSPK
LLIYRVSNRFSGVPDRFSGSGSGTDFTLKISRVEAEDLGVYYCFQGSHIP
YTFGGGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
(AM-VL1)
SEQ ID No.: 12
DVVMTQSPLSLPVTLGQPASISCRSSQSIVYSNGNTYLNWFQQRPGQSPR
RLIYRVSNRDSGVPDRFSGSGSGTDFTLKISRVEAEDVGVYYCFQGSHIP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
(AM-VL2-E40)
SEQ ID No.: 13
DVVMTQSPLSLPVTLGQPASISCRSSQSIVYSNGNTYLEWFQQRPGQSPR
RLIYRVSNRDSGVPDRFSGSGSGTDFTLKISRVEAEDVGVYYCFQGSHIP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
SEQ ID No.: 35
QVQLVQSGAEVKKPGSSVKVSCKASGYTFSRYWIEWVRQAPGQGLEWIGE
ILPGSGSTNYNQKFQGRVTITADTSTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNEEKPSNTKVDKKVEPKSCDKTHTCPPCPAPELLGGPSVFLFPPKPK
DTLMISRTPEVTCVVVDVSEEEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
SEQ ID No.: 36
DVVLTQSPLSLPVTLGQPASISCRSSQSIVYSNGNTYLEWYLQRPGQSPR
LLIYRVSNRFSGVPDRFSGSGSGTDFTLKISRVEAEDVGVYYCFQGSHIP
YTFGGGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

### Example 2—Effect of Selected Anti-ADM-Antibodies on Anti-ADM-Bioactivity

The effect of selected ADM-antibodies on ADM-bioactivity was tested in a human recombinant Adrenomedullin receptor cAMP functional assay (Adrenomedullin Bioassay). The following materials were used: Cell line CHO-K1, Adrenomedullin receptor (CRLR+RAMP3), Receptor Accession Number Cell line (CRLR: U17473; RAMP3: AJ001016). CHO-K1 cells expressing human recombinant adrenomedullin receptor (FAST-027C) grown prior to the test in media is without antibiotic were detached by gentle flushing with PBS-EDTA (5 mM EDTA), recovered by centrifugation and resuspended in assay buffer (KRH: 5 mM KCl, 1.25 mM MgSO₄, 124 mM NaCl, 25 mM HEPES, 13.3 mM Glucose, 1.25 mM KH₂PO₄, 1.45 mM CaCl₂, 0.5 g/l BSA). Dose response curves were performed in parallel with the reference agonists (hADM or mADM).

Antagonist Test (96 Well):

For antagonist testing, 6 μl of the reference agonist (human (5.63 nM) or mouse (0.67 nM) adrenomedullin) was mixed with 6 μl of the test samples at different antagonist dilutions; or with 6 μl buffer. After incubation for 60 min at room temperature, 12 μl of cells (2,500 cells/well) were added. The plates were incubated for 30 min at room temperature. After addition of the lysis buffer, percentage of DeltaF will be estimated, according to the manufacturer specification, with the HTRF kit from Cis-Bio International (cat n° 62AM2 PEB) hADM 22-52 was used as reference antagonist.

Antibodies Testing cAMP-HTRF Assay:

The anti-h-ADM antibodies (NT-H, MR-H, CT-H) were tested for antagonist activity in human recombinant adrenomedullin receptor (FAST-027C) cAMP functional assay in the presence of 5.63 nM Human ADM 1-52 (SEQ ID No. 20), at the following final antibody concentrations: 100 μg/ml, 20 μg/ml, 4 μg/ml, 0.8 μg/ml, 0.16 μg/ml. The anti-m-ADM antibodies (NT-M, MR-M, CT-M) were tested for antagonist activity in human recombinant adrenomedullin receptor (FAST-027C) cAMP functional assay in the presence of 0.67 nM Mouse ADM 1-50 (SEQ ID No. 22), at the following final antibody concentrations: 100 μg/ml, 20 μg/ml, 4 μg/ml, 0.8 μg/ml, 0.16 μg/ml. Data were plotted relative inhibition vs. antagonist concentration (see FIGS. 2A to 2L). The maximal inhibition by the individual antibody is given in table 4.

TABLE 4
Maximal inhibition of ADM-antibodies

| | Maximal inhibition of ADM |
|---|---|
| Antibody | bioactivity (ADM-Bioassay) (%) |
| NT-H | 38 |
| MR-H | 73 |
| CT-H | 100 |
| NT-M FAB | 26 |
| NT-M FAB2 | 28 |
| NT-M | 45 |
| MR-M | 66 |
| CT-M | 100 |
| Non specific mouse IgG | 0 |

### Example 3—Stabilization of hADM by the Anti-ADM Antibody

The stabilizing effect of human ADM by human ADM antibodies was tested using a hADM immunoassay. The technology used was a sandwich coated tube luminescence immunoassay, based on Acridinium ester labelling.

Labelled compound (tracer): 100 μg (100 μl) CT-H (1 mg/ml in PBS, pH 7.4, AdrenoMed AG Germany) was mixed with 10 μl Acridinium NHS-ester (1 mg/ml in acetonitrile, InVent GmbH, Germany) (EP 0353971) and incubated for 20 min at room temperature. Labelled CT-H was purified by Gel-filtration HPLC on Bio-Sil® SEC 400-5 (Bio-Rad Laboratories, Inc., USA) The purified CT-H was diluted in (300 mmol/L potassium phosphate, 100 mmol/L NaCl, 10 mmol/L Na-EDTA, 5 g/L Bovine Serum Albumin, pH 7.0). The final concentration was approx. 800.000 relative light units (RLU) of labelled compound (approx. 20 ng labeled antibody) per 200 μL. Acridiniumester chemiluminescence was measured by using an AutoLumat LB 953 (Berthold Technologies GmbH & Co. KG).

Solid phase: Polystyrene tubes (Greiner Bio-One International AG, Austria) were coated (18 h at room temperature) with MR-H (AdrenoMed AG, Germany) (1.5 μg MR-H/0.3 mL 100 mmol/L NaCl, 50 mmol/L TRIS/HCl, pH 7.8). After blocking with 5% bovine serum albumin, the tubes were washed with PBS, pH 7.4 and vacuum dried.

Calibration: The assay was calibrated, using dilutions of hADM (BACHEM AG, Switzerland) in 250 mmol/L NaCl, 2 g/L Triton X-100, 50 g/L Bovine Serum Albumin, 20 tabs/L Protease Inhibitor Cocktail (Roche Diagnostics AG, Switzerland).

hADM Immunoassay: 50 μl of sample (or calibrator) was pipetted into coated tubes, after adding labeled CT-H (200 μl), the tubes were incubated for 4 h at 4° C. Unbound tracer was removed by washing 5 times (each 1 ml) with washing solution (20 mM PBS, pH 7.4, 0.1% Triton X-100). Tube-bound chemiluminescence was measured by using the LB 953 (Berthold, Germany). FIG. 3 shows a typical hADM dose/signal curve. And an hADM dose signal curve in the presence of 100 μg/mL antibody NT-H. NT-H did not affect the described hADM immunoassay.

Stability of human Adrenomedullin: Human ADM was diluted in human Citrate plasma (final concentration 10 nM) and incubated at 24° C. At selected time points, the degradation of hADM was stopped by freezing at −20° C. The incubation was performed in absence and presence of NT-H (100 μg/ml). The remaining hADM was quantified by using the hADM immunoassay described above. FIG. 4 shows the stability of hADM in human plasma (citrate) in absence and in the presence of NT-H antibody. The half-life of hADM alone was 7.8 h and in the presence of NT-H, the half-life was 18.3 h. (2.3 times higher stability).

### Example 4—Sepsis Mortality

a) Early Treatment of Sepsis

Animal model: 12-15 week-old male C57Bl/6 mice (Charles River Laboratories, Germany) were used for the study. Peritonitis had been surgically induced under light isofluran anesthesia. Incisions were made into the left upper quadrant of the peritoneal cavity (normal location of the cecum). The cecum was exposed and a tight ligature was placed around the cecum with sutures distal to the insertion of the small bowel. One puncture wound was made with a 24-gauge needle into the cecum and small amounts of cecal contents were expressed through the wound. The cecum was replaced into the peritoneal cavity and the laparotomy site was closed. Finally, animals were returned to their cages with free access to food and water. 500 μl saline were given s.c. as fluid replacement.

Application and dosage of the compound (NT-M, MR-M, CT-M): Mice were treated immediately after CLP (early treatment). CLP is the abbreviation for cecal ligation and puncture (CLP).

Study groups: Three compounds were tested versus: vehicle and versus control compound treatment. Each group contained 5 mice for blood drawing after 1 day for BUN (serum blood urea nitrogen test) determination. Ten further mice per each group were followed over a period of 4 days.

Group Treatment (10 μl/g bodyweight) dose/Follow-Up:
1 NT-M, 0.2 mg/ml survival over 4 days
2 MR-M, 0.2 mg/ml survival over 4 days
3 CT-M, 0.2 mg/ml survival over 4 days
4 non-specific mouse IgG, 0.2 mg/ml survival over 4 days
5 control—PBS 10 μl/g bodyweight survival over 4 days

Clinical chemistry: Blood urea nitrogen (BUN) concentrations for renal function were measured baseline and day 1 after CLP. Blood samples were obtained from the cavernous sinus with a capillary under light ether anaesthesia. Measurements were performed by using an AU 400 Olympus Multianalyser. The 4-day mortality and the average BUN concentrations are given in table 5.

TABLE 5
4-day mortality and BUN concentrations

| | survival | BUN pre CLP | BUN day 1 |
|---|---|---|---|
| 4-day mortality | (%) | (mM) | (mM) |
| PBS | 0 | 8.0 | 23.2 |
| non-specific mouse IgG | 0 | 7.9 | 15.5 |
| CT-M | 10 | 7.8 | 13.5 |
| MR-M | 30 | 8.1 | 24.9 |
| NT-M | 70 | 8.8 | 8.2 |

It can be seen from Table 4 that the NT-M antibody reduced mortality considerably. After 4 days 70% of the mice survived when treated with NT-M antibody. When treated with MR-M antibody 30% of the animals survived and when treated with CT-M antibody 10% of the animals survived after 4 days. In contrast thereto all mice were dead after 4 days when treated with unspecific mouse IgG. The same result was obtained in the control group where PBS (phosphate buffered saline) was administered to mice. The blood urea nitrogen or BUN test is used to evaluate kidney function, to help diagnose kidney disease, and to monitor patients with acute or chronic kidney dysfunction or failure. The results of the S-BUN Test revealed that the NT-M antibody was the most effective to protect the kidney.

b) Late Treatment of Sepsis

Animal model: 12-15 week-old male C57Bl/6 mice (Charles River Laboratories, Germany) were used for the study. Peritonitis had been surgically induced under light isofluran anesthesia. Incisions were made into the left upper quadrant of the peritoneal cavity (normal location of the cecum). The cecum was exposed and a tight ligature was placed around the cecum with sutures distal to the insertion of the small bowel. One puncture wound was made with a 24-gauge needle into the cecum and small amounts of cecal contents were expressed through the wound. The cecum was replaced into the peritoneal cavity and the laparotomy site was closed. Finally, animals were returned to their cages with free access to food and water. 500 μl saline were given s.c. as fluid replacement.

Application and dosage of the compound (NT-M FAB2): NT-M FAB2 was tested versus: vehicle and versus control compound treatment. Treatment was performed after full development of sepsis, 6 hours after CLP (late treatment). Each group contained 4 mice and were followed over a period of 4 days.

Group Treatment (10 μl/g bodyweight) dose/Follow-Up:
1 NT-M, FAB2 0.2 mg/ml survival over 4 days
2 control non-specific mouse IgG, 0.2 mg/ml survival over 4 days
3 vehicle:—PBS 10 μl/g bodyweight survival over 4 days

TABLE 6
4-day mortality

| 4 day mortality | survival (%) |
|---|---|
| PBS | 0 |
| Non-specific mouse IgG | 0 |
| NT-M FAB2 | 75 |

It can be seen from Table 6 that the NT-M FAB 2 antibody reduced mortality considerably. After 4 days 75% of the mice survived when treated with NT-M FAB 2 antibody. In contrast thereto all mice were dead after 4 days when treated with non-specific mouse IgG. The same result was obtained in the control group where PBS (phosphate buffered saline) was administered to mice.

### Example 5—Administration of NT-H in Healthy Humans

The study was conducted in healthy male subjects as a randomized, double-blind, placebo-controlled, study with single escalating doses of NT-H antibody administered as intravenous (i.v.) infusion in 3 sequential groups of 8 healthy male subjects each (1st group 0.5 mg/kg, 2nd group 2 mg/kg, 3rd group 8 mg/kg) of healthy male subjects (n=6 active, n=2 placebo for each group). The main inclusion criteria were written informed consent, age 18-35 years, agreement to use a reliable way of contraception and a BMI between 18 and 30 kg/m². Subjects received a single i.v. dose of NT-H antibody (0.5 mg/kg; 2 mg/kg; 8 mg/kg) or placebo by slow infusion over a 1-hour period in a research unit. The baseline ADM-values in the 4 groups did not differ. Median ADM values were 7.1 pg/mL in the placebo group, 6.8 pg/mL in the first treatment group (0.5 mg/kg), 5.5 pg/mL in second treatment group (2 mg/kg) and 7.1 pg/mL in the third treatment group (8 mg/mL). The results show that ADM-values rapidly increased within the first 1.5 hours after administration of NT-H antibody in healthy human individuals, then reached a plateau and slowly declined (FIG. 6).

### Example 6—Bio-ADM in Patients Infected with Corona Virus (SARS-CoV-2)

Plasma samples from 12 patients that were diagnosed of being infected with Corona virus (SARS-CoV-2) were screened for bio-ADM. Bio-ADM levels were measured using an immunoassay as described in Weber et al. 2017 (Weber et al. 2017. JALM 2(2): 222-233). In addition, DPP3-levels were measured using an immunoassay (LIA) as described recently (Rehfeld et al. 2019. JALM 3(6): 943-953). The respective bio-ADM and DPP3 concentrations in individual samples are summarized in table 7.

TABLE 7
bio-ADM and DPP3 levels in samples from patients
infected with Corona virus (SARS-CoV-2)

| Patient No. | DPP3 (ng/ml) | bio-ADM (pg/ml) |
|---|---|---|
| 1 | 56 | 133 |
| 2 | 30 | 45 |
| 3 | 70 | 214 |
| 4 | 150 | 85 |
| 5 | 290 | 437 |
| 6 | 87 | 66 |
| 7 | 975 | 79 |
| 8 | 333 | 174 |
| 9 | 216 | 35 |
| 10 | 539 | 199 |
| 11 | 27 | 53 |
| 12 | 162 | 401 |
| Median | 156.0 | 109.0 |
| mean | 244.6 | 160.1 |

Bio-ADM concentrations in samples from patients infected with Corona virus (SARS-CoV-2) ranged between 35 and 437 pg/ml with a median (IQR) of 109 (56-210) pg/ml. Median plasma bio-ADM (mature ADM-NH₂) in samples from (healthy) subjects was 24.7 pg/ml, the lowest value 11 pg/ml and the 99^{th} percentile 43 pg/ml (Marino et al. 2014. Critical Care 18:R34). Bio-ADM in patients infected with Corona virus (SARS-CoV-2) were significantly elevated compared to healthy controls.

DPP3 concentrations ranged between 27 and 975 ng/ml with a median (IQR) of 156.0 (59.5-322.3) ng/ml. DPP3 concentrations are significantly elevated compared to healthy subjects. Samples from 5,400 normal (healthy) subjects (swedish single-center prospective population-based Study (MPP-RES)) have been measured: median (interquartile range) plasma DPP3 was 14.5 ng/ml (11.3 ng/ml-19 ng/ml).

### Example 7—Change of Lung Function Under NT-ADM Antibody Treatment in Patients with Compromised Lung Function (AdrenOSS-2)

AdrenOSS-2 is a double-blind, placebo-controlled, randomized, multicenter, proof of concept and dose-finding phase II clinical trial to investigate the safety, tolerability and efficacy of the N-terminal ADM antibody named Adrecizumab in patients with septic shock and elevated adrenomedullin (Geven et al. BMJ Open 2019; 9:e024475). In total, 301 patients with septic shock and bio-ADM concentration >70 pg/mL were randomized (2:1:1) to treatment with a single intravenous infusion over approximately 1 hour with either placebo (n=152), adrecizumab 2 ng/kg (n=72) or Adrecizumab 4 ng/kg (n=77). All-cause mortality within 28 (90) days after inclusion was 25.8% (34.8%). Mean age was 68.4 years and 61% were male. For the per protocol analysis, n=294 patients remained eligible, and 14-day all-cause mortality rate was 18.5%.

In patients treated with Adrecizumab (both doses combined, per protocol population), a trend to lower short-term mortality (14 days post admission) was observed compared to placebo (Hazard ratio (HR) 0.701 [0.408-1.21], p=0.200).

Furthermore, different subpopulations of the cohort were analyzed. Main outcomes were 28-day mortality, change in Horovitz Index (PaO₂/FiO₂) (at 24 h/48 h/72 h), change in SOFA score (at 24 h/48 h/72 h) or change in respiratory SOFA score component (also based on PaO₂/FiO₂) (at 24 h/48 h/72 h). All p-values are 2-sided and a p-value of 0.20 should be considered significant.

A subpopulation of shock patients who met the following criteria: Horovitz-Index of <170 and mechanical ventilation at baseline (n=48) was analyzed. This group mimics critically-ill Covid-19 patients on the ICU and in need for mechanical ventilation. 28-day mortality trended to be lower in patients treated with Adrecizumab compared to placebo (p=0.37) (FIG. 7). The change in Horovitz-Index was significantly higher after 48 (p=0.09) and 72 hours (p=0.11) (FIGS. 8 B and C), with a mean increase of 64.2 and 66.4, respectively, and trended to be higher after 24 hours (0.48) (FIG. 8 A) in patients treated with Adrecizumab. The change in SOFA score was significantly lower after 24 hours (p=0.032), 48 (p=0.012) and 72 hours (p=0.028) (FIGS. 9 A, B and C), respectively, in patients treated with Adrecizumab when compared to the placebo group.

A subpopulation of shock patients with ALI/ARDS which was defined via respiratory physical examination on admission (n=80) was further analyzed. The change in SOFA score was significantly lower after 24 hours (p=0.005) and 48 (p=0.025) (FIG. 10 A, B), respectively, and trended to be lower after 72 hours (p=0.38) (FIG. 10 C) in patients treated with Adrecizumab when compared to the placebo group. Moreover, the change in respiratory SOFA score in patients with ALI/ARDS was significantly lower after 48 hours (p=0.09) and trended to be lower after 24 hours (p=0.26) (FIG. 11 A, B), respectively, in patients treated with Adrecizumab when compared to the placebo group.

Another subpopulation of shock patients was selected meeting the criterion of mechanical ventilation at baseline (n=161). 28-day mortality was significantly lower in patients treated with Adrecizumab compared to placebo (p=0.157) (FIG. 12). The change in Horovitz-Index was significantly higher after 24 hours (p=0.155), 48 hours (p=0.007) and 72 hours (p=0.087) (FIGS. 13 A, B and C), respectively, with a mean increase of 56.2 at 24 hours, in patients treated with Adrecizumab. Moreover, the change in SOFA score in patients with mechanical ventilation at baseline was significantly lower after 24 hours (p=0.002) and 48 hours (p=0.109) and trended to be lower after 72 hours (p=0.31) (FIGS. 14 A, B and C), respectively, in patients treated with Adrecizumab when compared to the placebo group. Particularly, the change in respiratory SOFA score in patients with mechanical ventilation at baseline was significantly lower after 24 hours (p=0.021), 48 hours (p=0.011) and 72 hours (p=0.066) (FIGS. 15 A, B and C), respectively, in patients treated with Adrecizumab when compared to the placebo group. These data strongly support that NT-ADM antibodies are capable to improve endothelial function and vascular integrity in critically ill patients with compromised lung function and suggest its applicability to COVID-19 patients.

### Example 8—Prognostic Value of Bio-ADM in Critically Ill Patients with COVID-19

The aim of this study was to determine if bioactive adrenomedullin (bio-ADM) can assist in the risk stratification and clinical management of critically ill COVID-19 patients.

8.1. Study Population and Data Collection

After ethical approval (Ethical Committee of RWTH University, EK 100/20), this prospective observational study was performed between Mar. 13 and Apr. 16, 2020 at the University Hospital RWTH Aachen, Germany. All patients or their legal representatives provided written informed consent. All patients with positive SARS-CoV-2 PCR results and ICU admission were included in this study. The exclusion criteria were age <18 years old, pregnancy, and palliative care. The analysis was carried out using real time reverse transcription PCR (RT-PCR). Treatment of patients followed the standards of care in our ICU, including mechanical ventilation, veno-venous ECMO, and RRT and norepinephrine if needed. Decision on the use of veno-venous ECMO therapy was based on the recently published Extracorporeal Life Support Organization (ELSO) consensus guideline (Bartlett et al. 2020. ASAIO Journal 66: 472-474). All parameters including demographics, vital signs, laboratory values, blood gas analyses and organ support have been extracted from the patient data management system (Intellispace Critical Care and Anesthesia (ICCA) system, Philips, Netherlands).

8.2. Bio-ADM Measurement

Blood was sampled on the day of admission and on a daily basis until day 7 for analysis of bio-ADM and standard laboratory parameters. Bio-ADM was measured in EDTA plasma with a one-step luminescence sandwich immunoassay (Weber et al. 2017. JALM 2(2): 222-233). In brief, 100 sample were incubated under agitation for one hour at room temperature with 150 μL detection antibody directed against the C-terminus of bio-ADM in a microtiter plate coated with monoclonal antibody directed against mid-regional bio-ADM. Synthetic human bio-ADM was used as calibrator. After washing, the chemiluminescence signal was measured in a microtiter plate luminescence reader (Centro LB960, Berthold Technologies, Bad Wildbad, Germany). The assay had a lower detection limit of 3 pg/mL. In a reference population of 200 healthy individuals, median (99th percentile) bio-ADM levels were 20.7 pg/mL (43 pg/mL) (Marino et al. 2014. Critical Care 18: R34).

8.3. Statistics

Values are expressed as medians and interquartile ranges (IQR), or counts and percentages, as appropriate. Group comparisons of continuous variables were performed using Kruskal-Wallis test. Categorical data were compared using Pearson's Chi-squared Test for Count Data. Biomarker data were log-transformed. Boxplots were used to illustrate differences of bio-ADM in categorical variables. Cox proportional-hazards regression was used to analyze the effect of (log-transformed) bio-ADM on survival in univariable analyses. The assumptions of proportional hazard were tested. The predictive value of a model was assessed by the model likelihood ratio Chi-square statistic. The concordance index (C index) is given as an effect measure. It is equivalent to the concept of AUC adopted for binary outcome. Survival curves plotted by the Kaplan-Meier method were used for illustrative purposes. All statistical tests were 2-tailed and a two-sided p-value of 0.05 was considered for significance. The statistical analyses were performed using R version 3.4.3 (http://www.r-project.org, library rms, Hmisc, ROCR) and Statistical Package for the Social Sciences (SPSS) version 22.0 (SPSS Inc., Chicago, Ill., USA).

8.4. Results

In this cohort study, 53 patients with COVID-19 were consecutively included after confirmed SARS-CoV-2 infection and the need of ICU admission (n=40 male [76%], median [IQR] age 62 [57-70] years) (Table 7). Median ICU length of stay was 16 (7.5-20) days. 32 patients (60%) were discharged from ICU to normal ward prior to day 28, while 8 patients (15%) remained in the ICU and 13 patients (25%) died. Markers of systemic inflammation are shown in Table 7.

TABLE 7
Baseline characteristics in 53 COVID-19 critically ill patients

| Variable | all | | none (n = 3) | | mild (n = 12) | |
|---|---|---|---|---|---|---|
| Age (years) | 62 | [57-70] | 53 | [49-65] | 61 | [59-64] |
| Gender male, n (%) | 40 | (75.5) | 3 | (100) | 10 | (83.3) |
| Body mass index (kg/m²) | 29.3 | [24.9-32.6] | 24.9 | [24.7-28.2] | 29.2 | [26.3-34.9] |
| Temperature, max (° C.) | 38.1 | [37.4-38.5] | 38.1 | [37.8-38.8] | 38.1 | [37.8-38.6] |
| Heart rate (bpm) | 106 | [89-114] | 93 | [86-107] | 105 | [93-109] |
| Respiratory rate (bpm) | 25 | [23-28] | 24 | [22-25] | 24 | [23-26] |
| SOFA score at day of enrollment (points) | 9 | [7-11] | 8.5 | [7.75-9.25] | 7 | [6-9.5] |

Blood gas analysis (at day of enrollment)

| | | | | | | |
|---|---|---|---|---|---|---|
| Arterial pH | 7.36 | [7.3-7.42] | 7.47 | [7.38-7.49] | 7.4 | [7.37-7.44] |
| pCO₂ (mmHg) | 45.05 | [39.25-52.02] | 48 | [42.05-71.3] | 36.7 | [33.75-41.2] |
| pO₂ (mmHg) | 79 | [70-91] | 71 | [63.5-79.5] | 92 | [75-104.5] |
| SpO₂ (%) | 95 | [94-98.25] | 94 | [93.5-94.5] | 98 | [96-99] |
| Horowitz index (mmHg/%) | 113.5 | [87.5-151.25] | 133 | [88.5-276] | 224 | [167.5-275.5] |

Biomarker (at day of enrollment, unless stated differently)

| | | | | | | |
|---|---|---|---|---|---|---|
| bio-ADM (pg/mL) | 59.9 | [37.9-101.9] | 28.3 | [19.9-28.4] | 39.0 | [29.2-54.5] |
| bio-ADM > 70 pg/mL, n (%) | 22 | (41.5) | 0 | (0) | 1 | (8.3) |
| lactate (mmol/L) | 1 | [0.8-1.42] | 0.7 | [0.5-0.95] | 0.8 | [0.7-0.9] |
| IL-6 (pg/mL) | 158.4 | [97.42-337.4] | 51.95 | [34.52-69.39] | 65.73 | [46.88-93.52] |
| PCT (ng/mL) | 0.53 | [0.13-1.89] | 0.07 | [0.06-0.08] | 0.14 | [0.11-0.25] |
| CRP (nmol/L) | 174.6 | [117.4-325.62] | 182.1 | [182.1-182.1] | 79.5 | [34.25-142.43] |
| WBC (10³/mm³) | 9.3 | [6.6-13] | 10.4 | [9.3-11.85] | 6.15 | [5.07-10.82] |
| Platelets (10³/μL) | 228 | [198-329] | 202 | [200-292] | 197 | [139.75-235.75] |
| Creatinine (mg/dL) | 1.06 | [0.76-2.18] | 0.67 | [0.6-0.72] | 0.96 | [0.76-1.21] |

Comorbidities

| | | | | | | |
|---|---|---|---|---|---|---|
| Arterial hypertension, n (%) | 27 | (50.9) | 1 | (33.3) | 5 | (41.7) |
| Diabetes mellitus, n (%) | 13 | (24.5) | 0 | (0) | 1 | (8.3) |
| Ischemic heart disease, n (%) | 10 | (18.9) | 0 | (0) | 2 | (16.7) |
| Embolism/Thrombosis, n (%) | 6 | (11.3) | 1 | (33.3) | 1 | (8.3) |
| Cardiac arrhythmia, n (%) | 6 | (11.3) | 0 | (0) | 1 | (8.3) |
| Cerebral vascular disease, n (%) | 5 | (9.4) | 0 | (0) | 2 | (16.7) |
| COPD, n (%) | 6 | (11.3) | 1 | (33.3) | 2 | (16.7) |
| Other lung diseases, n (%) | 2 | (3.8) | 1 | (33.3) | 1 | (8.3) |
| Chronic kidney disease, n (%) | 8 | (15.1) | 0 | (0) | 2 | (16.7) |
| Tumor disease, n (%) | 4 | (7.5) | 0 | (0) | 3 | (25) |
| Smoker, n (%) | 3 | (5.7) | 0 | (0) | 1 | (8.3) |

Treatment on ICU (first 14 days, unless stated differently)

| | | | | | | |
|---|---|---|---|---|---|---|
| ICU length of stay (days) | 16 | [7.5-20] | 6 | [4-9.5] | 7.5 | [3-10.5] |
| Highest dose of Norepinephrine during | 0.15 | [0.06-0.29] | 0.07 | [0.03-0.11] | 0 | [0-0.09] |
| the first 7 days (μg/kg/min) | | | | | | |
| Anticoagulation, n (%) | 15 | (28.3) | 1 | (33.3) | 2 | (16.7) |
| Antiplatelet, n (%) | 15 | (28.3) | 0 | (0) | 4 | (33.3) |
| Antihypertensiva, n (%) | 32 | (60.4) | 1 | (33.3) | 8 | (66.7) |
| Immunsupressant, n (%) | 9 | (17) | 1 | (33.3) | 2 | (16.7) |
| Analgesics, n (%) | 8 | (15.1) | 1 | (33.3) | 4 | (33.3) |

Outcome

| | | | | | | |
|---|---|---|---|---|---|---|
| Death 28 days, n (%) | 13 | (24.5) | 1 | (33.3) | 0 | (0) |
| Disposition on day 28 | | | | | | |
| discharged, n (%) | 32 | (60.4) | 2 | (66.7) | 12 | (100) |
| on ICU post day 28, n (%) | 8 | (15.1) | 0 | (0) | 0 | (0) |
| Death 28 days, n (%) | 13 | (24.5) | 1 | (33.3) | 0 | (0) |

| Variable | moderate (n = 13) | | severe (n = 25) | | p-value |
|---|---|---|---|---|---|
| Age (years) | 62 | [54-67] | 66 | [58-72] | 0.767 |
| Gender male, n (%) | 6 | (46.2) | 21 | (84) | 0.0385 |
| Body mass index (kg/m²) | 30.5 | [26.7-35.2] | 29.3 | [24.7-31.3] | 0.758 |
| Temperature, max (° C.) | 38.2 | [37.0-38.5] | 38.0 | [37.3-38.5] | 0.934 |
| Heart rate (bpm) | 91 | [72-103] | 112 | [104-121] | 0.014 |
| Respiratory rate (bpm) | 25 | [22-28] | 25 | [23-29] | 0.678 |
| SOFA score at day of enrollment (points) | 8.5 | [7.75-10] | 11 | [9-11] | 0.037 |
| Blood gas analysis (at day of enrollment) | | | | | |
| Arterial pH | 7.38 | [7.33-7.43] | 7.32 | [7.28-7.36] | 0.011 |
| pCO₂ (mmHg) | 45.5 | [43.2-52] | 48.2 | [42.1-55.4] | 0.001 |
| pO₂ (mmHg) | 79 | [70-92] | 79 | [70-84] | 0.345 |
| SpO₂ (%) | 98 | [95-100] | 94 | [93-97] | 0.031 |
| Horowitz index (mmHg/%) | 115 | [100-150] | 94 | [71-115] | 0.002 |
| Biomarker (at day of enrollment, unless stated differently) | | | | | |
| bio-ADM (pg/mL) | 48.1 | [26.9-79.8] | 101.9 | [67.0-201.1] | <0.001 |
| bio-ADM > 70 pg/mL, n (%) | 4 | (30.8) | 17 | (68.0) | 0.002 |
| lactate (mmol/L) | 0.9 | [0.7-1.5] | 1.3 | [1-1.7] | 0.003 |
| IL-6 (pg/mL) | 211.25 | [141.27-519.95] | 251.5 | [151.2-475.25] | 0.001 |
| PCT (ng/mL) | 0.22 | [0.11-0.69] | 1.46 | [0.66-5.06] | <0.001 |
| CRP (nmol/L) | 256.3 | [124.1-298.4] | 251.15 | [158.4-350] | 0.002 |
| WBC (10³/mm³) | 8 | [7.4-9.4] | 10.1 | [8-13.9] | 0.120 |
| Platelets (10³/μL) | 237 | [204-328] | 263 | [204-338] | 0.242 |
| Creatinine (mg/dL) | 0.89 | [0.6-1.08] | 1.79 | [1.18-3.02] | 0.004 |
| Comorbidities | | | | | |
| Arterial hypertension, n (%) | 9 | (69.2) | 12 | (48) | 0.455 |
| Diabetes mellitus, n (%) | 3 | (23.1) | 9 | (36) | 0.215 |
| Ischemic heart disease, n (%) | 4 | (30.8) | 4 | (16) | 0.557 |
| Embolism/Thrombosis, n (%) | 3 | (23.1) | 1 | (4) | 0.197 |
| Cardiac arrhythmia, n (%) | 0 | (0) | 5 | (20) | 0.259 |
| Cerebral vascular disease, n (%) | 0 | (0) | 3 | (12) | 0.459 |
| COPD, n (%) | 1 | (7.7) | 2 | (8) | 0.525 |
| Other lung diseases, n (%) | 0 | (0) | 0 | (0) | 0.025 |
| Chronic kidney disease, n (%) | 3 | (23.1) | 3 | (12) | 0.708 |
| Tumor disease, n (%) | 1 | (7.7) | 0 | (0) | 0.057 |
| Smoker, n (%) | 2 | (15.4) | 0 | (0) | 0.247 |
| Treatment on ICU (first 14 days, unless stated differently) | | | | | |
| ICU length of stay (days) | 19.5 | [16.5-22.75] | 17.5 | [15-20.75] | 0.004 |
| Highest dose of Norepinephrine during | 0.15 | [0.06-0.18] | 0.29 | [0.13-0.35] | <0.001 |
| the first 7 days (μg/kg/min) | | | | | |
| Anticoagulation, n (%) | 3 | (23.1) | 9 | (36) | 0.627 |
| Antiplatelet, n (%) | 6 | (46.2) | 5 | (20) | 0.238 |
| Antihypertensiva, n (%) | 10 | (76.9) | 13 | (52) | 0.343 |
| Immunsupressant, n (%) | 4 | (30.8) | 2 | (8) | 0.289 |
| Analgesics, n (%) | 1 | (7.7) | 2 | (8) | 0.143 |
| Outcome | | | | | |
| Death 28 days, n (%) | 1 | (7.7) | 11 | (44) | 0.011 |
| Disposition on day 28 | | | | | 0.001 |
| discharged, n (%) | 11 | (84.6) | 7 | (28) | |
| on ICU post day 28, n (%) | 1 | (7.7) | 7 | (28) | |
| Death 28 days, n (%) | 1 | (7.7) | 11 | (44) | |

Variables are given as median [interquartile range] or number (%). ARDS, acute respiratory distress syndrome; bio-ADM, bioactive adrenomedullin; COPD, chronic obstructive pulmonary disease; CRP, C-reactive protein; ECMO, extracorporeal membrane oxygenation; FiO2, fraction of inspired oxygen; ICU, intensive care unit; IL-6, interleukin-6; pCO2, partial pressure of carbon dioxide; PCT, procalcitonin; PEEP, positive end-expiratory pressure; pO2, partial pressure of oxygen; RRT, renal replacement therapy; spO2, peripheral capillary oxygen saturation; SOFA, sequential organ failure assessment; WBC, white blood cell counts A high proportion of 38 patients (72%) presented with moderate or severe ARDS (25% moderate, 47% severe). Bio-ADM levels increased with severity of ARDS (p<0.001, bio-ADM 28.3 [19.9-28.4], 39.0 [29.2-54.5], 48.1 [26.9-79.8] and 101.9 [67.0-201.1] pg/mL compared to patients without ARDS, mild ARDS, moderate ARDS or severe ARDS, respectively) (FIG. 16).

The majority of patients (n=44) received invasive ventilation during ICU stay (Table 7). Bio-ADM levels were significantly increased in invasively ventilated patients compared to spontaneously breathing patients (68.2 [45.5-106.6] pg/mL vs. 31.8 [18.6-48.4] pg/mL, p=0.006) (FIG. 17A). Of note, bio-ADM levels were similarly elevated in patients who received invasive ventilation upon enrollment (n=38) compared to those patients who required mechanical ventilation in due course during the study period (n=6) (69.8 [44.1-107.3] pg/mL vs. 63.2 [51.0-88.7] pg/mL).

Increased bio-ADM levels were observed in patients treated with veno-venous ECMO (n=9), compared to patients without ECMO therapy (101.9 [65.0-144.1] pg/mL vs. 53.3 [29.2-91.0] pg/mL, p=0.040) (FIG. 17B). Notably, highest bio-ADM levels were observed in patients who were eligible for ECMO therapy due to severity of respiratory failure according to the ELSO consensus guideline (n=7) (Bartlett et al. 2020. ASAIO Journal 66: 472-474), but were not treated with ECMO due to individual patient decree (262.1 [136.1-274.6] pg/mL, p<0.001) (FIG. 17B). Moreover, bio-ADM levels significantly correlated with the dose of norepinephrine (r=0.47, p<0.001). Patients without norepinephrine had the lowest bio-ADM levels (n=15, median 37.9 pg/mL), patients with a low norepinephrine dose (up to 0.1 μg/kg/min, n=15) had slightly elevated bio-ADM levels (median 53.8 pg/mL), and patients with a high norepinephrine dose (>0.1 μg/kg/min, n=23) had the highest bio-ADM levels (median 105.9 pg/mL, p=0.002).

With respect to kidney function, there was a notable correlation between bio-ADM and serum creatinine (r=0.62, p<0.001). In line, significantly higher bio-ADM levels were found in patients receiving RRT (n=27) compared to patients without RRT (n=26) (101.9 [67.7-182.9] pg/mL vs. 40.2 [27.2-53.5] pg/mL, p<0.001) (FIG. 17C).

Bio-ADM levels were higher in non-survivors (n=13) than survivors (n=40) (107.6 [51.0-262.1] pg/mL vs. 53.3 [29.2-91.0] pg/mL, p=0.010). Notably, bio-ADM predicted 28-day mortality (C-index 0.72, 95% confidence interval [CI] 0.56-0.87, p<0.001) (FIG. 18A). We next elucidated the additional value of serial measurement of bio-ADM for the prediction of 28-day mortality. Based on previous studies (Mebazaa et al. 2018. Critical Care 22: 354), we applied a cut-off value for bio-ADM of 70 pg/mL and grouped the patients accordingly. Patients presenting bio-ADM levels of above 70 pg/mL on enrollment (high) and remaining above that value (high-high) showed the worst outcome, while patients that improve within 48 h (high-low) showed a favorable outcome. Likewise, patients displaying an increase in bio-ADM (low-high) at 48 h showed an unfavorable outcome (FIG. 18B).

In conclusion, bio-ADM plasma levels correlate with the disease severity, need for extracorporeal organ assist, and outcome highlighting the promising value of bio-ADM in the early risk stratification and management of patients with COVID-19. Moreover, the data clearly highlight the role of endothelial dysfunction in the pathophysiology of COVID-19 and open up for future randomized trials that prospectively evaluate bio-ADM as a new objective tool for risk stratification and monitoring of patients suffering from COVID-19.

### FIGURE DESCRIPTION

FIG. 1A:

Illustration of antibody formats—Fv and scFv-Variants.

FIG. 1B:

Illustration of antibody formats—heterologous fusions and bifunctional antibodies.

FIG. 1C:

Illustration of antibody formats—bivalental antibodies and bispecific antibodies.

FIG. 2A: Dose response curve of human ADM. Maximal cAMP stimulation was adjusted to 100% activation.

FIG. 2B: Dose/inhibition curve of human ADM 22-52 (ADM-receptor antagonist) in the presence of 5.63 nM hADM.

FIG. 2C: Dose/inhibition curve of CT-H in the presence of 5.63 nM hADM.

FIG. 2D: Dose/inhibition curve of MR-H in the presence of 5.63 nM hADM.

FIG. 2E: Dose/inhibition curve of NT-H in the presence of 5.63 nM hADM.

FIG. 2F: Dose response curve of mouse ADM. Maximal cAMP stimulation was adjusted to 100% activation.

FIG. 2G: Dose/inhibition curve of human ADM 22-52 (ADM-receptor antagonist) in the presence of 0.67 nM mADM.

FIG. 2H: Dose/inhibition curve of CT-M in the presence of 0.67 nM mADM.

FIG. 2I—Dose/inhibition curve of MR-M in the presence of 0.67 nM mADM.

FIG. 2J: Dose/inhibition curve of NT-M in the presence of 0.67 nM mADM.

FIG. 2K: Shows the inhibition of ADM by F(ab)2 NT-M and by Fab NT-M.

FIG. 2L: shows the inhibition of ADM by F(ab)2 NT-M and by Fab NT-M.

FIG. 3:

This figure shows a typical hADM dose/signal curve. And an hADM dose signal curve in the presence of 100 μg/mL antibody NT-H.

FIG. 4:

This figure shows the stability of hADM in human plasma (citrate) in absence and in the presence of NT-H antibody.

FIG. 5:

Alignment of the Fab with homologous human framework sequences.

FIG. 6: ADM-concentration in healthy human subjects after NT-H application at different doses up to 60 days.

FIG. 7: 28-day mortality in patients with Horovitz-Index of <170 and mechanical ventilation at baseline (n=48)

FIG. 8: Change in Horovitz index in patients with Horovitz-Index of <170 and mechanical ventilation at baseline (n=48) after 24 hours (A), 48 hours (B) and 72 hours (C), respectively.

FIG. 9: Change in SOFA score in patients with Horovitz-Index of <170 and mechanical ventilation at baseline (n=48) after 24 hours (A), 48 hours (B) and 72 hours (C), respectively.

FIG. 10: Change in SOFA score in patients with ALI/ARDS (n=80) after 24 hours (A), 48 hours (B) and 72 hours (C), respectively.

FIG. 11: Change in respiratory SOFA score in patients with ALI/ARDS (n=80) after 24 hours (A) and 48 hours (B), respectively.

FIG. 12: 28-day mortality in patients with mechanical ventilation at baseline (n=161) after 24 hours (A), 48 hours (B) and 72 hours (C), respectively.

FIG. 13: Change in Horovitz index in patients with mechanical ventilation at baseline (n=161) after 24 hours (A), 48 hours (B) and 72 hours (C), respectively.

FIG. 14: Change in SOFA score in patients with mechanical ventilation at baseline (n=161) after 24 hours (A), 48 hours (B) and 72 hours (C), respectively.

FIG. 15: Change in respiratory SOFA score in patients with mechanical ventilation at baseline (n=161) after 24 hours (A), 48 hours (B) and 72 hours (C), respectively.

FIG. 16: Boxplot of bio-ADM by ARDS in 53 COVID-19 patients (p<0.001). Horizontal line at 70 pg/ml.

FIG. 17: Boxplot of bio-ADM levels by invasive ventilation (A, p=0.006), ECMO (B, p<0.001) and RRT (C, p<0.001) in 53 COVID-19 patients. Horizontal line at 70 pg/mL. Patients that fulfilled the criteria for ECMO therapy, but did not receive ECMO treatment are termed “indicated”.

FIG. 18: Kaplan-Meier plot for 28-day mortality for bio-ADM. (A) Curves are plotted by bio-ADM quartiles (for continuous bio-ADM: standardized HR 3.5 (95% CI 1.6-7.5), c index 0.72 (95% CI 0.56-0.87), p<0.001). (B) Curves are plotted to illustrate the potential value of serial measurements of bio-ADM, by > or <70 pg/mL at enrollment and 48 h (right, p=n.s.). Patients with bio-ADM above 70 pg/mL upon enrollment that remain above that value (high-high) have the worst outcome, while patients that improve within 48 h (high-low) have a favorable outcome. Likewise, patients displaying an increase in bio-ADM (low-high) have an unfavorable outcome. Patients with missing bio-ADM data at 48 h remain in their initial category.

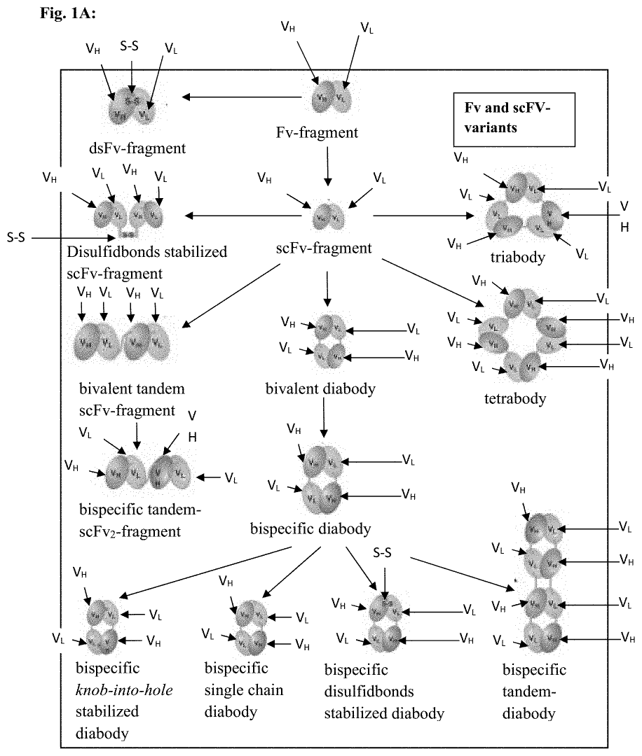

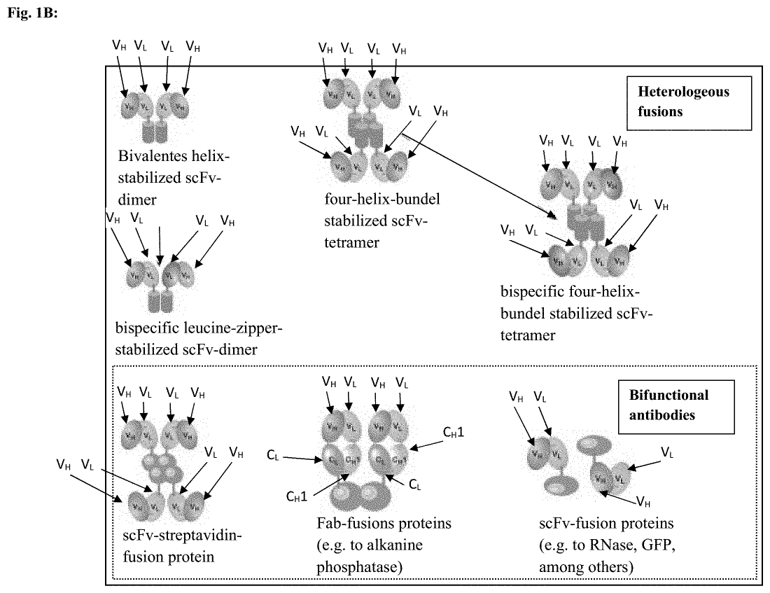

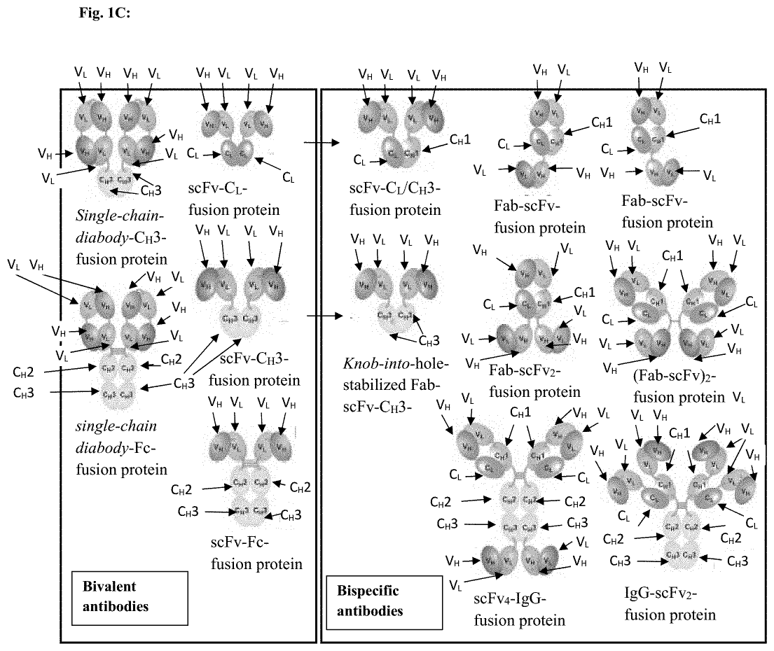

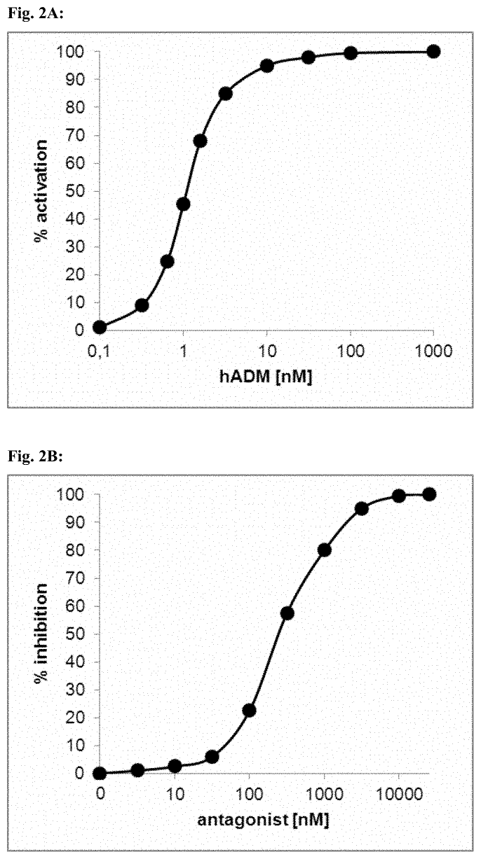

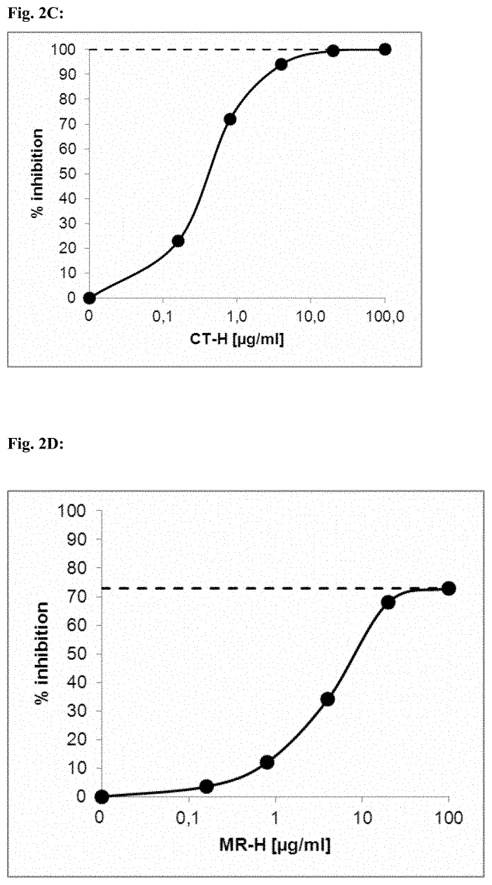

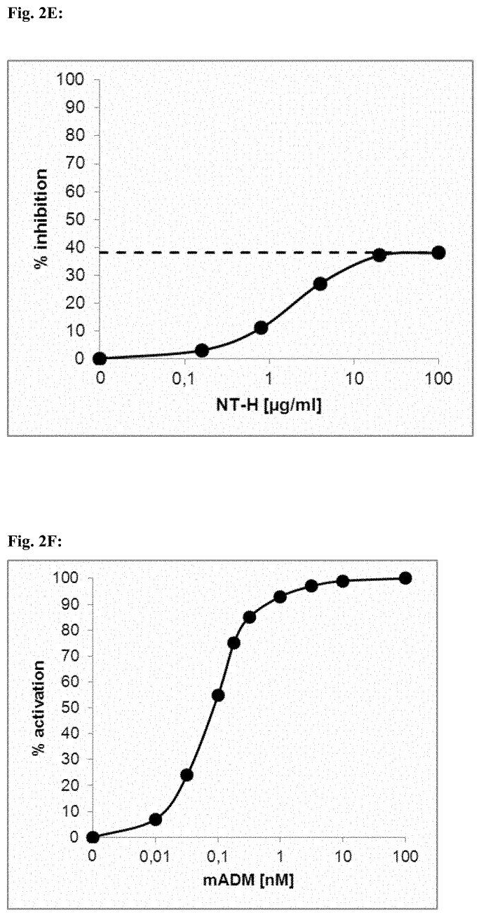

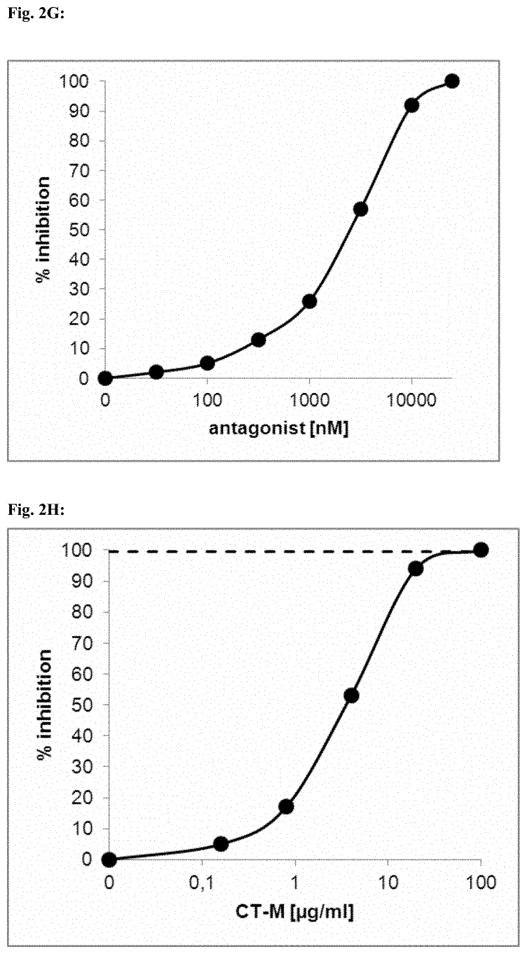

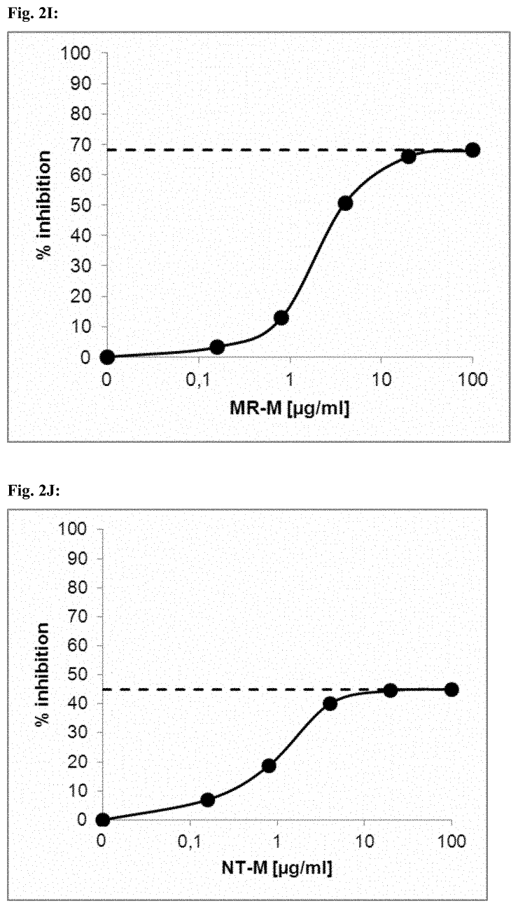

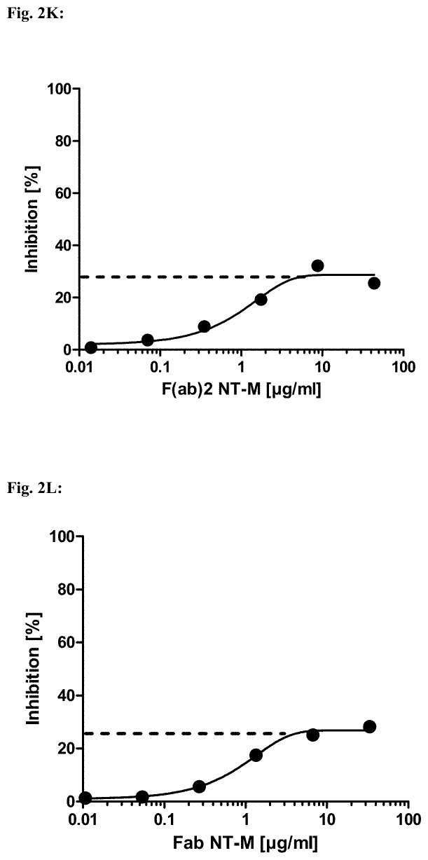

SEQUENCES
SEQ ID No.: 1
GYTFSRYW
SEQ ID No.: 2
ILPGSGST
SEQ ID No.: 3
TEGYEYDGFDY
SEQ ID No.: 4
QSIVYSNGNTY
SEQUENCE “RVS” (not part of the Sequencing
Listing):
RVS
SEQ ID No.: 5
FQGSHIPYT
SEQ ID No.: 6 (AM-VH-C)
QVQLQQSGAELMKPGASVKISCKATGYTFSRYWIEWVKQRPGHGLEWIGE
ILPGSGSTNYNEKFKGKATITADTSSNTAYMQLSSLTSEDSAVYYCTEGY
EYDGFDYWGQGTTLTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPK
SEQ ID No.: 7 (AM-VH1)
QVQLVQSGAEVKKPGSSVKVSCKASGYTFSRYWISWVRQAPGQGLEWMGR
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPK
SEQ ID No.: 8 (AM-VH2-E40)
QVQLVQSGAEVKKPGSSVKVSCKASGYTFSRYWIEWVRQAPGQGLEWMGR
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPK
SEQ ID No.: 9 (AM-VH3-T26-E55)
QVQLVQSGAEVKKPGSSVKVSCKATGYTFSRYVVISWVRQAPGQGLEWMG
EILPGSGSTNYAQKFQGRVTITADESTSTAYMELSSLRSEDTAVYYCTEG
YEYDGFDYWGQGTTVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPK
SEQ ID No.: 10 (AM-VH4-T26-E40-E55)
QVQLVQSGAEVKKPGSSVKVSCKATGYTFSRYVVIEWVRQAPGQGLEWMG
EILPGSGSTNYAQKFQGRVTITADESTSTAYMELSSLRSEDTAVYYCTEG
YEYDGFDYWGQGTTVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPK
SEQ ID No.: 11 (AM-VL-C)
DVLLSQTPLSLPVSLGDQATISCRSSQSIVYSNGNTYLEWYLQKPGQSPK
LLIYRVSNRFSGVPDRFSGSGSGTDFTLKISRVEAEDLGVYYCFQGSHIP
YTFGGGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
SEQ ID No.: 12 (AM-VL1)
DVVMTQSPLSLPVTLGQPASISCRSSQSIVYSNGNTYLNWFQQRPGQSPR
RLIYRVSNRDSGVPDRFSGSGSGTDFTLKISRVEAEDVGVYYCFQGSHIP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
SEQ ID No.: 13 (AM-VL2-E40)
DVVMTQSPLSLPVTLGQPASISCRSSQSIVYSNGNTYLEWFQQRPGQSPR
RLIYRVSNRDSGVPDRFSGSGSGTDFTLKISRVEAEDVGVYYCFQGSHIP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
SEQ ID No.: 14 (human ADM 1-21)
YRQSMNNFQGLRSFGCRFGTC
SEQ ID No.: 15 (human ADM 21-32)
CTVQKLAHQIYQ
SEQ ID No.: 16 (human ADM C-42-52)
CAPRSKISPQGY-CONH₂
SEQ ID No.: 17 (murine ADM 1-19)
YRQSMNQGSRSNGCRFGTC
SEQ ID No.: 18 (murine ADM 19-31)
CTFQKLAHQIYQ
SEQ ID No.: 19 (murine ADM C-40-50)
CAPRNKISPQGY-CONH₂
SEQ ID No.: 20 (mature human Adrenomedullin
(mature ADM); amidated ADM; bio-ADM): amino
acids 1-52 or amino acids 95-146 of pro-ADM
YRQSMNNFQGLRSFGCRFGTCTVQKLAHQIYQFTDKDKDNVAPRSKISPQ
GY-CONH₂
SEQ ID No.: 21 (Adrenomedullin 1-52-Gly
(ADM 1-52-Gly): amino acids 95-147 of preproADM)
YRQSMN NFQGLRSFGC RFGTCTVQKL AHQIYQFTDK DKDNVAPRSK
ISPQGYG
SEQ ID No.: 22 (Murine ADM 1-50)
YRQSMNQGSRSNGCRFGTCTFQKLAHQIYQLTDKDKDGMAPRNKISPQG
Y-CONH₂
SEQ ID No.: 23 (1-42 of human ADM):
YRQSMNNFQGLRSFGCRFGTCTVQKLAHQIYQFTDKDKDNVA
SEQ ID No.: 24 (aa 43-52 of human ADM)
PRSKISPQGY-NH₂
SEQ ID No.: 25 (aa 1-14 of human ADM)
YRQSMNNFQGLRSF
SEQ ID No.: 26 (aa 1-10 of human ADM)
YRQSMNNFQG
SEQ ID No.: 27 (aa 1-6 of human ADM)
YRQSMN
SEQ ID No.: 28 (aa 1-32 of human ADM)
YRQSMNNFQGLRSFGCRFGTCTVQKLAHQIYQ
SEQ ID No.: 29 (aa 1-40 murine ADM)
YRQSMNQGSRSNGCRFGTCTFQKLAHQIYQLTDKDKDGMA
SEQ ID No.: 30 (aa 1-31 murine ADM)
YRQSMNQGSRSNGCRFGTCTFQKLAHQIYQL
SEQ ID No.: 31 (proADM: 164 amino acids
(22-185 of preproADM))
ARLDVASEF RKKWNKWALS RGKRELRMSS SYPTGLADVK
AGPAQTLIRP QDMKGASRSP EDSSPDAARI RVKRYRQSMN
NFQGLRSFGC RFGTCTVQKL AHQIYQFTDK DKDNVAPRSK
ISPQGYGRRR RRSLPEAGPG RTLVSSKPQA HGAPAPPSGS APHFL
SEQ ID No.: 32 (Proadrenomedullin N-20 terminal
peptide, PAMP: amino acids 22-41 of preproADM)
ARLDVASEF RKKWNKWALS R
SEQ ID No.: 33 (Midregional proAdrenomedullin,
MR-proADM: amino acids 45-92 of preproADM)
ELRMSS SYPTGLADVK AGPAQTLIRP QDMKGASRSP
EDSSPDAARI RV
SEQ ID No.: 34 (C-terminal proAdrenomedullin,
CT-proADM: amino acids 148-185 of preproADM)
RRR RRSLPEAGPG RTLVSSKPQA HGAPAPPSGS APHFL
SEQ ID No.: 35 (heavy chain, HAM8101)
QVQLVQSGAEVKKPGSSVKVSCKASGYTFSRYWIEWVRQAPGQGLEWIGE
ILPGSGSTNYNQKFQGRVTITADTSTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFScSVMHEALHNHYTQKSLSLSPGK
SEQ ID No.: 36 (light chain, HAM 8101)
DVVLTQSPLSLPVTLGQPASISCRSSQSIVYSNGNTYLEWYLQRPGQSPR
LLIYRVSNRFSGVPDRFSGSGSGTDFTLKISRVEAEDVGVYYCFQGSHIP
YTFGGGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
SEQ ID No.: 37 - IGHV1-69*11
QVQLVQSGAEVKKPGSSVKVSCKASGGTFSSYAISWVRQAPGQGLEWMGR
IIPILGTANYAQKFQGRVTITADESTSTAYMELSSLRSEDTAVYYCARYY
YYYGMDVWGQGTTVTVSS
SEQ ID No. 38: - HB3
QVQLQQSGAELMKPGASVKISCKATGYTFSRYWIEWVKQRPGHGLEWIGE
ILPGSGSTNYNEKFKGKATITADTSSNTAYMQLSSLTSEDSAVYYCTEGY
EYDGFDYWGQGTTLTVSS

1. A method for (a) diagnosing or predicting the risk of life-threatening deterioration or an adverse event or (b) diagnosing or prognosing the severity or (c) predicting or monitoring the success of a therapy or intervention or (d) therapy guidance or therapy stratification or (e) patient management in a patient infected with a Corona virus, the method comprising:
determining the level of pro-Adrenomedullin (SEQ ID No. 31) or fragment thereof in a sample of bodily fluid of said patient,
comparing said level of pro-Adrenomedullin or fragment thereof to a pre-determined threshold or to a previously measured level of pro-Adrenomedullin or fragment thereof, and
correlating said level of pro-Adrenomedullin or fragment thereof with the risk of life-threatening deterioration or an adverse event, or
correlating said level of pro-Adrenomedullin or fragment thereof with the severity, or
correlating said level of pro-Adrenomedullin or fragment thereof with the success of a therapy or intervention, or
correlating said level of pro-Adrenomedullin or fragment thereof with a certain therapy or intervention, or
correlating said level of pro-Adrenomedullin or fragment thereof with the management of said patient,
wherein said pro-Adrenomedullin or fragment thereof is selected from the group consisting of PAMP (SEQ ID No. 32), MR-proADM (SEQ ID No. 33), ADM-NH₂ (SEQ ID No. 20), ADM-Gly (SEQ ID No. 21) and CT-proADM (SEQ ID No. 34).

2. A method for (a) diagnosing or predicting the risk of life-threatening deterioration or an adverse event or (b) prognosing the severity or (c) predicting or monitoring the success of a therapy or intervention in a patient infected with a Corona virus according to claim 1, wherein said Corona Virus is selected from the group comprising Sars-CoV-1, Sars-CoV-2, MERS-CoV, in particular Sars-CoV-2.

3. A method for (a) diagnosing or predicting the risk of life-threatening deterioration or an adverse event or (b) prognosing the severity or (c) predicting or monitoring the success of a therapy or intervention in a patient infected with a Corona virus according to claim 1, wherein said adverse event is selected from the group comprising death, organ dysfunction, shock.

4. A method for (a) diagnosing or predicting the risk of life-threatening deterioration or an adverse event or (b) prognosing the severity or (c) predicting or monitoring the success of a therapy or intervention in a patient infected with a Corona virus according to claim 1, wherein said level of pro-Adrenomedullin or fragment thereof is above a pre-determined threshold.

5. A method for (a) diagnosing or predicting the risk of life-threatening deterioration or an adverse event or (b) prognosing the severity or (c) predicting or monitoring the success of a therapy or intervention in a patient infected with a Corona virus according to claim 1, wherein said patient has a level of D-dimer equal or greater than 0.5 μg/ml, preferably equal or greater than 1.0 μg/ml.

6. A method for (a) diagnosing or predicting the risk of life-threatening deterioration or an adverse event or (b) prognosing the severity or (c) predicting or monitoring the success of a therapy or intervention in a patient infected with a Corona virus according to claim 1, wherein the level of pro-Adrenomedullin or fragment thereof is determined by contacting said sample of bodily fluid with a capture binder that binds specifically to pro-Adrenomedullin or fragment thereof.

7. A method for (a) diagnosing or predicting the risk of life-threatening deterioration or an adverse event or (b) prognosing the severity or (c) predicting or monitoring the success of a therapy or intervention in a patient infected with a Corona virus according to claim 1, wherein said determination comprises the use of a capture-binder that binds specifically to pro-Adrenomedullin or fragment thereof wherein said capture-binder may be selected from the group of antibody, antibody fragment or non-IgG scaffold.

8. A method for (a) diagnosing or predicting the risk of life-threatening deterioration or an adverse event or (b) prognosing the severity or (c) predicting or monitoring the success of a therapy or intervention in a patient infected with a Corona virus according to claim 1, wherein said patient is treated with an Anti-adrenomedullin (ADM) antibody or anti-ADM antibody fragment or anti-ADM non-Ig scaffold wherein said anti-ADM antibody or anti-ADM fragment or anti-ADM non-Ig scaffold binds to the N-terminal and/or mid-regional part (aa 1-42) of ADM-Gly and/or ADM-NH₂:
(SEQ ID No. 23)
YRQSMNNFQGLRSFGCRFGTCTVQKLAHQIYQFTDKDKDNVA.

9. Anti-adrenomedullin (ADM) antibody or anti-ADM antibody fragment or anti-ADM non-Ig scaffold for use in therapy or intervention in a patient infected with a Corona virus.

10. Anti-adrenomedullin (ADM) antibody or anti-ADM antibody fragment or anti-ADM non-Ig scaffold for use in therapy or intervention in a patient infected with a Corona virus according to claim 9, wherein said Corona Virus is selected from the group comprising Sars-CoV-1, Sars-CoV-2, MERS-CoV, in particular Sars-CoV-2.

11. Anti-adrenomedullin (ADM) antibody or anti-ADM antibody fragment or anti-ADM non-Ig scaffold for use in therapy or intervention in a patient infected with a Corona virus, wherein said patient has a level of pro-Adrenomedullin or fragment thereof in a sample of bodily fluid of said subject that is above a predetermined threshold or higher than a previously measured level of pro-Adrenomedullin when determined by a method according to claim 1.

12. Anti-adrenomedullin (ADM) antibody or anti-ADM antibody fragment or anti-ADM non-Ig scaffold for use in therapy or intervention in a patient infected with a Corona virus according to claim 9, wherein said patient has a level of D-dimer equal or greater than 0.5 μg/ml, preferably equal or greater than 1.0 μg/ml.

13. Anti-adrenomedullin (ADM) antibody or anti-ADM antibody fragment or anti-ADM non-Ig scaffold for use in therapy or intervention in a patient infected with a Corona virus according to claim 9, wherein said anti-ADM antibody or anti-ADM fragment or anti-ADM non-Ig scaffold binds to the N-terminal (amino acid 1-21) of ADM-Gly and/or ADM-NH₂: YRQSMNNFQGLRSFGCRFGTC (SEQ ID No. 14).

14. Anti-adrenomedullin (ADM) antibody or anti-ADM antibody fragment or anti-ADM non-Ig scaffold for use in therapy or intervention in a patient infected with a Corona virus according to claim 9, wherein said antibody is a monoclonal antibody or monoclonal antibody fragment.

15. Anti-adrenomedullin (ADM) antibody or anti-ADM antibody fragment or anti-ADM non-Ig scaffold for use in therapy or intervention in a patient infected with a Corona virus according to claim 14, wherein the complementarity determining regions (CDR's) in the heavy chain comprises the sequences:
CDR1:
SEQ ID NO: 1
GYTFSRYW
CDR2:
SEQ ID NO: 2
ILPGSGST
CDR3:
SEQ ID NO: 3
TEGYEYDGFDY
and the complementarity determining regions (CDR's) in the light chain comprises the sequences:
CDR1:
SEQ ID NO: 4
QSIVYSNGNTY
CDR2:
RVS
CDR3:
SEQ ID NO: 5
FQGSHIPYT

16. Anti-adrenomedullin (ADM) antibody or anti-ADM antibody fragment or anti-ADM non-Ig scaffold for use in therapy or intervention in a patient infected with a Corona virus according to claim 15, wherein said antibody or fragment comprises a sequence selected from the group comprising as a VH region:
(AM-VH-C)
SEQ ID NO: 6
QVQLQQSGAELMKPGASVKISCKATGYTFSRYWIEWVKQRPGHGLEWIGE
ILPGSGSTNYNEKFKGKATITADTSSNTAYMQLSSLTSEDSAVYYCTEGY
EYDGFDYWGQGTTLTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPK
(AM-VH1)
SEQ ID NO: 7
QVQLVQSGAEVKKPGSSVKVSCKASGYTFSRYWISWVRQAPGQGLEWMGR
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPK
(AM-VH2-E40)
SEQ ID NO: 8
QVQLVQSGAEVKKPGSSVKVSCKASGYTFSRYWIEWVRQAPGQGLEWMGR
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPK
(AM-VH3-T26-E55)
SEQ ID NO: 9
QVQLVQSGAEVKKPGSSVKVSCKATGYTFSRYWISWVRQAPGQGLEWMGE
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPK
(AM-VH4-T26-E40-E55)
SEQ ID NO: 10
QVQLVQSGAEVKKPGSSVKVSCKATGYTFSRYWIEWVRQAPGQGLEWMGE
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPK
or a sequence that is >80% identical to each of the above depicted sequences respectively, and
comprises a sequence selected from the group comprising the following sequence as a VL region:
(AM-VL-C)
SEQ ID NO: 11
DVLLSQTPLSLPVSLGDQATISCRSSQSIVYSNGNTYLEWYLQKPGQSPK
LLIYRVSNRFSGVPDRFSGSGSGTDFTLKISRVEAEDLGVYYCFQGSHIP
YTFGGGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
(AM-VL1)
SEQ ID NO: 12
DVVMTQSPLSLPVTLGQPASISCRSSQSIVYSNGNTYLNWFQQRPGQSPR
RLIYRVSNRDSGVPDRFSGSGSGTDFTLKISRVEAEDVGVYYCFQGSHIP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
(AM-VL2-E40)
SEQ ID NO: 13
DVVMTQSPLSLPVTLGQPASISCRSSQSIVYSNGNTYLEWFQQRPGQSPR
RLIYRVSNRDSGVPDRFSGSGSGTDFTLKISRVEAEDVGVYYCFQGSHIP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
or a sequence that is >80% identical to each of the above depicted sequences.

17. Adrenomedullin (ADM) antibody or anti-ADM antibody fragment or anti-ADM non-Ig scaffold for use in therapy or intervention in a patient infected with a Corona virus according to claim 15, wherein said antibody or fragment comprises the following sequence as a heavy chain:
SEQ ID NO: 35
QVQLVQSGAEVKKPGSSVKVSCKASGYTFSRYWIEWVRQAPGQGLEWIGE
ILPGSGSTNYNQKFQGRVTITADTSTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK
or a sequence that is >95% identical to it,
and comprises the following sequence as a light chain:
SEQ ID NO: 36
DVVLTQSPLSLPVTLGQPASISCRSSQSIVYSNGNTYLEWYLQRPGQSPR
LLIYRVSNRFSGVPDRFSGSGSGTDFTLKISRVEAEDVGVYYCFQGSHIP
YTFGGGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
or a sequence that is >95% identical to it.

18. Anti-adrenomedullin (ADM) antibody or anti-ADM antibody fragment or anti-ADM non-Ig scaffold for use in therapy or intervention in a patient with compromised lung function and/or acute respiratory distress syndrome (ARDS).

19. Anti-adrenomedullin (ADM) antibody or anti-ADM antibody fragment or anti-ADM non-Ig scaffold for use in therapy or intervention in a patient with compromised lung function and/or acute respiratory distress syndrome (ARDS) according to claim 18, wherein said patient has a Horowitz index below 300, in particular below 200, in particular below 100 and/or said patient is in need of mechanical ventilation.

20. Anti-adrenomedullin (ADM) antibody or anti-ADM antibody fragment or anti-ADM non-Ig scaffold for use in therapy or intervention in a patient with compromised lung function and/or acute respiratory distress syndrome (ARDS), wherein said patient has a level of pro-Adrenomedullin or fragment thereof in a sample of bodily fluid of said subject that is above a predetermined threshold or higher than a previously measured level of pro-Adrenomedullin when determined by a method according to claim 1.

21. Anti-adrenomedullin (ADM) antibody or anti-ADM antibody fragment or anti-ADM non-Ig scaffold for use in therapy or intervention in a patient with compromised lung function and/or acute respiratory distress syndrome (ARDS) according to claim 18, wherein the complementarity determining regions (CDR's) in the heavy chain comprises the sequences:
CDR1:
SEQ ID NO: 1
GYTFSRYW
CDR2:
SEQ ID NO: 2
ILPGSGST
CDR3:
SEQ ID NO: 3
TEGYEYDGFDY
and the complementarity determining regions (CDR's) in the light chain comprises the sequences:
CDR1:
SEQ ID NO: 4
QSIVYSNGNTY
CDR2:
RVS
CDR3:
SEQ ID NO: 5
FQGSHIPYT

22. Anti-adrenomedullin (ADM) antibody or anti-ADM antibody fragment or anti-ADM non-Ig scaffold for use in therapy or intervention in a patient with compromised lung function and/or acute respiratory distress syndrome (ARDS) according to claim 21, wherein said antibody or fragment comprises a sequence selected from the group comprising as a VH region:
(AM-VH-C)
SEQ ID NO: 6
QVQLQQSGAELMKPGASVKISCKATGYTFSRYWIEWVKQRPGHGLEWIGE
ILPGSGSTNYNEKFKGKATITADTSSNTAYMQLSSLTSEDSAVYYCTEGY
EYDGFDYWGQGTTLTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPK
(AM-VH1)
SEQ ID NO: 7
QVQLVQSGAEVKKPGSSVKVSCKASGYTFSRYWISWVRQAPGQGLEWMGR
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPK
(AM-VH2-E40)
SEQ ID NO: 8
QVQLVQSGAEVKKPGSSVKVSCKASGYTFSRYWIEWVRQAPGQGLEWMGR
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPK
(AM-VH3-T26-E55)
SEQ ID NO: 9
QVQLVQSGAEVKKPGSSVKVSCKATGYTFSRYWISWVRQAPGQGLEWMGE
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPK
(AM-VH4-T26-E40-E55)
SEQ ID NO: 10
QVQLVQSGAEVKKPGSSVKVSCKATGYTFSRYWIEWVRQAPGQGLEWMGE
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPK
or a sequence that is >80% identical to each of the above depicted sequences respectively, and
comprises a sequence selected from the group comprising the following sequence as a VL region:
(AM-VL-C)
SEQ ID NO: 11
DVLLSQTPLSLPVSLGDQATISCRSSQSIVYSNGNTYLEWYLQKPGQSPK
LLIYRVSNRFSGVPDRFSGSGSGTDFTLKISRVEAEDLGVYYCFQGSHIP
YTFGGGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
(AM-VL1)
SEQ ID NO: 12
DVVMTQSPLSLPVTLGQPASISCRSSQSIVYSNGNTYLNWFQQRPGQSPR
RLIYRVSNRDSGVPDRFSGSGSGTDFTLKISRVEAEDVGVYYCFQGSHIP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
(AM-VL2-E40)
SEQ ID NO: 13
DVVMTQSPLSLPVTLGQPASISCRSSQSIVYSNGNTYLEWFQQRPGQSPR
RLIYRVSNRDSGVPDRFSGSGSGTDFTLKISRVEAEDVGVYYCFQGSHIP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
or a sequence that is >80% identical to each of the above depicted sequences.

23. Adrenomedullin (ADM) antibody or anti-ADM antibody fragment or anti-ADM non-Ig scaffold for use in therapy or intervention in a patient with compromised lung function and/or acute respiratory distress syndrome (ARDS) according to claim 21, wherein said antibody or fragment comprises the following sequence as a heavy chain:
SEQ ID NO: 35
QVQLVQSGAEVKKPGSSVKVSCKASGYTFSRYWIEWVRQAPGQGLEWIGE
ILPGSGSTNYNQKFQGRVTITADTSTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK
or a sequence that is >95% identical to it,
and comprises the following sequence as a light chain:
SEQ ID NO: 36
DVVLTQSPLSLPVTLGQPASISCRSSQSIVYSNGNTYLEWYLQRPGQSPR
LLIYRVSNRFSGVPDRFSGSGSGTDFTLKISRVEAEDVGVYYCFQGSHIP
YTFGGGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
or a sequence that is >95% identical to it.


SEQUENCE LISTING (38 sequences)

SEQ ID NO: 1 - protein, 8 aa, Homo sapiens
GYTFSRYW

SEQ ID NO: 2 - protein, 8 aa, Homo sapiens
ILPGSGST

SEQ ID NO: 3 - protein, 11 aa, Homo sapiens
TEGYEYDGFDY

SEQ ID NO: 4 - protein, 11 aa, Homo sapiens
QSIVYSNGNTY

SEQ ID NO: 5 - protein, 9 aa, Homo sapiens
FQGSHIPYT

SEQ ID NO: 6 - protein, 219 aa, Homo sapiens
QVQLQQSGAELMKPGASVKISCKATGYTFSRYWIEWVKQRPGHGLEWIGEILPGSGSTNYNEKFKGKATITADTSSNTAYMQLSSLTSEDSAVYYCTEGYEYDGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPK

SEQ ID NO: 7 - protein, 219 aa, Homo sapiens
QVQLVQSGAEVKKPGSSVKVSCKASGYTFSRYWISWVRQAPGQGLEWMGRILPGSGSTNYAQKFQGRVTITADESTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPK

SEQ ID NO: 8 - protein, 219 aa, Homo sapiens
QVQLVQSGAEVKKPGSSVKVSCKASGYTFSRYWIEWVRQAPGQGLEWMGRILPGSGSTNYAQKFQGRVTITADESTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPK

SEQ ID NO: 9 - protein, 219 aa, Homo sapiens
QVQLVQSGAEVKKPGSSVKVSCKATGYTFSRYWISWVRQAPGQGLEWMGEILPGSGSTNYAQKFQGRVTITADESTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPK

SEQ ID NO: 10 - protein, 219 aa, Homo sapiens
QVQLVQSGAEVKKPGSSVKVSCKATGYTFSRYWIEWVRQAPGQGLEWMGEILPGSGSTNYAQKFQGRVTITADESTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPK

SEQ ID NO: 11 - protein, 219 aa, Homo sapiens
DVLLSQTPLSLPVSLGDQATISCRSSQSIVYSNGNTYLEWYLQKPGQSPKLLIYRVSNRFSGVPDRFSGSGSGTDFTLKISRVEAEDLGVYYCFQGSHIPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 219 aa, Homo sapiens
DVVMTQSPLSLPVTLGQPASISCRSSQSIVYSNGNTYLNWFQQRPGQSPRRLIYRVSNRDSGVPDRFSGSGSGTDFTLKISRVEAEDVGVYYCFQGSHIPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 219 aa, Homo sapiens
DVVMTQSPLSLPVTLGQPASISCRSSQSIVYSNGNTYLEWFQQRPGQSPRRLIYRVSNRDSGVPDRFSGSGSGTDFTLKISRVEAEDVGVYYCFQGSHIPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 21 aa, Homo sapiens
YRQSMNNFQGLRSFGCRFGTC

SEQ ID NO: 15 - protein, 12 aa, Homo sapiens
CTVQKLAHQIYQ

SEQ ID NO: 16 - protein, 12 aa, Homo sapiens
CAPRSKISPQGY

SEQ ID NO: 17 - protein, 19 aa, Mus musculus
YRQSMNQGSRSNGCRFGTC

SEQ ID NO: 18 - protein, 12 aa, Mus musculus
CTFQKLAHQIYQ

SEQ ID NO: 19 - protein, 12 aa, Mus musculus
CAPRNKISPQGY

SEQ ID NO: 20 - protein, 52 aa, Homo sapiens
YRQSMNNFQGLRSFGCRFGTCTVQKLAHQIYQFTDKDKDNVAPRSKISPQGY

SEQ ID NO: 21 - protein, 53 aa, Homo sapiens
YRQSMNNFQGLRSFGCRFGTCTVQKLAHQIYQFTDKDKDNVAPRSKISPQGYG

SEQ ID NO: 22 - protein, 50 aa, Mus musculus
YRQSMNQGSRSNGCRFGTCTFQKLAHQIYQLTDKDKDGMAPRNKISPQGY

SEQ ID NO: 23 - protein, 42 aa, Homo sapiens
YRQSMNNFQGLRSFGCRFGTCTVQKLAHQIYQFTDKDKDNVA

SEQ ID NO: 24 - protein, 10 aa, Homo sapiens
PRSKISPQGY

SEQ ID NO: 25 - protein, 14 aa, Homo sapiens
YRQSMNNFQGLRSF

SEQ ID NO: 26 - protein, 10 aa, Homo sapiens
YRQSMNNFQG

SEQ ID NO: 27 - protein, 6 aa, Homo sapiens
YRQSMN

SEQ ID NO: 28 - protein, 32 aa, Homo sapiens
YRQSMNNFQGLRSFGCRFGTCTVQKLAHQIYQ

SEQ ID NO: 29 - protein, 40 aa, Mus musculus
YRQSMNQGSRSNGCRFGTCTFQKLAHQIYQLTDKDKDGMA

SEQ ID NO: 30 - protein, 31 aa, Mus musculus
YRQSMNQGSRSNGCRFGTCTFQKLAHQIYQL

SEQ ID NO: 31 - protein, 164 aa, Homo sapiens
ARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 32 - protein, 20 aa, Homo sapiens
ARLDVASEFRKKWNKWALSR

SEQ ID NO: 33 - protein, 48 aa, Homo sapiens
ELRMSSSYPTGLADVKAGPAQTLIRPQDMKGASRSPEDSSPDAARIRV

SEQ ID NO: 34 - protein, 38 aa, Homo sapiens
RRRRRSLPEAGPGRTLVSSKPQAHGAPAPPSGSAPHFL

SEQ ID NO: 35 - protein, 448 aa, Homo sapiens
QVQLVQSGAEVKKPGSSVKVSCKASGYTFSRYWIEWVRQAPGQGLEWIGEILPGSGSTNYNQKFQGRVTITADTSTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 219 aa, Homo sapiens
DVVLTQSPLSLPVTLGQPASISCRSSQSIVYSNGNTYLEWYLQRPGQSPRLLIYRVSNRFSGVPDRFSGSGSGTDFTLKISRVEAEDVGVYYCFQGSHIPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 118 aa, Homo sapiens
QVQLVQSGAEVKKPGSSVKVSCKASGGTFSSYAISWVRQAPGQGLEWMGRIIPILGTANYAQKFQGRVTITADESTSTAYMELSSLRSEDTAVYYCARYYYYYGMDVWGQGTTVTVSS

SEQ ID NO: 38 - protein, 118 aa, Homo sapiens
QVQLQQSGAELMKPGASVKISCKATGYTFSRYWIEWVKQRPGHGLEWIGEILPGSGSTNYNEKFKGKATITADTSSNTAYMQLSSLTSEDSAVYYCTEGYEYDGFDYWGQGTTLTVSS